(19) United States
(12) Patent Application Publication
(10) Pub. No.: US 2005/0245432 A1
(43) Pub. Date: Nov. 3, 2005

(54) N-methyl amino acids

(76) Inventors: Luigi Aurelio, Victoria (AU); John S. Box, Victoria (AU); Robert T.C. Brownlee, Victoria (AU); Andrew B. Hughes, Victoria (AU)

Correspondence Address: NEEDLE & ROSENBERG, P.C., SUITE 1000, 999 PEACHTREE STREET, ATLANTA, GA, 30309-3915, US

(21) Appl. No.: 10/520,843

(22) PCT Filed: Jul. 11, 2003
(86) PCT No.: PCT/AU03/00908

§ 371 Date: Jun. 24, 2005

(30) Foreign Application Priority Data
Jul. 11, 2002 (AU) 2002950136

(51) Int. Cl. A61K 38/17; A61K 31/4439; A61K 31/421; A61K 31/4172; A61K 31/198

(52) U.S. Cl. 514/2; 514/357; 514/340; 514/400; 514/562; 514/563; 530/409; 546/271.4; 546/335; 548/335.5; 548/225; 548/495; 562/450; 562/557

(57) ABSTRACT

The present invention relates to a compound of formula (I) or (II), processes for preparing them, peptides including them and kits involving them.

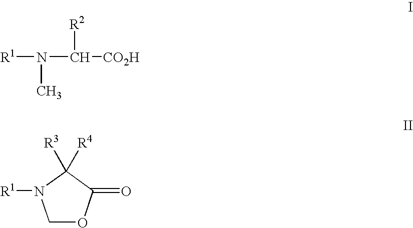

The present invention relates to new N-methyl amino acids and their precursor oxazolidinones, processes for their preparation and their use in the synthesis of peptides. The invention also includes the use of the new N-methyl amino acids together with known N-methyl amino acids in a kit for synthesising peptides.

### BACKGROUND OF THE INVENTION

N-methyl amino acids are secondary metabolites present in a wide variety of naturally occurring peptides that display a remarkable range of biological activities including antibiotic, antiviral, anticancer and antifungal. They are also useful compounds for increasing certain pharmacokinetic parameters such as membrane permeability, proteolytic stability and conformational rigidity. In view of the limited availability of N-methyl amino acids, there is a need to prepare such compounds and their precursors for use in the solution and solid phase synthesis of target peptides.

A range of methods have been employed to prepare N-methyl amino acids. These include methods for direct methylation, ¹⁻⁵ reductive amination, ⁶⁻¹² alternative methods ¹³⁻¹⁸ and through the generation of oxazolidinones and their subsequent transformation to the N-methyl product. ¹⁹⁻²³ In addition, there are strategies involving the use of immonium ions in Diels-Alder/retro-Diels-Alder sequences,²⁴ the nucleophilic displacement of triflates,²⁵ the hydroxyamination of chiral enolates²⁶ and the Mitsunobu reaction.²⁷ Some of these methods suffer from limitations in the range of amino acids to which they are applicable, some utilize rather long synthetic sequences and some cause at least partial racemisation of the substrate. We have exploited oxazolidinone chemistry to generate a range of N-methyl amino acids and their precursor oxazolidinones.

The general oxazolidinone route is shown below, where protected amino acids are cyclized efficiently to oxazolidinones. These oxazolidinones may be reductively cleaved by complementary procedures that give N-methyl amino acids.

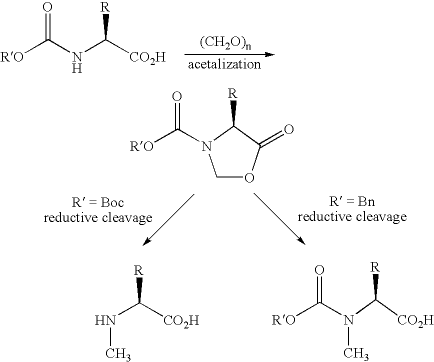

We previously reported²⁷ the synthesis, via the carbamates (1) and the 5-oxazolidinones (2), of a number of new N-methyl α-amino acids mainly in the form of their benzyl carbamates (3) and free amino acids (4) as shown below.

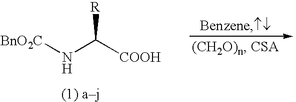

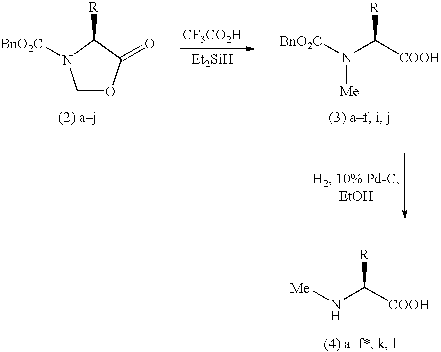

| | |
|---|---|
| a | R = CH₂OH |
| b | R = CH(OH)CH₃ |
| c | R = CH₂PhOH |
| d | R = CH₂CH₂COOH (DL) |
| e | R = Ph |
| f | R = CH₂CH₂CH₂CH₂NPhth |
| g | R = CH₂SH |
| h | R = CH₂CH₂SCH₃ |
| i | R = CH₂COOBn |
| j | R = CH₂CH₂CONHCbz |
| k | R = CH₂CH₂CONH₂ |
| l | R = CH₂COOH |

*(4f) isolated as p-TsOH salt

A focus of that study was the endeavour to demonstrate the general applicability of 5-oxazolidinones to the generation of N-methyl derivatives of the 20 common natural α-amino acids in the absence of (eg glutamic acid and tyrosine) or the minimal presence (eg glutamine and aspartic acid) of side chain protecting groups. This approach was designed to emphasise the efficiency of the oxazolidinone route, its mildness, as measured by the lack of racemisation of the α-center, and its chemoselectivity. Indeed, the selectivity of the oxazolidination reaction for the α-amino acid backbone aza and carboxylic functionalities often allowed the subsequent manipulation of reactive sidechains.

However in that paper²⁷ there were notable failures in the strategy particularly in regard to certain difficult α-amino acids, those bearing sidechains such as histidine and tryptophan. We have now successfully synthesised these outstanding N-methyl targets, together with others such as threonine, serine, cysteine, methionine, asparagine, aspartic acid and glutamic acid and their oxazolidinone precursors. As a result, the 5-oxazolidinone route to N-methyl amino acids has now been applied to the synthesis of all 20 of the common L-α-amino acids and some related compounds.

### SUMMARY OF THE INVENTION

According to the present invention there is provided a compound of formula I or II: 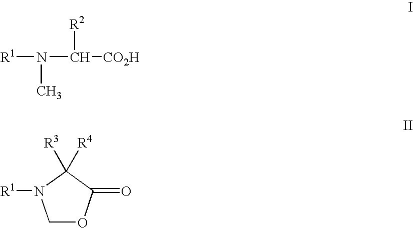
in which
R¹ is an N-protecting group or a peptide;
R² is CHCH₃OAc or CHR⁵R⁶ in which R⁵ is hydrogen and R⁶ is OAc, CONH₂, SBn, 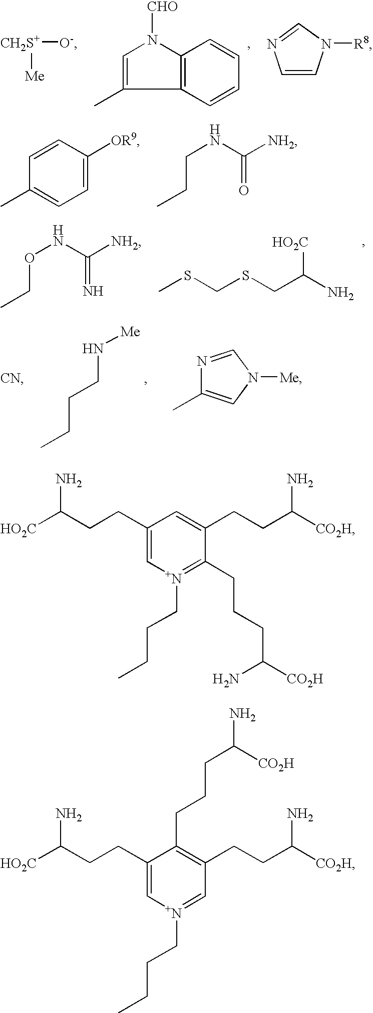
   CO₂R⁷ or CH₂CO₂R⁷ in which R⁷ is a carboxyl protecting group; and
R³ is CHCH₃OAc, 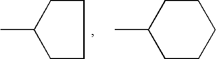
   or CHR⁵R⁶ in which R⁵ is as defined above and R⁶ is OAc, SBn, CONHTrt, 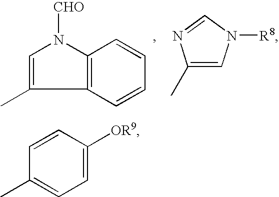
   CO₂R⁷, CHCO₂R⁷, CH₂CH₃ or CH═CH₂ in which R⁷ is as defined above, R⁸ is a histidine protecting group and R⁹ is a phenol protecting group;
R⁴ is hydrogen or R⁴ is methyl when R³ is OAc;
R³ together with R⁴ forms cyclopentyl; or
R² and R³ independently represent optionally protected amino acid side chains selected from: 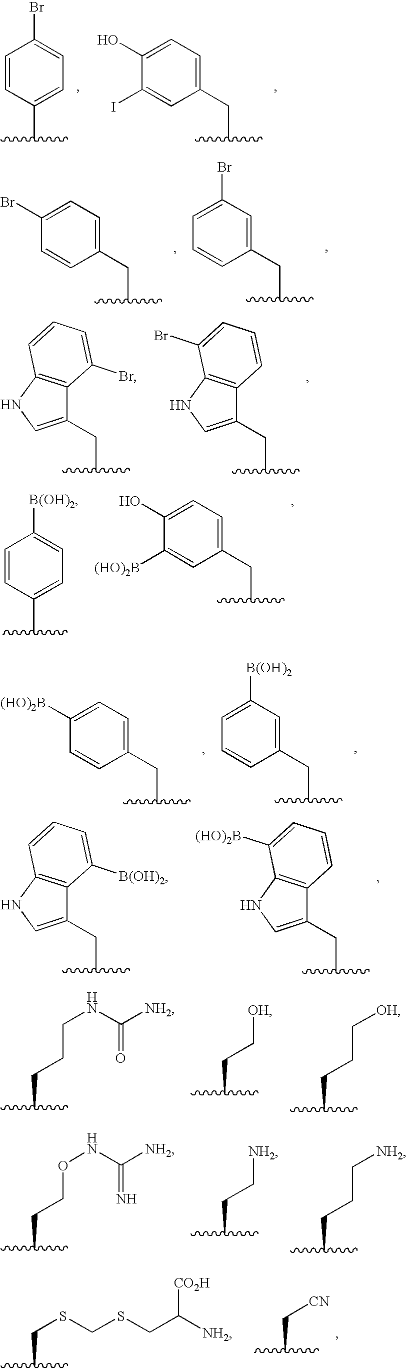 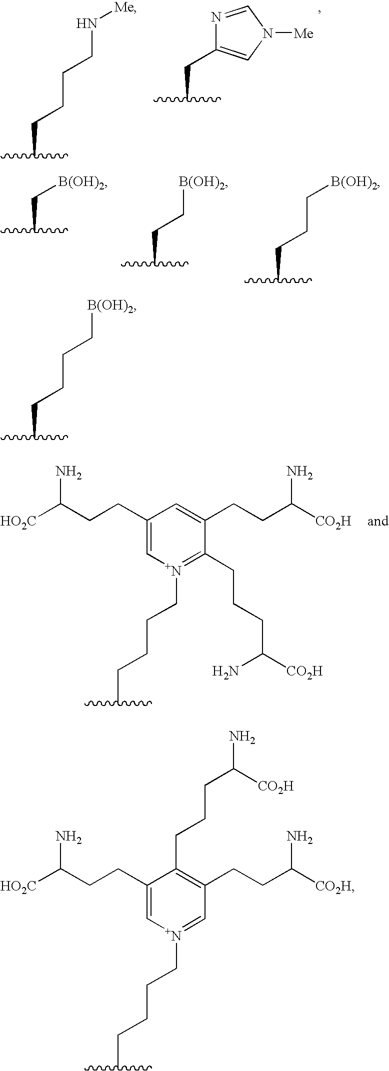
salts, hydrates, solvates, derivatives, tautomers and/or isomers thereof.

The present invention also provides a process for preparing the compound of formula I as defined above which comprises reductive cleavage of the compound of formula II defined above.

The present invention further provides a process for preparing the compound of formula I or II when
R¹ is an N-protecting group or a peptide;
R² is CHCH₃OAc or CHR⁵R⁶ in which R⁵ is hydrogen and R⁶ is OAc, CONH₂, SBn, 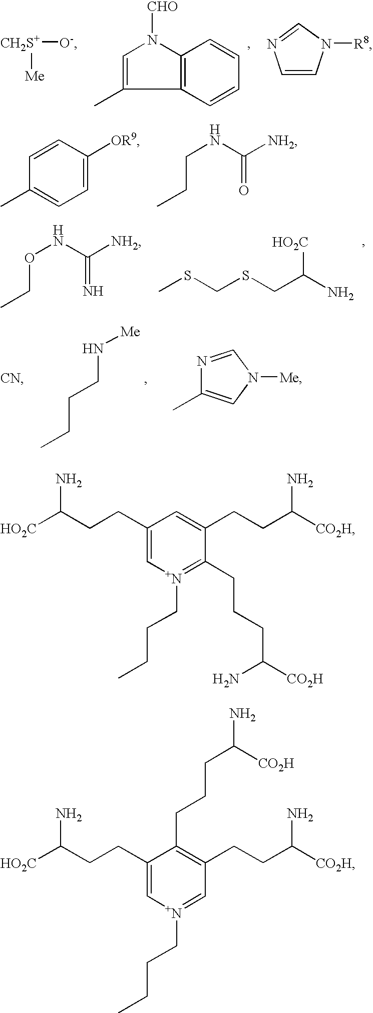
   CO₂R⁷ or CH₂CO₂R⁷ in which R⁷ is a carboxyl protecting group; and
R³ is CHCH₃OAc, 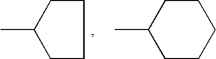
   or CHR⁵R⁶ in which R⁵ is as defined above and R⁶ is OAc, SBn, CONHTrt, 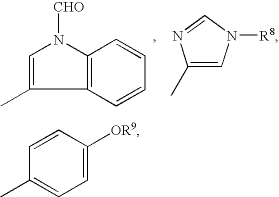
   CO₂R⁷, CHCO₂R⁷, CH₂CH₃ or CH═CH₂ in which R⁷ is as defined above, R⁸ is a histidine protecting group and R⁹ is a phenol protecting group; and
R⁴ is hydrogen or R⁴ is methyl when R³ is OAc; or
R³ together with R⁴ forms cyclopentyl, which comprises the steps of:
(a) converting a compound of formula III 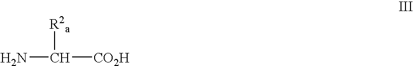
   in which
R²ₐ is CHOHMe or CHR⁵R⁶ₐ in which R⁵ is as defined above and R⁶ₐ is OH, SH, CONH₂, 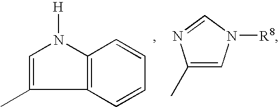
   in which R⁸ is as defined above, 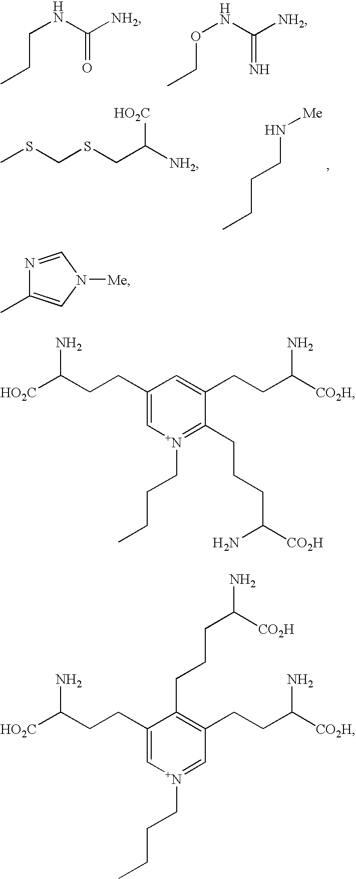
   CO₂H or CH₂CONH₂
or salts thereof
into a compound of formula IV 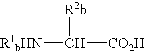
   in which
R¹_{b} is an N-protecting group;
R²_{b} is CHOAcMe or CHR⁵R⁶_{b} in which R⁵ is as defined above and R⁶_{b} is OAc, SBn, SMe, CONHR¹_{b} in which R¹_{b} is as defined above, 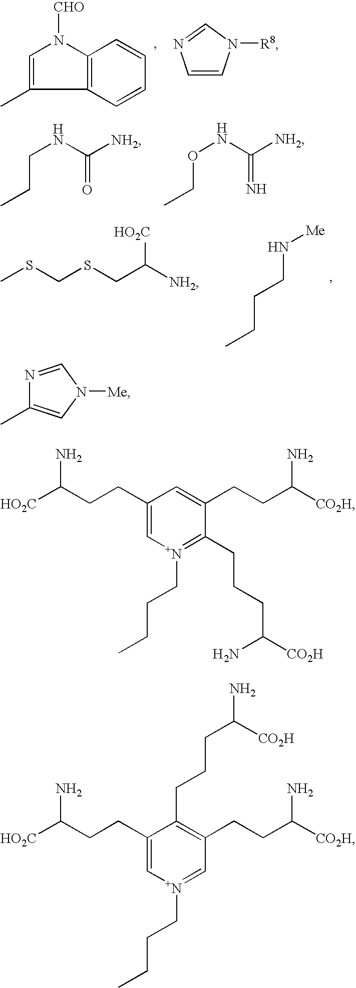
   CO₂H or CH₂CO₂H;
(b) oxazolidination of the compound of formula IV to form the compound of formula II as defined above; and
(c) reductive cleavage of the compound of formula II as defined above to form the compound of formula I as defined above.

Further according to the present invention there is provided use of the compound of formula I or II defined above in the synthesis of peptides.

Still further according to the present invention there is provided a peptide which includes a compound of formula I or II as defined above,

The invention also extends to a kit for use in synthesising peptides which comprises
(a) at least one compound of formula I or formula II or the peptide defined above; and
(b) optionally at least one other N-methyl amino acid, its precursor oxazolidinone, an optionally protected amino acid or protected forms thereof,
said compounds, N-methyl amino acids, oxazolidinones and/or amino acids being held separately.

### DETAILED DESCRIPTION OF THE INVENTION

For the purposes of this specification it will be clearly understood that the word “comprising” means “including but not limited to”, and that the word “comprises” has a corresponding meaning.

The term “N-protecting group” is used herein in its broadest sense and refers to any group capable of protecting the amino group of an amino acid such as those disclosed in Greene, T. W., “Protective Groups in Organic Synthesis” John Wiley & Sons, New York 1991, pp 315-398 and 379-385, the contents of which are incorporated herein by reference.

Preferably the N-protecting group is a carbamate such as, 9-fluorenylmethyl carbamate (Fmoc), 2,2,2-trichloroethyl carbamate (Troc), t-butyl carbamate (BOC), allyl carbamate (Alloc), 2-trimethylsilylethyl (Teoc) and benzyl carbamate (Cbz or Z), more preferably Fmoc or Z.

The term “carboxyl-protecting group” is used herein in its broadest sense and refers to any group capable of protecting a carboxyl group such as those disclosed in Green, T. W., “Protective Groups in Organic Synthesis” John Wiley & Sons, New York 1991, pp 224-276, the contents of which are incorporated herein by reference.

The term “histidine protecting group” is used herein in its broadest sense and refers to any group capable of protecting a histidine group such as carbamates, sulphonyl groups or N-aryl groups for example Z, tosyl, mesyl or 2,4-dinitrophenyl (DNP).

The term “phenol protecting group” is used herein in its broadest sense and refers to any group capable of protecting a phenol group in particular a tyrosine phenol group for example 2,4-DNP, acyl, alkyl or benzyl.

The term “amino acid side chain protecting group” is used herein in its broadest sense and refers to any suitable known group which is capable of protecting organic functionalities, for example, alcohols, amines, acids, amides or thiols, such as those disclosed in Greene, .W., “Protective Groups in Organic Synthesis” John Wiley & Sons, New York 1991. For example, the carboxyl groups of aspartic acid, glutamic acid and α-aminoadipic acid may be esterified (for example as a C₁-C₆ alkyl ester), the amino groups of lysine, ornithine and 5-hydroxylysine, may be converted to carbamates (for example as a C(═O)OC₁-C₆ alkyl or C(═O)OCH₂Ph carbamate) or imides such as phthalimide or succinimide, the hydroxyl groups of 5-hydroxylysine, 4-hydroxyproline, serine, threonine, tyrosine, 3,4-dihydroxyphenylalanine, homoserine, α-methylserine and thyroxine may be converted to ethers (for example a C₁-C₆ alkyl or a (C₁-C₆ alkyl)phenyl ether) or esters (for example a C═OC₁-C₆ alkyl ester) and the thiol group of cysteine may be converted to thioethers (for example a C₁-C₆ alkyl thioether) or thioesters (for example a C(═O)C₁-C₆ alkyl thioester).

The salts of the compound of Formula I, II or III are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

In addition, some of the compounds of the present invention may form solvates with water or common organic solvents. Such solvates are encompassed within the scope of the invention.

By “pharmaceutically acceptable derivative” is meant any pharmaceutically acceptable salt, hydrate, ester, amide, active metabolite, analogue, residue or any other compound which is not biologically or otherwise undesirable and induces the desired pharmacological and/or physiological effect.

The term “tautomer” is used herein in its broadest sense to include compounds of Formula I or II which are capable of existing in a state of equilibrium between two isomeric forms. Such compounds may differ in the bond connecting two atoms or groups and the position of these atoms or groups in the compound.

The term “isomer” is used herein in its broadest sense and includes structural, geometric and stereo isomers. As the compound of Formula I or II may have one or more chiral centres, it is capable of existing in enantiomeric forms and/or diastereomeric forms.

Representative examples of compounds of formula I are as follows: 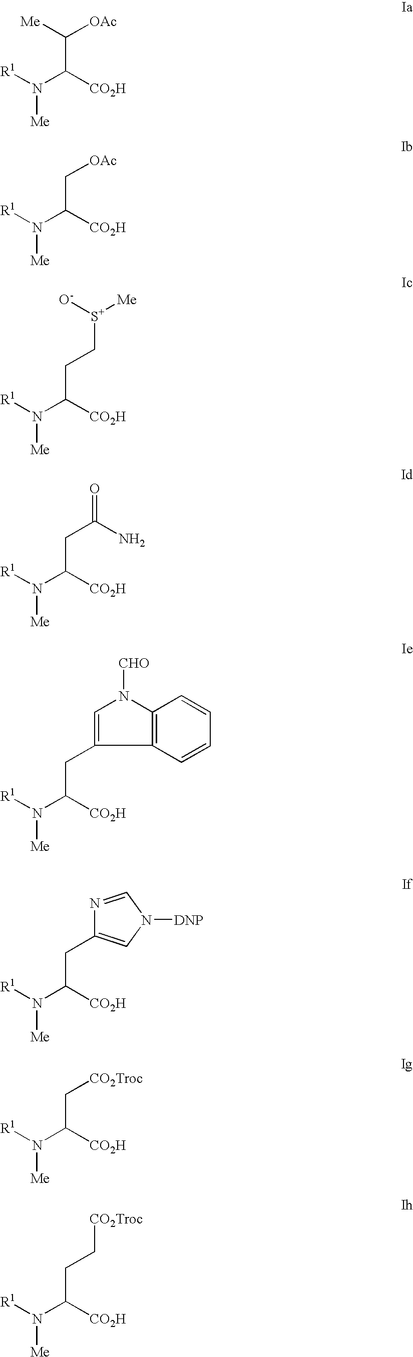 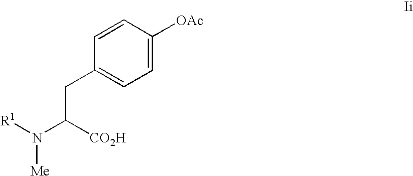
in which
R¹ is as defined above.

It will be appreciated that these compounds may be present as salts such as dicyclohexylammonium (DCHA) or tert-butylammonium salts.

Representative examples of compounds of formula II are as follows:

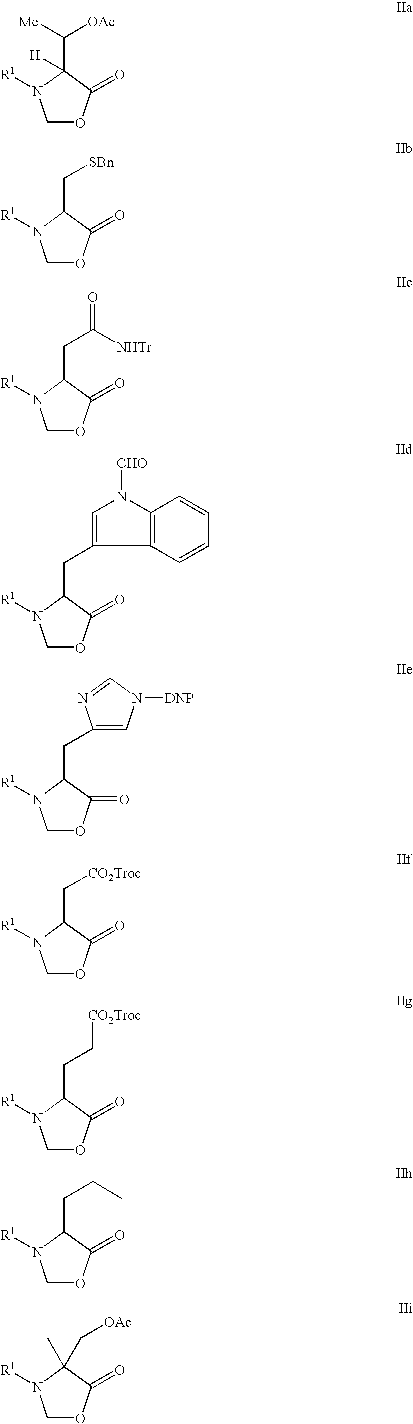

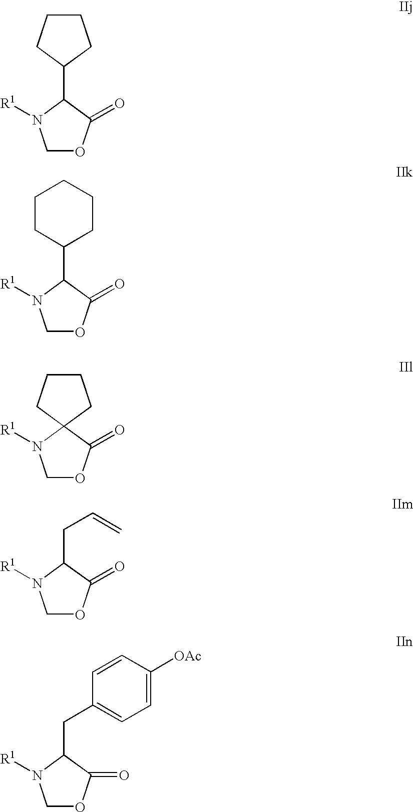

The reductive cleavage may be performed using any suitable known technique, preferably the method described by Freidinger et al²⁸ which employs trifluoroacetic acid (TFA) as the acid and triethylsilane (Et₃SiH) as the reductant.

Conversion step (a) results in the protection of the amino group on the compound of formula III to produce a compound of formula IV. This step may be performed using any suitable known technique, such as those disclosed in Greene, T. W., “Protective Groups in Organic Synthesis” John Wiley & Sons, New York, 1991.

Step (b) results in cyclisation of the compound of formula IV using any suitable known technique such as described by Aurelio, L. et al²⁷ using a formaldehyde source for example paraformaldehyde and paratoluenesulphonic acid (TsOH) in a suitable organic solvent such as benzene or toluene.

The preferred preparations of compounds (Ia) to (IIn) described above are shown in Schemes 1 to 9a below.

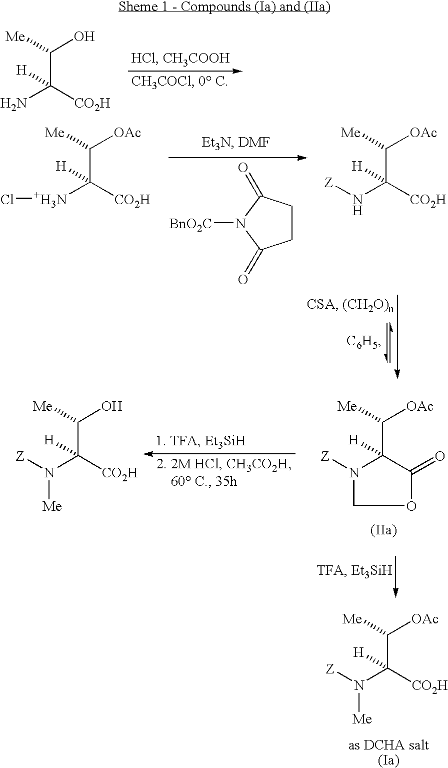

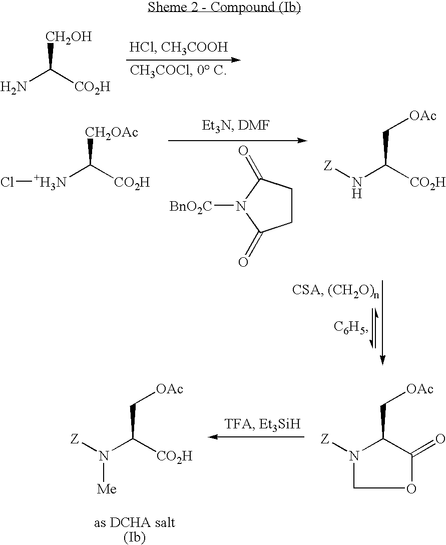

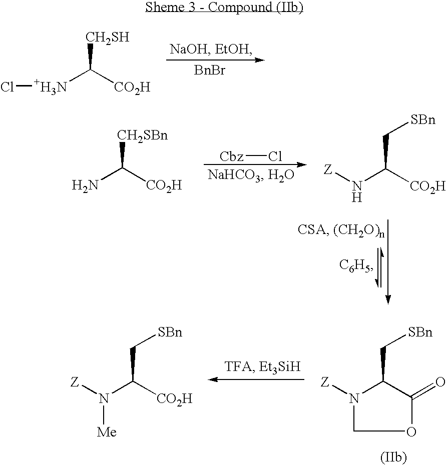

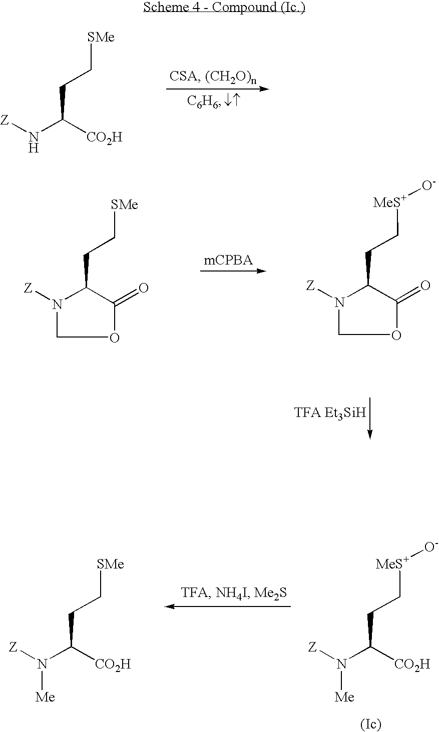

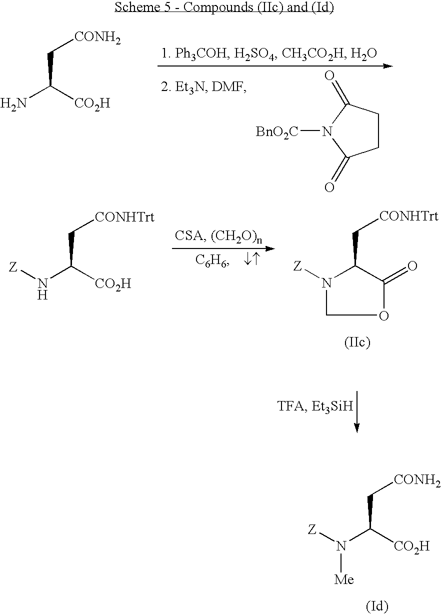

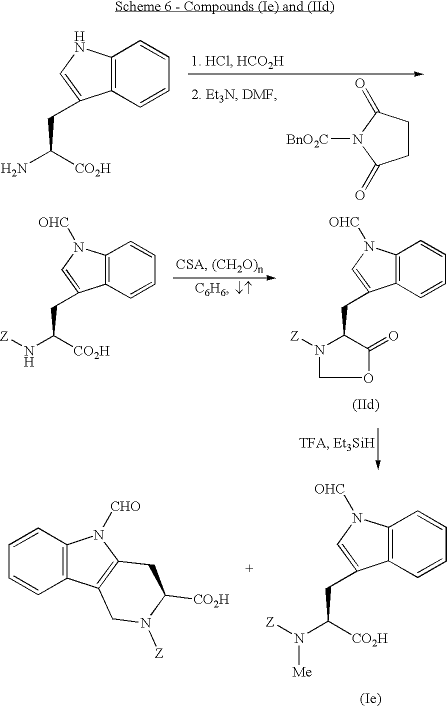

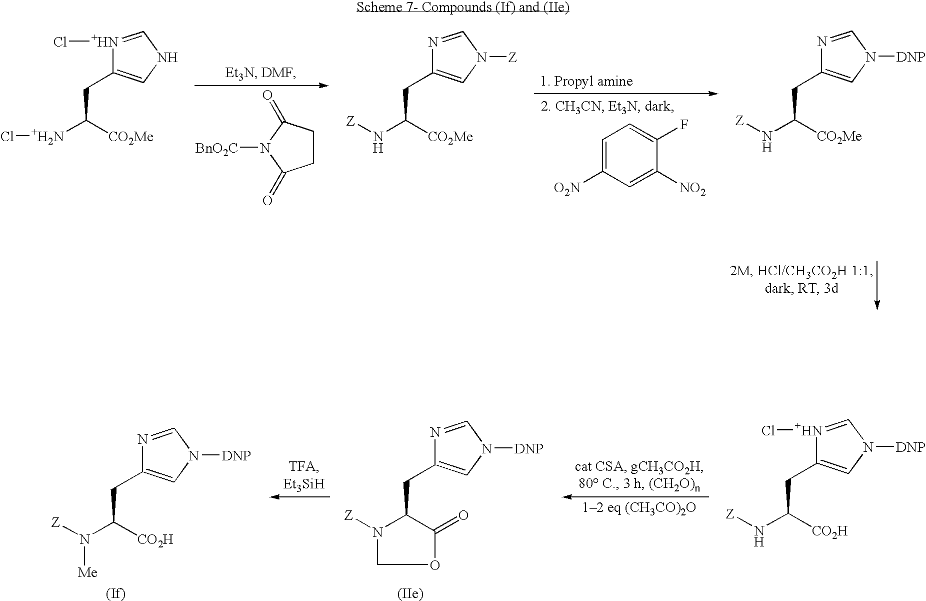

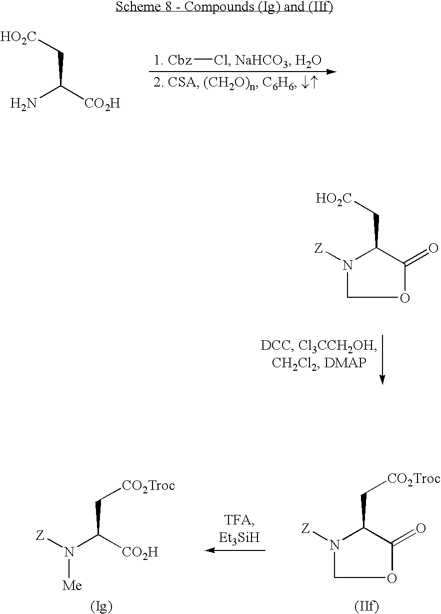

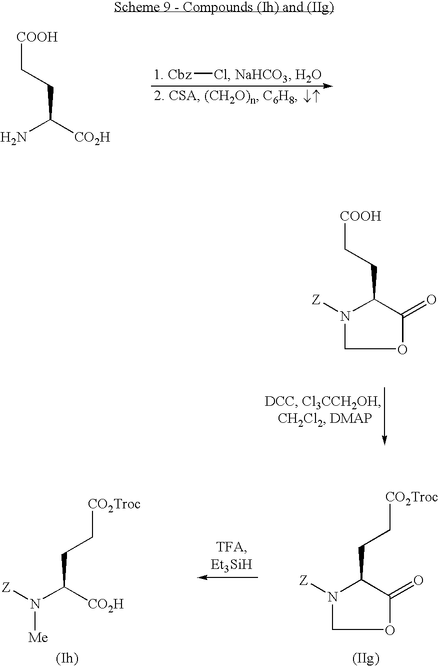

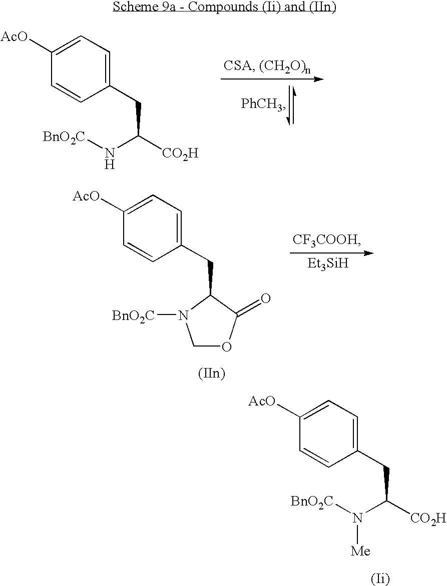

The term “peptide” is used herein in its broadest sense and refers to a compound formed by linking amino acids with amide bonds, using the amino group of one molecule and the carboxyl group in another. The peptide may be a dipeptide containing two amino acid residues, a tripeptide containing three amino acid residues and so on up to oligopeptides which contain relatively short chains of several amino acid residues and longer polymers which are polypeptides or proteins.

In one embodiment, the peptide is a dipeptide which bears an internal N-methyl amide bond of formula V: 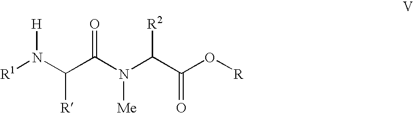
in which
R¹ and R² are as defined in formula I above, R′ is an optionally protected amino acid side chain and R is H or a carboxyl-protecting group.

This is deemed to be useful as it is known in peptide synthesis that the coupling efficiency of N-methyl residues to the carboxyl terminus of a growing peptide are not in the vicinity of 99.5% that is required to avoid deletion sequences. By manufacturing dipeptides that already have the N-methyl amide bond it is thought that potential users will be able, in particular, to program their incorporation into peptide sequences when using automated peptide assembly devices as monomeric species when in reality the entity incorporated is dimeric. The coupling reaction in this event will be a standard NH to COOH coupling and so ought to be in the region of 99.5% yield as the N-methyl bond has already been formed in the dipeptide. Examples of dipeptides of formula V are as follows:

| | MeGly-OR | MeAla-OR | MeVal-OR | MeLeu-OR |
|---|---|---|---|---|
| Z-Gly | Z-Gly-MeGly-OR | Z-Gly-MeAla-OR | Z-Gly-MeVal-OR | Z-Gly-MeLeu-OR |
| Z-Ala | Z-Ala-MeGly-OR | Z-Ala-MeAla-OR | Z-Ala-MeVal-OR | Z-Ala-MeLeu-OR |
| Z-Val | Z-Val-MeGly-OR | Z-Val-MeAla-OR | Z-Val-MeVal-OR | Z-Val-MeLeu-OR |
| Z-Leu | Z-Leu-MeGly-OR | Z-Leu-MeAla-OR | Z-Leu-MeVal-OR | Z-Leu-MeLeu-OR |
| Z-Ile | Z-Ile-MeGly-OR | Z-Ile-MeAla-OR | Z-Ile-MeVal-OR | Z-Ile-MeLeu-OR |
| Z-Phe | Z-Phe-MeGly-OR | Z-Phe-MeAla-OR | Z-Phe-MeVal-OR | Z-Phe-MeLeu-OR |
| Z-Tyr | Z-Tyr-MeGly-OR | Z-Tyr-MeAla-OR | Z-Tyr-MeVal-OR | Z-Tyr-MeLeu-OR |
| Z-Ser | Z-Ser-MeGly-OR | Z-Ser-MeAla-OR | Z-Ser-MeVal-OR | Z-Ser-MeLeu-OR |
| Z-Thr | Z-Thr-MeGly-OR | Z-Thr-MeAla-OR | Z-Thr-MeVal-OR | Z-Thr-MeLeu-OR |
| Z-Cys | Z-Cys-MeGly-OR | Z-Cys-MeAla-OR | Z-Cys-MeVal-OR | Z-Cys-MeLeu-OR |
| Z-Met | Z-Met-MeGly-OR | Z-Met-MeAla-OR | Z-Met-MeVal-OR | Z-Met-MeLeu-OR |
| Z-Asp | Z-Asp-MeGly-OR | Z-Asp-MeAla-OR | Z-Asp-MeVal-OR | Z-Asp-MeLeu-OR |
| Z-Glu | Z-Glu-MeGly-OR | Z-Glu-MeAla-OR | Z-Glu-MeVal-OR | Z-Glu-MeLeu-OR |
| Z-Asn | Z-Asn-MeGly-OR | Z-Asn-MeAla-OR | Z-Asn-MeVal-OR | Z-Asn-MeLeu-OR |
| Z-Gln | Z-Gln-MeGly-OR | Z-Gln-MeAla-OR | Z-Gln-MeVal-OR | Z-Gln-MeLeu-OR |
| Z-Lys | Z-Lys-MeGly-OR | Z-Lys-MeAla-OR | Z-Lys-MeVal-OR | Z-Lys-MeLeu-OR |
| Z-Trp | Z-Trp-MeGly-OR | Z-Trp-MeAla-OR | Z-Trp-MeVal-OR | Z-Trp-MeLeu-OR |
| Z-His | Z-His-MeGly-OR | Z-His-MeAla-OR | Z-His-MeVal-OR | Z-His-MeLeu-OR |
| Z-Arg | Z-Arg-MeGly-OR | Z-Arg-MeAla-OR | Z-Arg-MeVal-OR | Z-Arg-MeLeu-OR |
| Z-Pro | Z-Pro-MeGly-OR | Z-Pro-MeAla-OR | Z-Pro-MeVal-OR | Z-Pro-MeLeu-OR |
| Z-Orn | Z-Orn-MeGly-OR | Z-Orn-MeAla-Or | Z-Orn-MeVal-OR | Z-Orn-MeLeu-OR |

| | MeIle-OR | MePhe-OR | MeTyr-OR | MeSer-OR |
|---|---|---|---|---|
| Z-Gly | Z-Gly-MeIle-OR | Z-Gly-MePhe-OR | Z-Gly-MeTyr-OR | Z-Gly-MeSer-OR |
| Z-Ala | Z-Ala-MeIle-OR | Z-Ala-MePhe-OR | Z-Ala-MeTyr-OR | Z-Ala-MeSer-OR |
| Z-Val | Z-Val-MeIle-OR | Z-Val-MePhe-OR | Z-Val-MeTyr-OR | Z-Val-MeSer-OR |
| Z-Leu | Z-Leu-MeIle-OR | Z-Leu-MePhe-OR | Z-Leu-MeTyr-OR | Z-Leu-MeSer-OR |
| Z-Ile | Z-Ile-MeIle-OR | Z-Ile-MePhe-OR | Z-Ile-MeTyr-OR | Z-Ile-MeSer-OR |
| Z-Phe | Z-Phe-MeIle-OR | Z-Phe-MePhe-OR | Z-Phe-MeTyr-OR | Z-Phe-MeSer-OR |
| Z-Tyr | Z-Tyr-MeIle-OR | Z-Tyr-MePhe-OR | Z-Tyr-MeTyr-OR | Z-Tyr-MeSer-OR |
| Z-Ser | Z-Ser-MeIle-OR | Z-Ser-MePhe-OR | Z-Ser-MeTyr-OR | Z-Ser-MeSer-OR |
| Z-Thr | Z-Thr-MeIle-OR | Z-Thr-MePhe-OR | Z-Thr-MeTyr-OR | Z-Thr-MeSer-OR |
| Z-Cys | Z-Cys-MeIle-OR | Z-Cys-MePhe-OR | Z-Cys-MeTyr-OR | Z-Cys-MeSer-OR |
| Z-Met | Z-Met-MeIle-OR | Z-Met-MePhe-OR | Z-Met-MeTyr-OR | Z-Met-MeSer-OR |
| Z-Asp | Z-Asp-MeIle-OR | Z-Asp-MePhe-OR | Z-Asp-MeTyr-OR | Z-Asp-MeSer-OR |
| Z-Glu | Z-Glu-MeIle-OR | Z-Glu-MePhe-OR | Z-Glu-MeTyr-OR | Z-Glu-MeSer-OR |
| Z-Asn | Z-Asn-MeIle-OR | Z-Asn-MePhe-OR | Z-Asn-MeTyr-OR | Z-Asn-MeSer-OR |
| Z-Gln | Z-Gln-MeIle-OR | Z-Gln-MePhe-OR | Z-Gln-MeTyr-OR | Z-Gln-MeSer-OR |
| Z-Lys | Z-Lys-MeIle-OR | Z-Lys-MePhe-OR | Z-Lys-MeTyr-OR | Z-Lys-MeSer-OR |
| Z-Trp | Z-Trp-MeIle-OR | Z-Trp-MePhe-OR | Z-Trp-MeTyr-OR | Z-Trp-MeSer-OR |
| Z-His | Z-His-MeIle-OR | Z-His-MePhe-OR | Z-His-MeTyr-OR | Z-His-MeSer-OR |
| Z-Arg | Z-Arg-MeIle-OR | Z-Arg-MePhe-OR | Z-Arg-MeTyr-OR | Z-Arg-MeSer-OR |
| Z-Pro | Z-Pro-MeIle-OR | Z-Pro-MePhe-OR | Z-Pro-MeTyr-OR | Z-Pro-MeSer-OR |
| Z-Orn | Z-Orn-MeIle-OR | Z-Orn-MePhe-OR | Z-Orn-MeTyr-OR | Z-Orn-MeSer-OR |

| | MeThr-OR | MeCys-OR | MeMet-OR | MeAsp-OR |
|---|---|---|---|---|
| Z-Gly | Z-Gly-MeThr-OR | Z-Gly-MeCys-OR | Z-Gly-MeMet-OR | Z-Gly-MeAsp-OR |
| Z-Ala | Z-Ala-MeThr-OR | Z-Ala-MeCys-OR | Z-Ala-MeMet-OR | Z-Ala-MeAsp-OR |
| Z-Val | Z-Val-MeThr-OR | Z-Val-MeCys-OR | Z-Val-MeMet-OR | Z-Val-MeAsp-OR |
| Z-Leu | Z-Leu-MeThr-OR | Z-Leu-MeCys-OR | Z-Leu-MeMet-OR | Z-Leu-MeAsp-OR |
| Z-Ile | Z-Ile-MeThr-OR | Z-Ile-MeCys-OR | Z-Ile-MeMet-OR | Z-Ile-MeAsp-OR |
| Z-Phe | Z-Phe-MeThr-OR | Z-Phe-MeCys-OR | Z-Phe-MeMet-OR | Z-Phe-MeAsp-OR |
| Z-Tyr | Z-Tyr-MeThr-OR | Z-Tyr-MeCys-OR | Z-Tyr-MeMet-OR | Z-Tyr-MeAsp-OR |
| Z-Ser | Z-Ser-MeThr-OR | Z-Ser-MeCys-OR | Z-Ser-MeMet-OR | Z-Ser-MeAsp-OR |
| Z-Thr | Z-Thr-MeThr-OR | Z-Thr-MeCys-OR | Z-Thr-MeMet-OR | Z-Thr-MeAsp-OR |
| Z-Cys | Z-Cys-MeThr-OR | Z-Cys-MeCys-OR | Z-Cys-MeMet-OR | Z-Cys-MeAsp-OR |
| Z-Met | Z-Met-MeThr-OR | Z-Met-MeCys-OR | Z-Met-MeMet-OR | Z-Met-MeAsp-OR |
| Z-Asp | Z-Asp-MeThr-OR | Z-Asp-MeCys-OR | Z-Asp-MeMet-OR | Z-Asp-MeAsp-OR |
| Z-Glu | Z-Glu-MeThr-OR | Z-Glu-MeCys-OR | Z-Glu-MeMet-OR | Z-Glu-MeAsp-OR |
| Z-Asn | Z-Asn-MeThr-OR | Z-Asn-MeCys-OR | Z-Asn-MeMet-OR | Z-Asn-MeAsp-OR |
| Z-Gln | Z-Gln-MeThr-OR | Z-Gln-MeCys-OR | Z-Gln-MeMet-OR | Z-Gln-MeAsp-OR |
| Z-Lys | Z-Lys-MeThr-OR | Z-Lys-MeCys-OR | Z-Lys-MeMet-OR | Z-Lys-MeAsp-OR |
| Z-Trp | Z-Trp-MeThr-OR | Z-Trp-MeCys-OR | Z-Trp-MeMet-OR | Z-Trp-MeAsp-OR |
| Z-His | Z-His-MeThr-OR | Z-His-MeCys-OR | Z-His-MeMet-OR | Z-His-MeAsp-OR |
| Z-Arg | Z-Arg-MeThr-OR | Z-Arg-MeCys-OR | Z-Arg-MeMet-OR | Z-Arg-MeAsp-OR |
| Z-Pro | Z-Pro-MeThr-OR | Z-Pro-MeCys-OR | Z-Pro-MeMet-OR | Z-Pro-MeAsp-OR |
| Z-Orn | Z-Orn-MeThr-OR | Z-Orn-MeCys-OR | Z-Orn-MeMet-OR | Z-Orn-MeAsp-OR |

| | MeGlu-OR | MeAsn-OR | MeGln-OR | MeLys-OR |
|---|---|---|---|---|
| Z-Gly | Z-Gly-MeGlu-OR | Z-Gly-MeAsn-OR | Z-Gly-MeGln-OR | Z-Gly-MeLys-OR |
| Z-Ala | Z-Ala-MeGlu-OR | Z-Ala-MeAsn-OR | Z-Ala-MeGln-OR | Z-Ala-MeLys-OR |
| Z-Val | Z-Val-MeGlu-OR | Z-Val-MeAsn-OR | Z-Val-MeGln-OR | Z-Val-MeLys-OR |
| Z-Leu | Z-Leu-MeGlu-OR | Z-Leu-MeAsn-OR | Z-Leu-MeGln-OR | Z-Leu-MeLys-OR |
| Z-Ile | Z-Ile-MeGlu-OR | Z-Ile-MeAsn-OR | Z-Ile-MeGln-OR | Z-Ile-MeLys-OR |
| Z-Phe | Z-Phe-MeGlu-OR | Z-Phe-MeAsn-OR | Z-Phe-MeGln-OR | Z-Phe-MeLys-OR |
| Z-Tyr | Z-Tyr-MeGlu-OR | Z-Tyr-MeAsn-OR | Z-Tyr-MeGln-OR | Z-Tyr-MeLys-OR |
| Z-Ser | Z-Ser-MeGlu-OR | Z-Ser-MeAsn-OR | Z-Ser-MeGln-OR | Z-Ser-MeLys-OR |
| Z-Thr | Z-Thr-MeGlu-OR | Z-Thr-MeAsn-OR | Z-Thr-MeGln-OR | Z-Thr-MeLys-OR |
| Z-Cys | Z-Cys-MeGlu-OR | Z-Cys-MeAsn-OR | Z-Cys-MeGln-OR | Z-Cys-MeLys-OR |
| Z-Met | Z-Met-MeGlu-OR | Z-Met-MeAsn-OR | Z-Met-MeGln-OR | Z-Met-MeLys-OR |
| Z-Asp | Z-Asp-MeGlu-OR | Z-Asp-MeAsn-OR | Z-Asp-MeGln-OR | Z-Asp-MeLys-OR |
| Z-Glu | Z-Glu-MeGlu-OR | Z-Glu-MeAsn-OR | Z-Glu-MeGln-OR | Z-Glu-MeLys-OR |
| Z-Asn | Z-Asn-MeGlu-OR | Z-Asn-MeAsn-OR | Z-Asn-MeGln-OR | Z-Asn-MeLys-OR |
| Z-Gln | Z-Gln-MeGlu-OR | Z-Gln-MeAsn-OR | Z-Gln-MeGln-OR | Z-Gln-MeLys-OR |
| Z-Lys | Z-Lys-MeGlu-OR | Z-Lys-MeAsn-OR | Z-Lys-MeGln-OR | Z-Lys-MeLys-OR |
| Z-Trp | Z-Trp-MeGlu-OR | Z-Trp-MeAsn-OR | Z-Trp-MeGln-OR | Z-Trp-MeLys-OR |
| Z-His | Z-His-MeGlu-OR | Z-His-MeAsn-OR | Z-His-MeGln-OR | Z-His-MeLys-OR |
| Z-Arg | Z-Arg-MeGlu-OR | Z-Arg-MeAsn-OR | Z-Arg-MeGln-OR | Z-Arg-MeLys-OR |
| Z-Pro | Z-Pro-MeGlu-OR | Z-Pro-MeAsn-OR | Z-Pro-MeGln-OR | Z-Pro-MeLys-OR |
| Z-Orn | Z-Orn-MeGlu-OR | Z-Orn-MeAsn-OR | Z-Orn-MeGln-OR | Z-Orn-MeLys-OR |

| | MeTrp-OR | MeHis-OR | MeArg-OR | MeOrn-OR |
|---|---|---|---|---|
| Z-Gly | Z-Gly-MeTrp-OR | Z-Gly-MeHis-OR | Z-Gly-MeArg-OR | Z-Gly-MeOrn-OR |
| Z-Ala | Z-Ala-MeTrp-OR | Z-Ala-MeHis-OR | Z-Ala-MeArg-OR | Z-Ala-MeOrn-OR |
| Z-Val | Z-Val-MeTrp-OR | Z-Val-MeHis-OR | Z-Val-MeArg-OR | Z-Val-MeOrn-OR |
| Z-Leu | Z-Leu-MeTrp-OR | Z-Leu-MeHis-OR | Z-Leu-MeArg-OR | Z-Leu-MeOrn-OR |
| Z-Ile | Z-Ile-MeTrp-OR | Z-Ile-MeHis-OR | Z-Ile-MeArg-OR | Z-Ile-MeOrn-OR |
| Z-Phe | Z-Phe-MeTrp-OR | Z-Phe-MeHis-OR | Z-Phe-MeArg-OR | Z-Phe-MeOrn-OR |
| Z-Tyr | Z-Tyr-MeTrp-OR | Z-Tyr-MeHis-OR | Z-Tyr-MeArg-OR | Z-Tyr-MeOrn-OR |
| Z-Ser | Z-Ser-MeTrp-OR | Z-Ser-MeHis-OR | Z-Ser-MeArg-OR | Z-Ser-MeOrn-OR |
| Z-Thr | Z-Thr-MeTrp-OR | Z-Thr-MeHis-OR | Z-Thr-MeArg-OR | Z-Thr-MeOrn-OR |
| Z-Cys | Z-Cys-MeTrp-OR | Z-Cys-MeHis-OR | Z-Cys-MeArg-OR | Z-Cys-MeOrn-OR |
| Z-Met | Z-Met-MeTrp-OR | Z-Met-MeHis-OR | Z-Met-MeArg-OR | Z-Met-MeOrn-OR |
| Z-Asp | Z-Asp-MeTrp-OR | Z-Asp-MeHis-OR | Z-Asp-MeArg-OR | Z-Asp-MeOrn-OR |
| Z-Glu | Z-Glu-MeTrp-OR | Z-Glu-MeHis-OR | Z-Glu-MeArg-OR | Z-Glu-MeOrn-OR |
| Z-Asn | Z-Asn-MeTrp-OR | Z-Asn-MeHis-OR | Z-Asn-MeArg-OR | Z-Asn-MeOrn-OR |
| Z-Gln | Z-Gln-MeTrp-OR | Z-Gln-MeHis-OR | Z-Gln-MeArg-OR | Z-Gln-MeOrn-OR |
| Z-Lys | Z-Lys-MeTrp-OR | Z-Lys-MeHis-OR | Z-Lys-MeArg-OR | Z-Lys-MeOrn-OR |
| Z-Trp | Z-Trp-MeTrp-OR | Z-Trp-MeHis-OR | Z-Trp-MeArg-OR | Z-Trp-MeOrn-OR |
| Z-His | Z-His-MeTrp-OR | Z-His-MeHis-OR | Z-His-MeArg-OR | Z-His-MeOrn-OR |
| Z-Arg | Z-Arg-MeTrp-OR | Z-Arg-MeHis-OR | Z-Arg-MeArg-OR | Z-Arg-MeOrn-OR |
| Z-Pro | Z-Pro-MeTrp-OR | Z-Pro-MeHis-OR | Z-Pro-MeArg-OR | Z-Pro-MeOrn-OR |
| Z-Orn | Z-Orn-MeTrp-OR | Z-Orn-MeHis-OR | Z-Orn-MeArg-OR | Z-Orn-MeOrn-OR |

| | MeGly-OR | MeAla-OR | MeVal-OR | MeLeu-OR |
|---|---|---|---|---|
| Fmoc-Gly | Fmoc-Gly-MeGly-OR | Fmoc-Gly-MeAla-OR | Fmoc-Gly-MeVal-OR | Fmoc-Gly-MeLeu-OR |
| Fmoc-Ala | Fmoc-Ala-MeGly-OR | Fmoc-Ala-MeAla-OR | Fmoc-Ala-MeVal-OR | Fmoc-Ala-MeLeu-OR |
| Fmoc-Val | Fmoc-Val-MeGly-OR | Fmoc-Val-MeAla-OR | Fmoc-Val-MeVal-OR | Fmoc-Val-MeLeu-OR |
| Fmoc-Leu | Fmoc-Leu-MeGly-OR | Fmoc-Leu-MeAla-OR | Fmoc-Leu-MeVal-OR | Fmoc-Leu-MeLeu-OR |
| Fmoc-Ile | Fmoc-Ile-MeGly-OR | Fmoc-Ile-MeAla-OR | Fmoc-Ile-MeVal-OR | Fmoc-Ile-MeLeu-OR |
| Fmoc-Phe | Fmoc-Phe-MeGly-OR | Fmoc-Phe-MeAla-OR | Fmoc-Phe-MeVal-OR | Fmoc-Phe-MeLeu-OR |
| Fmoc-Tyr | Fmoc-Tyr-MeGly-OR | Fmoc-Tyr-MeAla-OR | Fmoc-Tyr-MeVal-OR | Fmoc-Tyr-MeLeu-OR |
| Fmoc-Ser | Fmoc-Ser-MeGly-OR | Fmoc-Ser-MeAla-OR | Fmoc-Ser-MeVal-OR | Fmoc-Ser-MeLeu-OR |
| Fmoc-Thr | Fmoc-Thr-MeGly-OR | Fmoc-Thr-MeAla-OR | Fmoc-Thr-MeVal-OR | Fmoc-Thr-MeLeu-OR |
| Fmoc-Cys | Fmoc-Cys-MeGly-OR | Fmoc-Cys-MeAla-OR | Fmoc-Cys-MeVal-OR | Fmoc-Cys-MeLeu-OR |
| Fmoc-Met | Fmoc-Met-MeGly-OR | Fmoc-Met-MeAla-OR | Fmoc-Met-MeVal-OR | Fmoc-Met-MeLeu-OR |
| Fmoc-Asp | Fmoc-Asp-MeGly-OR | Fmoc-Asp-MeAla-OR | Fmoc-Asp-MeVal-OR | Fmoc-Asp-MeLeu-OR |
| Fmoc-Glu | Fmoc-Glu-MeGly-OR | Fmoc-Glu-MeAla-OR | Fmoc-Glu-MeVal-OR | Fmoc-Glu-MeLeu-OR |
| Fmoc-Asn | Fmoc-Asn-MeGly-OR | Fmoc-Asn-MeAla-OR | Fmoc-Asn-MeVal-OR | Fmoc-Asn-MeLeu-OR |
| Fmoc-Gln | Fmoc-Gln-MeGly-OR | Fmoc-Gln-MeAla-OR | Fmoc-Gln-MeVal-OR | Fmoc-Gln-MeLeu-OR |
| Fmoc-Lys | Fmoc-Lys-MeGly-OR | Fmoc-Lys-MeAla-OR | Fmoc-Lys-MeVal-OR | Fmoc-Lys-MeLeu-OR |
| Fmoc-Trp | Fmoc-Trp-MeGly-OR | Fmoc-Trp-MeAla-OR | Fmoc-Trp-MeVal-OR | Fmoc-Trp-MeLeu-OR |
| Fmoc-His | Fmoc-His-MeGly-OR | Fmoc-His-MeAla-OR | Fmoc-His-MeVal-OR | Fmoc-His-MeLeu-OR |
| Fmoc-Arg | Fmoc-Arg-MeGly-OR | Fmoc-Arg-MeAla-OR | Fmoc-Arg-MeVal-OR | Fmoc-Arg-MeLeu-OR |
| Fmoc-Pro | Fmoc-Pro-MeGly-OR | Fmoc-Pro-MeAla-OR | Fmoc-Pro-MeVal-OR | Fmoc-Pro-MeLeu-OR |
| Fmoc-Orn | Fmoc-Orn-MeGly-OR | Fmoc-Orn-MeAla-OR | Fmoc-Orn-MeVal-OR | Fmoc-Orn-MeLeu-OR |

| | MeIle-OR | MePhe-OR | MeTyr-OR |
|---|---|---|---|
| Fmoc-Gly | Fmoc-Gly-MeIle-OR | Fmoc-Gly-MePhe-OR | Fmoc-Gly-MeTyr-OR |
| Fmoc-Ala | Fmoc-Ala-MeIle-OR | Fmoc-Ala-MePhe-OR | Fmoc-Ala-MeTyr-OR |
| Fmoc-Val | Fmoc-Val-MeIle-OR | Fmoc-Val-MePhe-OR | Fmoc-Val-MeTyr-OR |
| Fmoc-Leu | Fmoc-Leu-MeIle-OR | Fmoc-Leu-MePhe-OR | Fmoc-Leu-MeTyr-OR |
| Fmoc-Ile | Fmoc-Ile-MeIle-OR | Fmoc-Ile-MePhe-OR | Fmoc-Ile-MeTyr-OR |
| Fmoc-Phe | Fmoc-Phe-MeIle-OR | Fmoc-Phe-MePhe-OR | Fmoc-Phe-MeTyr-OR |
| Fmoc-Tyr | Fmoc-Tyr-MeIle-OR | Fmoc-Tyr-MePhe-OR | Fmoc-Tyr-MeTyr-OR |
| Fmoc-Ser | Fmoc-Ser-MeIle-OR | Fmoc-Ser-MePhe-OR | Fmoc-Ser-MeTyr-OR |
| Fmoc-Thr | Fmoc-Thr-MeIle-OR | Fmoc-Thr-MePhe-OR | Fmoc-Thr-MeTyr-OR |
| Fmoc-Cys | Fmoc-Cys-MeIle-OR | Fmoc-Cys-MePhe-OR | Fmoc-Cys-MeTyr-OR |
| Fmoc-Met | Fmoc-Met-MeIle-OR | Fmoc-Met-MePhe-OR | Fmoc-Met-MeTyr-OR |
| Fmoc-Asp | Fmoc-Asp-MeIle-OR | Fmoc-Asp-MePhe-OR | Fmoc-Asp-MeTyr-OR |
| Fmoc-Glu | Fmoc-Glu-MeIle-OR | Fmoc-Glu-MePhe-OR | Fmoc-Glu-MeTyr-OR |
| Fmoc-Asn | Fmoc-Asn-MeIle-OR | Fmoc-Asn-MePhe-OR | Fmoc-Asn-MeTyr-OR |
| Fmoc-Gln | Fmoc-Gln-MeIle-OR | Fmoc-Gln-MePhe-OR | Fmoc-Gln-MeTyr-OR |
| Fmoc-Lys | Fmoc-Lys-MeIle-OR | Fmoc-Lys-MePhe-OR | Fmoc-Lys-MeTyr-OR |
| Fmoc-Trp | Fmoc-Trp-MeIle-OR | Fmoc-Trp-MePhe-OR | Fmoc-Trp-MeTyr-OR |
| Fmoc-His | Fmoc-His-MeIle-OR | Fmoc-His-MePhe-OR | Fmoc-His-MeTyr-OR |
| Fmoc-Arg | Fmoc-Arg-MeIle-OR | Fmoc-Arg-MePhe-OR | Fmoc-Arg-MeTyr-OR |
| Fmoc-Pro | Fmoc-Pro-MeIle-OR | Fmoc-Pro-MePhe-OR | Fmoc-Pro-MeTyr-OR |
| Fmoc-Orn | Fmoc-Orn-MeIle-OR | Fmoc-Orn-MePhe-OR | Fmoc-Orn-MeTyr-OR |

| | MeSer-OR | MeThr-OR | MeCys-OR | MeMet-OR |
|---|---|---|---|---|
| Fmoc-Gly | Fmoc-Gly-MeSer-OR | Fmoc-Gly-MeThr-OR | Fmoc-Gly-MeCys-OR | Fmoc-Gly-MeMet-OR |
| Fmoc-Ala | Fmoc-Ala-MeSer-OR | Fmoc-Ala-MeThr-OR | Fmoc-Ala-MeCys-OR | Fmoc-Ala-MeMet-OR |
| Fmoc-Val | Fmoc-Val-MeSer-OR | Fmoc-Val-MeThr-OR | Fmoc-Val-MeCys-OR | Fmoc-Val-MeMet-OR |
| Fmoc-Leu | Fmoc-Leu-MeSer-OR | Fmoc-Leu-MeThr-OR | Fmoc-Leu-MeCys-OR | Fmoc-Leu-MeMet-OR |
| Fmoc-Ile | Fmoc-Ile-MeSer-OR | Fmoc-Ile-MeThr-OR | Fmoc-Ile-MeCys-OR | Fmoc-Ile-MeMet-OR |
| Fmoc-Phe | Fmoc-Phe-MeSer-OR | Fmoc-Phe-MeThr-OR | Fmoc-Phe-MeCys-OR | Fmoc-Phe-MeMet-OR |
| Fmoc-Tyr | Fmoc-Tyr-MeSer-OR | Fmoc-Tyr-MeThr-OR | Fmoc-Tyr-MeCys-OR | Fmoc-Tyr-MeMet-OR |
| Fmoc-Ser | Fmoc-Ser-MeSer-OR | Fmoc-Ser-MeThr-OR | Fmoc-Ser-MeCys-OR | Fmoc-Ser-MeMet-OR |
| Fmoc-Thr | Fmoc-Thr-MeSer-OR | Fmoc-Thr-MeThr-OR | Fmoc-Thr-MeCys-OR | Fmoc-Thr-MeMet-OR |
| Fmoc-Cys | Fmoc-Cys-MeSer-OR | Fmoc-Cys-MeThr-OR | Fmoc-Cys-MeCys-OR | Fmoc-Cys-MeMet-OR |
| Fmoc-Met | Fmoc-Met-MeSer-OR | Fmoc-Met-MeThr-OR | Fmoc-Met-MeCys-OR | Fmoc-Met-MeMet-OR |
| Fmoc-Asp | Fmoc-Asp-MeSer-OR | Fmoc-Asp-MeThr-OR | Fmoc-Asp-MeCys-OR | Fmoc-Asp-MeMet-OR |
| Fmoc-Glu | Fmoc-Glu-MeSer-OR | Fmoc-Glu-MeThr-OR | Fmoc-Glu-MeCys-OR | Fmoc-Glu-MeMet-OR |
| Fmoc-Asn | Fmoc-Asn-MeSer-OR | Fmoc-Asn-MeThr-OR | Fmoc-Asn-MeCys-OR | Fmoc-Asn-MeMet-OR |
| Fmoc-Gln | Fmoc-Gln-MeSer-OR | Fmoc-Gln-MeThr-OR | Fmoc-Gln-MeCys-OR | Fmoc-Gln-MeMet-OR |
| Fmoc-Lys | Fmoc-Lys-MeSer-OR | Fmoc-Lys-MeThr-OR | Fmoc-Lys-MeCys-OR | Fmoc-Lys-MeMet-OR |
| Fmoc-Trp | Fmoc-Trp-MeSer-OR | Fmoc-Trp-MeThr-OR | Fmoc-Trp-MeCys-OR | Fmoc-Trp-MeMet-OR |
| Fmoc-His | Fmoc-His-MeSer-OR | Fmoc-His-MeThr-OR | Fmoc-His-MeCys-OR | Fmoc-His-MeMet-OR |
| Fmoc-Arg | Fmoc-Arg-MeSer-OR | Fmoc-Arg-MeThr-OR | Fmoc-Arg-MeCys-OR | Fmoc-Arg-MeMet-OR |
| Fmoc-Pro | Fmoc-Pro-MeSer-OR | Fmoc-Pro-MeThr-OR | Fmoc-Pro-MeCys-OR | Fmoc-Pro-MeMet-OR |
| Fmoc-Orn | Fmoc-Orn-MeSer-OR | Fmoc-Orn-MeThr-OR | Fmoc-Orn-MeCys-OR | Fmoc-Orn-MeMet-OR |

| | MeAsp-OR | MeGlu-OR | MeAsn-OR |
|---|---|---|---|
| Fmoc-Gly | Fmoc-Gly-MeAsp-OR | Fmoc-Gly-MeGlu-OR | Fmoc-Gly-MeAsn-OR |
| Fmoc-Ala | Fmoc-Ala-MeAsp-OR | Fmoc-Ala-MeGlu-OR | Fmoc-Ala-MeAsn-OR |
| Fmoc-Val | Fmoc-Val-MeAsp-OR | Fmoc-Val-MeGlu-OR | Fmoc-Val-MeAsn-OR |
| Fmoc-Leu | Fmoc-Leu-MeAsp-OR | Fmoc-Leu-MeGlu-OR | Fmoc-Leu-MeAsn-OR |
| Fmoc-Ile | Fmoc-Ile-MeAsp-OR | Fmoc-Ile-MeGlu-OR | Fmoc-Ile-MeAsn-OR |
| Fmoc-Phe | Fmoc-Phe-MeAsp-OR | Fmoc-Phe-MeGlu-OR | Fmoc-Phe-MeAsn-OR |
| Fmoc-Tyr | Fmoc-Tyr-MeAsp-OR | Fmoc-Tyr-MeGlu-OR | Fmoc-Tyr-MeAsn-OR |
| Fmoc-Ser | Fmoc-Ser-MeAsp-OR | Fmoc-Ser-MeGlu-OR | Fmoc-Ser-MeAsn-OR |
| Fmoc-Thr | Fmoc-Thr-MeAsp-OR | Fmoc-Thr-MeGlu-OR | Fmoc-Thr-MeAsn-OR |
| Fmoc-Cys | Fmoc-Cys-MeAsp-OR | Fmoc-Cys-MeGlu-OR | Fmoc-Cys-MeAsn-OR |
| Fmoc-Met | Fmoc-Met-MeAsp-OR | Fmoc-Met-MeGlu-OR | Fmoc-Met-MeAsn-OR |
| Fmoc-Asp | Fmoc-Asp-MeAsp-OR | Fmoc-Asp-MeGlu-OR | Fmoc-Asp-MeAsn-OR |
| Fmoc-Glu | Fmoc-Glu-MeAsp-OR | Fmoc-Glu-MeGlu-OR | Fmoc-Glu-MeAsn-OR |
| Fmoc-Asn | Fmoc-Asn-MeAsp-OR | Fmoc-Asn-MeGlu-OR | Fmoc-Asn-MeAsn-OR |
| Fmoc-Gln | Fmoc-Gln-MeAsp-OR | Fmoc-Gln-MeGlu-OR | Fmoc-Gln-MeAsn-OR |
| Fmoc-Lys | Fmoc-Lys-MeAsp-OR | Fmoc-Lys-MeGlu-OR | Fmoc-Lys-MeAsn-OR |
| Fmoc-Trp | Fmoc-Trp-MeAsp-OR | Fmoc-Trp-MeGlu-OR | Fmoc-Trp-MeAsn-OR |
| Fmoc-His | Fmoc-His-MeAsp-OR | Fmoc-His-MeGlu-OR | Fmoc-His-MeAsn-OR |
| Fmoc-Arg | Fmoc-Arg-MeAsp-OR | Fmoc-Arg-MeGlu-OR | Fmoc-Arg-MeAsn-OR |
| Fmoc-Pro | Fmoc-Pro-MeAsp-OR | Fmoc-Pro-MeGlu-OR | Fmoc-Pro-MeAsn-OR |
| Fmoc-Orn | Fmoc-Orn-MeAsp-OR | Fmoc-Orn-MeGlu-OR | Fmoc-Orn-MeAsn-OR |
| | MeGln-OR | MeLys-OR | MeTrp-OR |
| Fmoc-Gly | Fmoc-Gly-MeGln-OR | Fmoc-Gly-MeLys-OR | Fmoc-Gly-MeTrp-OR |
| Fmoc-Ala | Fmoc-Ala-MeGln-OR | Fmoc-Ala-MeLys-OR | Fmoc-Ala-MeTrp-OR |
| Fmoc-Val | Fmoc-Val-MeGln-OR | Fmoc-Val-MeLys-OR | Fmoc-Val-MeTrp-OR |
| Fmoc-Leu | Fmoc-Leu-MeGln-OR | Fmoc-Leu-MeLys-OR | Fmoc-Leu-MeTrp-OR |
| Fmoc-Ile | Fmoc-Ile-MeGln-OR | Fmoc-Ile-MeLys-OR | Fmoc-Ile-MeTrp-OR |
| Fmoc-Phe | Fmoc-Phe-MeGln-OR | Fmoc-Phe-MeLys-OR | Fmoc-Phe-MeTrp-OR |
| Fmoc-Tyr | Fmoc-Tyr-MeGln-OR | Fmoc-Tyr-MeLys-OR | Fmoc-Tyr-MeTrp-OR |
| Fmoc-Ser | Fmoc-Ser-MeGln-OR | Fmoc-Ser-MeLys-OR | Fmoc-Ser-MeTrp-OR |
| Fmoc-Thr | Fmoc-Thr-MeGln-OR | Fmoc-Thr-MeLys-OR | Fmoc-Thr-MeTrp-OR |
| Fmoc-Cys | Fmoc-Cys-MeGln-OR | Fmoc-Cys-MeLys-OR | Fmoc-Cys-MeTrp-OR |
| Fmoc-Met | Fmoc-Met-MeGln-OR | Fmoc-Met-MeLys-OR | Fmoc-Met-MeTrp-OR |
| Fmoc-Asp | Fmoc-Asp-MeGln-OR | Fmoc-Asp-MeLys-OR | Fmoc-Asp-MeTrp-OR |
| Fmoc-Glu | Fmoc-Glu-MeGln-OR | Fmoc-Glu-MeLys-OR | Fmoc-Glu-MeTrp-OR |
| Fmoc-Asn | Fmoc-Asn-MeGln-OR | Fmoc-Asn-MeLys-OR | Fmoc-Asn-MeTrp-OR |
| Fmoc-Gln | Fmoc-Gln-MeGln-OR | Fmoc-Gln-MeLys-OR | Fmoc-Gln-MeTrp-OR |
| Fmoc-Lys | Fmoc-Lys-MeGln-OR | Fmoc-Lys-MeLys-OR | Fmoc-Lys-MeTrp-OR |
| Fmoc-Trp | Fmoc-Trp-MeGln-OR | Fmoc-Trp-MeLys-OR | Fmoc-Trp-MeTrp-OR |
| Fmoc-His | Fmoc-His-MeGln-OR | Fmoc-His-MeLys-OR | Fmoc-His-MeTrp-OR |
| Fmoc-Arg | Fmoc-Arg-MeGln-OR | Fmoc-Arg-MeLys-OR | Fmoc-Arg-MeTrp-OR |
| Fmoc-Pro | Fmoc-Pro-MeGln-OR | Fmoc-Pro-MeLys-OR | Fmoc-Pro-MeTrp-OR |
| Fmoc-Orn | Fmoc-Orn-MeGln-OR | Fmoc-Orn-MeLys-OR | Fmoc-Orn-MeTrp-OR |
| | MeHis-OR | MeArg-OR | MeOrn-OR |
| Fmoc-Gly | Fmoc-Gly-MeHis-OR | Fmoc-Gly-MeArg-OR | Fmoc-Gly-MeOrn-OR |
| Fmoc-Ala | Fmoc-Ala-MeHis-OR | Fmoc-Ala-MeArg-OR | Fmoc-Ala-MeOrn-OR |
| Fmoc-Val | Fmoc-Val-MeHis-OR | Fmoc-Val-MeArg-OR | Fmoc-Val-MeOrn-OR |
| Fmoc-Leu | Fmoc-Leu-MeHis-OR | Fmoc-Leu-MeArg-OR | Fmoc-Leu-MeOrn-OR |
| Fmoc-Ile | Fmoc-Ile-MeHis-OR | Fmoc-Ile-MeArg-OR | Fmoc-Ile-MeOrn-OR |
| Fmoc-Phe | Fmoc-Phe-MeHis-OR | Fmoc-Phe-MeArg-OR | Fmoc-Phe-MeOrn-OR |
| Fmoc-Tyr | Fmoc-Tyr-MeHis-OR | Fmoc-Tyr-MeArg-OR | Fmoc-Tyr-MeOrn-OR |
| Fmoc-Ser | Fmoc-Ser-MeHis-OR | Fmoc-Ser-MeArg-OR | Fmoc-Ser-MeOrn-OR |
| Fmoc-Thr | Fmoc-Thr-MeHis-OR | Fmoc-Thr-MeArg-OR | Fmoc-Thr-MeOrn-OR |
| Fmoc-Cys | Fmoc-Cys-MeHis-OR | Fmoc-Cys-MeArg-OR | Fmoc-Cys-MeOrn-OR |
| Fmoc-Met | Fmoc-Met-MeHis-OR | Fmoc-Met-MeArg-OR | Fmoc-Met-MeOrn-OR |
| Fmoc-Asp | Fmoc-Asp-MeHis-OR | Fmoc-Asp-MeArg-OR | Fmoc-Asp-MeOrn-OR |
| Fmoc-Glu | Fmoc-Glu-MeHis-OR | Fmoc-Glu-MeArg-OR | Fmoc-Glu-MeOrn-OR |
| Fmoc-Asn | Fmoc-Asn-MeHis-OR | Fmoc-Asn-MeArg-OR | Fmoc-Asn-MeOrn-OR |
| Fmoc-Gln | Fmoc-Gln-MeHis-OR | Fmoc-Gln-MeArg-OR | Fmoc-Gln-MeOrn-OR |
| Fmoc-Lys | Fmoc-Lys-MeHis-OR | Fmoc-Lys-MeArg-OR | Fmoc-Lys-MeOrn-OR |
| Fmoc-Trp | Fmoc-Trp MeHis-OR | Fmoc-Trp-MeArg-OR | Fmoc-Trp-MeOrn-OR |
| Fmoc-His | Fmoc-His-MeHis-OR | Fmoc-His-MeArg-OR | Fmoc-His-MeOrn-OR |
| Fmoc-Arg | Fmoc-Arg-MeHis-OR | Fmoc-Arg-MeArg-OR | Fmoc-Arg-MeOrn-OR |
| Fmoc-Pro | Fmoc-Pro-MeHis-OR | Fmoc-Pro-MeArg-OR | Fmoc-Pro-MeOrn-OR |
| Fmoc-Orn | Fmoc-Orn-MeHis-OR | Fmoc-Orn-MeArg-OR | Fmoc-Orn-MeOrn-OR |

| | MeGly-OR | MeAla-OR | MeVal-OR | MeLeu-OR |
|---|---|---|---|---|
| Boc-Gly | Boc-Gly-MeGly-OR | Boc-Gly-MeAla-OR | Boc-Gly-MeVal-OR | Boc-Gly-MeLeu-OR |
| Boc-Ala | Boc-Ala-MeGly-OR | Boc-Ala-MeAla-OR | Boc-Ala-MeVal-OR | Boc-Ala-MeLeu-OR |
| Boc-Val | Boc-Val-MeGly-OR | Boc-Val-MeAla-OR | Boc-Val-MeVal-OR | Boc-Val-MeLeu-OR |
| Boc-Leu | Boc-Leu-MeGly-OR | Boc-Leu-MeAla-OR | Boc-Leu-MeVal-OR | Boc-Leu-MeLeu-OR |
| Boc-Ile | Boc-Ile-MeGly-OR | Boc-Ile-MeAla-OR | Boc-Ile-MeVal-OR | Boc-Ile-MeLeu-OR |
| Boc-Phe | Boc-Phe-MeGly-OR | Boc-Phe-MeAla-OR | Boc-Phe-MeVal-OR | Boc-Phe-MeLeu-OR |
| Boc-Tyr | Boc-Tyr-MeGly-OR | Boc-Tyr-MeAla-OR | Boc-Tyr-MeVal-OR | Boc-Tyr-MeLeu-OR |
| Boc-Ser | Boc-Ser-MeGly-OR | Boc-Ser-MeAla-OR | Boc-Ser-MeVal-OR | Boc-Ser-MeLeu-OR |
| Boc-Thr | Boc-Thr-MeGly-OR | Boc-Thr-MeAla-OR | Boc-Thr-MeVal-OR | Boc-Thr-MeLeu-OR |
| Boc-Cys | Boc-Cys-MeGly-OR | Boc-Cys-MeAla-OR | Boc-Cys-MeVal-OR | Boc-Cys-MeLeu-OR |
| Boc-Met | Boc-Met-MeGly-OR | Boc-Met-MeAla-OR | Boc-Met-MeVal-OR | Boc-Met-MeLeu-OR |
| Boc-Asp | Boc-Asp-MeGly-OR | Boc-Asp-MeAla-OR | Boc-Asp-MeVal-OR | Boc-Asp-MeLeu-OR |
| Boc-Glu | Boc-Glu-MeGly-OR | Boc-Glu-MeAla-OR | Boc-Glu-MeVal-OR | Boc-Glu-MeLeu-OR |
| Boc-Asn | Boc-Asn-MeGly-OR | Boc-Asn-MeAla-OR | Boc-Asn-MeVal-OR | Boc-Asn-MeLeu-OR |
| Boc-Gln | Boc-Gln-MeGly-OR | Boc-Gln-MeAla-OR | Boc-Gln-MeVal-OR | Boc-Gln-MeLeu-OR |
| Boc-Lys | Boc-Lys-MeGly-OR | Boc-Lys-MeAla-OR | Boc-Lys-MeVal-OR | Boc-Lys-MeLeu-OR |
| Boc-Trp | Boc-Trp-MeGly-OR | Boc-Trp-MeAla-OR | Boc-Trp-MeVal-OR | Boc-Trp-MeLeu-OR |
| Boc-His | Boc-His-MeGly-OR | Boc-His-MeAla-OR | Boc-His-MeVal-OR | Boc-His-MeLeu-OR |
| Boc-Arg | Boc-Arg-MeGly-OR | Boc-Arg-MeAla-OR | Boc-Arg-MeVal-OR | Boc-Arg-MeLeu-OR |
| Boc-Pro | Boc-Pro-MeGly-OR | Boc-Pro-MeAla-OR | Boc-Pro-MeVal-OR | Boc-Pro-MeLeu-OR |
| Boc-Orn | Boc-Orn-MeGly-OR | Boc-Orn-MeAla-OR | Boc-Orn-MeVal-OR | Boc-Orn-MeLeu-OR |

| | MeIle-OR | MePhe-OR | MeTyr-OR |
|---|---|---|---|
| Boc-Gly | Boc-Gly-MeIle-OR | Boc-Gly-MePhe-OR | Boc-Gly-MeTyr-OR |
| Boc-Ala | Boc-Ala-MeIle-OR | Boc-Ala-MePhe-OR | Boc-Ala-MeTyr-OR |
| Boc-Val | Boc-Val-MeIle-OR | Boc-Val-MePhe-OR | Boc-Val-MeTyr-OR |
| Boc-Leu | Boc-Leu-MeIle-OR | Boc-Leu-MePhe-OR | Boc-Leu-MeTyr-OR |
| Boc-Ile | Boc-Ile-MeIle-OR | Boc-Ile-MePhe-OR | Boc-Ile-MeTyr-OR |
| Boc-Phe | Boc-Phe-MeIle-OR | Boc-Phe-MePhe-OR | Boc-Phe-MeTyr-OR |
| Boc-Tyr | Boc-Tyr-MeIle-OR | Boc-Tyr-MePhe-OR | Boc-Tyr-MeTyr-OR |
| Boc-Ser | Boc-Ser-MeIle-OR | Boc-Ser-MePhe-OR | Boc-Ser-MeTyr-OR |
| Boc-Thr | Boc-Thr-MeIle-OR | Boc-Thr-MePhe-OR | Boc-Thr-MeTyr-OR |
| Boc-Cys | Boc-Cys-MeIle-OR | Boc-Cys-MePhe-OR | Boc-Cys-MeTyr-OR |
| Boc-Met | Boc-Met-MeIle-OR | Boc-Met-MePhe-OR | Boc-Met-MeTyr-OR |
| Boc-Asp | Boc-Asp-MeIle-OR | Boc-Asp-MePhe-OR | Boc-Asp-MeTyr-OR |
| Boc-Glu | Boc-Glu-MeIle-OR | Boc-Glu-MePhe-OR | Boc-Glu-MeTyr-OR |
| Boc-Asn | Boc-Asn-MeIle-OR | Boc-Asn-MePhe-OR | Boc-Asn-MeTyr-OR |
| Boc-Gln | Boc-Gln-MeIle-OR | Boc-Gln-MePhe-OR | Boc-Gln-MeTyr-OR |
| Boc-Lys | Boc-Lys-MeIle-OR | Boc-Lys-MePhe-OR | Boc-Lys-MeTyr-OR |
| Boc-Trp | Boc-Trp-MeIle-OR | Boc-Trp-MePhe-OR | Boc-Trp-MeTyr-OR |
| Boc-His | Boc-His-MeIle-OR | Boc-His-MePhe-OR | Boc-His-MeTyr-OR |
| Boc-Arg | Boc-Arg-MeIle-OR | Boc-Arg-MePhe-OR | Boc-Arg-MeTyr-OR |
| Boc-Pro | Boc-Pro-MeIle-OR | Boc-Pro-MePhe-OR | Boc-Pro-MeTyr-OR |
| Boc-Orn | Boc-Orn-MeIle-OR | Boc-Orn-MePhe-OR | Boc-Orn-MeTyr-OR |

| | MeSer-OR | MeThr-OR | MeCys-OR | MeMet-OR |
|---|---|---|---|---|
| Boc-Gly | Boc-Gly-MeSer-OR | Boc-Gly-MeThr-OR | Boc-Gly-MeCys-OR | Boc-Gly-MeMet-OR |
| Boc-Ala | Boc-Ala-MeSer-OR | Boc-Ala-MeThr-OR | Boc-Ala-MeCys-OR | Boc-Ala-MeMet-OR |
| Boc-Val | Boc-Val-MeSer-OR | Boc-Val-MeThr-OR | Boc-Val-MeCys-OR | Boc-Val-MeMet-OR |
| Boc-Leu | Boc-Leu-MeSer-OR | Boc-Leu-MeThr-OR | Boc-Leu-MeCys-OR | Boc-Leu-MeMet-OR |
| Boc-Ile | Boc-Ile-MeSer-OR | Boc-Ile-MeThr-OR | Boc-Ile-MeCys-OR | Boc-Ile-MeMet-OR |
| Boc-Phe | Boc-Phe-MeSer-OR | Boc-Phe-MeThr-OR | Boc-Phe-MeCys-OR | Boc-Phe-MeMet-OR |
| Boc-Tyr | Boc-Tyr-MeSer-OR | Boc-Tyr-MeThr-OR | Boc-Tyr-MeCys-OR | Boc-Tyr-MeMet-OR |
| Boc-Ser | Boc-Ser-MeSer-OR | Boc-Ser-MeThr-OR | Boc-Ser-MeCys-OR | Boc-Ser-MeMet-OR |
| Boc-Thr | Boc-Thr-MeSer-OR | Boc-Thr-MeThr-OR | Boc-Thr-MeCys-OR | Boc-Thr-MeMet-OR |
| Boc-Cys | Boc-Cys-MeSer-OR | Boc-Cys-MeThr-OR | Boc-Cys-MeCys-OR | Boc-Cys-MeMet-OR |
| Boc-Met | Boc-Met-MeSer-OR | Boc-Met-MeThr-OR | Boc-Met-MeCys-OR | Boc-Met-MeMet-OR |
| Boc-Asp | Boc-Asp-MeSer-OR | Boc-Asp-MeThr-OR | Boc-Asp-MeCys-OR | Boc-Asp-MeMet-OR |
| Boc-Glu | Boc-Glu-MeSer-OR | Boc-Glu-MeThr-OR | Boc-Glu-MeCys-OR | Boc-Glu-MeMet-OR |
| Boc-Asn | Boc-Asn-MeSer-OR | Boc-Asn-MeThr-OR | Boc-Asn-MeCys-OR | Boc-Asn-MeMet-OR |
| Boc-Gln | Boc-Gln-MeSer-OR | Boc-Gln-MeThr-OR | Boc-Gln-MeCys-OR | Boc-Gln-MeMet-OR |
| Boc-Lys | Boc-Lys-MeSer-OR | Boc-Lys-MeThr-OR | Boc-Lys-MeCys-OR | Boc-Lys-MeMet-OR |
| Boc-Trp | Boc-Trp-MeSer-OR | Boc-Trp-MeThr-OR | Boc-Trp-MeCys-OR | Boc-Trp-MeMet-OR |
| Boc-His | Boc-His-MeSer-OR | Boc-His-MeThr-OR | Boc-His-MeCys-OR | Boc-His-MeMet-OR |
| Boc-Arg | Boc-Arg-MeSer-OR | Boc-Arg-MeThr-OR | Boc-Arg-MeCys-OR | Boc-Arg-MeMet-OR |
| Boc-Pro | Boc-Pro-MeSer-OR | Boc-Pro-MeThr-OR | Boc-Pro-MeCys-OR | Boc-Pro-MeMet-OR |
| Boc-Orn | Boc-Orn-MeSer-OR | Boc-Orn-MeThr-OR | Boc-Orn-MeCys-OR | Boc-Orn-MeMet-OR |

| | MeAsp-OR | MeGlu-OR | MeAsn-OR |
|---|---|---|---|
| Boc-Gly | Boc-Gly-MeAsp-OR | Boc-Gly-MeGlu-OR | Boc-Gly-MeAsn-OR |
| Boc-Ala | Boc-Ala-MeAsp-OR | Boc-Ala-MeGlu-OR | Boc-Ala-MeAsn-OR |
| Boc-Val | Boc-Val-MeAsp-OR | Boc-Val-MeGlu-OR | Boc-Val-MeAsn-OR |
| Boc-Leu | Boc-Leu-MeAsp-OR | Boc-Leu-MeGlu-OR | Boc-Leu-MeAsn-OR |
| Boc-Ile | Boc-Ile-MeAsp-OR | Boc-Ile-MeGlu-OR | Boc-Ile-MeAsn-OR |
| Boc-Phe | Boc-Phe-MeAsp-OR | Boc-Phe-MeGlu-OR | Boc-Phe-MeAsn-OR |
| Boc-Tyr | Boc-Tyr-MeAsp-OR | Boc-Tyr-MeGlu-OR | Boc-Tyr-MeAsn-OR |
| Boc-Ser | Boc-Ser-MeAsp-OR | Boc-Ser-MeGlu-OR | Boc-Ser-MeAsn-OR |
| Boc-Thr | Boc-Thr-MeAsp-OR | Boc-Thr-MeGlu-OR | Boc-Thr-MeAsn-OR |
| Boc-Cys | Boc-Cys-MeAsp-OR | Boc-Cys-MeGlu-OR | Boc-Cys-MeAsn-OR |
| Boc-Met | Boc-Met-MeAsp-OR | Boc-Met-MeGlu-OR | Boc-Met-MeAsn-OR |
| Boc-Asp | Boc-Asp-MeAsp-OR | Boc-Asp-MeGlu-OR | Boc-Asp-MeAsn-OR |
| Boc-Glu | Boc-Glu-MeAsp-OR | Boc-Glu-MeGlu-OR | Boc-Glu-MeAsn-OR |
| Boc-Asn | Boc-Asn-MeAsp-OR | Boc-Asn-MeGlu-OR | Boc-Asn-MeAsn-OR |
| Boc-Gln | Boc-Gln-MeAsp-OR | Boc-Gln-MeGlu-OR | Boc-Gln-MeAsn-OR |
| Boc-Lys | Boc-Lys-MeAsp-OR | Boc-Lys-MeGlu-OR | Boc-Lys-MeAsn-OR |
| Boc-Trp | Boc-Trp-MeAsp-OR | Boc-Trp-MeGlu-OR | Boc-Trp-MeAsn-OR |
| Boc-His | Boc-His-MeAsp-OR | Boc-His-MeGlu-OR | Boc-His-MeAsn-OR |
| Boc-Arg | Boc-Arg-MeAsp-OR | Boc-Arg-MeGlu-OR | Boc-Arg-MeAsn-OR |
| Boc-Pro | Boc-Pro-MeAsp-OR | Boc-Pro-MeGlu-OR | Boc-Pro-MeAsn-OR |
| Boc-Orn | Boc-Orn-MeAsp-OR | Boc-Orn-MeGlu-OR | Boc-Orn-MeAsn-OR |

| | MeGln-OR | MeLys-OR | MeTrp-OR |
|---|---|---|---|
| Boc-Gly | Boc-Gly-MeGln-OR | Boc-Gly-MeLys-OR | Boc-Gly-MeTrp-OR |
| Boc-Ala | Boc-Ala-MeGln-OR | Boc-Ala-MeLys-OR | Boc-Ala-MeTrp-OR |
| Boc-Val | Boc-Val-MeGln-OR | Boc-Val-MeLys-OR | Boc-Val-MeTrp-OR |
| Boc-Leu | Boc-Leu-MeGln-OR | Boc-Leu-MeLys-OR | Boc-Leu-MeTrp-OR |
| Boc-Ile | Boc-Ile-MeGln-OR | Boc-Ile-MeLys-OR | Boc-Ile-MeTrp-OR |
| Boc-Phe | Boc-Phe-MeGln-OR | Boc-Phe-MeLys-OR | Boc-Phe-MeTrp-OR |
| Boc-Tyr | Boc-Tyr-MeGln-OR | Boc-Tyr-MeLys-OR | Boc-Tyr-MeTrp-OR |
| Boc-Ser | Boc-Ser-MeGln-OR | Boc-Ser-MeLys-OR | Boc-Ser-MeTrp-OR |
| Boc-Thr | Boc-Thr-MeGln-OR | Boc-Thr-MeLys-OR | Boc-Thr-MeTrp-OR |
| Boc-Cys | Boc-Cys-MeGln-OR | Boc-Cys-MeLys-OR | Boc-Cys-MeTrp-OR |
| Boc-Met | Boc-Met-MeGln-OR | Boc-Met-MeLys-OR | Boc-Met-MeTrp-OR |
| Boc-Asp | Boc-Asp-MeGln-OR | Boc-Asp-MeLys-OR | Boc-Asp-MeTrp-OR |
| Boc-Glu | Boc-Glu-MeGln-OR | Boc-Glu-MeLys-OR | Boc-Glu-MeTrp-OR |
| Boc-Asn | Boc-Asn-MeGln-OR | Boc-Asn-MeLys-OR | Boc-Asn-MeTrp-OR |
| Boc-Gln | Boc-Gln-MeGln-OR | Boc-Gln-MeLys-OR | Boc-Gln-MeTrp-OR |
| Boc-Lys | Boc-Lys-MeGln-OR | Boc-Lys-MeLys-OR | Boc-Lys-MeTrp-OR |
| Boc-Trp | Boc-Trp-MeGln-OR | Boc-Trp-MeLys-OR | Boc-Trp-MeTrp-OR |
| Boc-His | Boc-His-MeGln-OR | Boc-His-MeLys-OR | Boc-His-MeTrp-OR |
| Boc-Arg | Boc-Arg-MeGln-OR | Boc-Arg-MeLys-OR | Boc-Arg-MeTrp-OR |
| Boc-Pro | Boc-Pro-MeGln-OR | Boc-Pro-MeLys-OR | Boc-Pro-MeTrp-OR |
| Boc-Orn | Boc-Orn-MeGln-OR | Boc-Orn-MeLys-OR | Boc-Orn-MeTrp-OR |

| | MeHis-OR | MeArg-OR | MeOrn-OR |
|---|---|---|---|
| Boc-Gly | Boc-Gly-MeHis-OR | Boc-Gly-MeArg-OR | Boc-Gly-MeOrn-OR |
| Boc-Ala | Boc-Ala-MeHis-OR | Boc-Ala-MeArg-OR | Boc-Ala-MeOrn-OR |
| Boc-Val | Boc-Val-MeHis-OR | Boc-Val-MeArg-OR | Boc-Val-MeOrn-OR |
| Boc-Leu | Boc-Leu-MeHis-OR | Boc-Leu-MeArg-OR | Boc-Leu-MeOrn-OR |
| Boc-Ile | Boc-Ile-MeHis-OR | Boc-Ile-MeArg-OR | Boc-Ile-MeOrn-OR |
| Boc-Phe | Boc-Phe-MeHis-OR | Boc-Phe-MeArg-OR | Boc-Phe-MeOrn-OR |
| Boc-Tyr | Boc-Tyr-MeHis-OR | Boc-Tyr-MeArg-OR | Boc-Tyr-MeOrn-OR |
| Boc-Ser | Boc-Ser-MeHis-OR | Boc-Ser-MeArg-OR | Boc-Ser-MeOrn-OR |
| Boc-Thr | Boc-Thr-MeHis-OR | Boc-Thr-MeArg-OR | Boc-Thr-MeOrn-OR |
| Boc-Cys | Boc-Cys-MeHis-OR | Boc-Cys-MeArg-OR | Boc-Cys-MeOrn-OR |
| Boc-Met | Boc-Met-MeHis-OR | Boc-Met-MeArg-OR | Boc-Met-MeOrn-OR |
| Boc-Asp | Boc-Asp-MeHis-OR | Boc-Asp-MeArg-OR | Boc-Asp-MeOrn-OR |
| Boc-Glu | Boc-Glu-MeHis-OR | Boc-Glu-MeArg-OR | Boc-Glu-MeOrn-OR |
| Boc-Asn | Boc-Asn-MeHis-OR | Boc-Asn-MeArg-OR | Boc-Asn-MeOrn-OR |
| Boc-Gln | Boc-Gln-MeHis-OR | Boc-Gln-MeArg-OR | Boc-Gln-MeOrn-OR |
| Boc-Lys | Boc-Lys-MeHis-OR | Boc-Lys-MeArg-OR | Boc-Lys-MeOrn-OR |
| Boc-Trp | Boc-Trp-MeHis-OR | Boc-Trp-MeArg-OR | Boc-Trp-MeOrn-OR |
| Boc-His | Boc-His-MeHis-OR | Boc-His-MeArg-OR | Boc-His-MeOrn-OR |
| Boc-Arg | Boc-Arg-MeHis-OR | Boc-Arg-MeArg-OR | Boc-Arg-MeOrn-OR |
| Boc-Pro | Boc-Pro-MeHis-OR | Boc-Pro-MeArg-OR | Boc-Pro-MeOrn-OR |
| Boc-Orn | Boc-Orn-MeHis-OR | Boc-Orn-MeArg-OR | Boc-Orn-MeOrn-OR |

The term “amino acid side chain” is used herein in its broadest sense and refers to the side chains of both L- and D-amino acids including the 20 common amino acids such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine; the less common amino acids but known derivatives such as cystine, 5-hydroxylysine, 4-hydroxyproline, α-aminoadipic acid, α-amino-n-butyric acid, 3,4-dihydroxyphenylalanine, homoserine, α-methylserine, ornithine, pipecolic acid and thyroxine; and any amino acid having a molecular weight less than about 500.

### EXAMPLES

The invention will now be described with reference to the following examples. These examples are not to be construed as limiting the invention in any way.

All melting points are uncorrected and were recorded on a microscope hot-stage apparatus. Infrared spectra were recorded on a FTIR spectrometer, using a diffuse reflectance accessory with KBr background. Standard pulse sequences (HMBC, HSQC, COSY, and DEPT) were used to identify compound 66. Electrospray mass spectra (E.S.M.S.) were obtained on a triple quadrupole mass spectrometer using water/methanol/acetic acid (0:99:1 or 50:50:1) mixtures as the mobile phase. Low resolution mass spectra (e.i.) were performed. Other low and high resolution mass spectra (l.s.i.m.s.) were measured. Ethyl acetate and hexane used for chromatography were distilled prior to use. All solvents were purified by distillation. For dry solvents, procedures from Perrin and Armarego²⁹ were followed. Dry dichloromethane was distilled and stored over Linde type 4 Å molecular sieves. All other reagents and solvents were purified or dried as described by Perrin and Armarego.²⁹

### Example 1

### Serine, Threonine and Tyrosine

The formation of the 5-oxazolidinones of serine and threonine is complicated by participation of the sidechain hydroxyls to form oxazolidines as shown in structures (5) and (6) and, in the case of threonine, this intermediate was also produced in the attempted reductive cleavage²⁷. Thus, for sidechain protection, several strategies were considered.

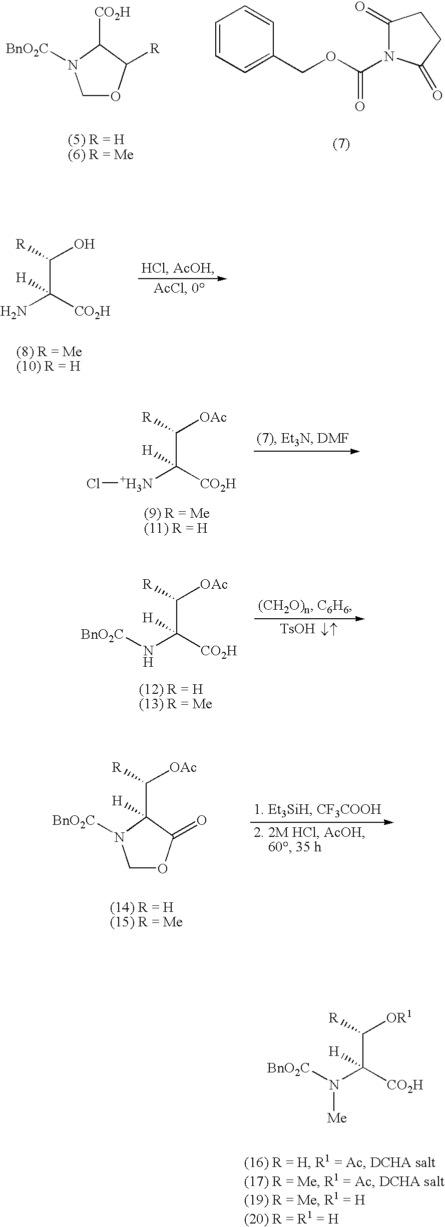

While a number of protected derivatives, of serine and threonine are known ³⁰⁻³³ and were considered, in the end, the simple expedient of acetylation fulfilled all the objectives. L-Threonine (8) was used to prepare O-acetyl threonine (9) in high yield according to the method of Wilchek and Patchornik (Scheme 10).³⁴ This procedure was equally successful with L-serine (10) in providing the acetate (11).

The formation of the 5-oxazolidinones (14) (87%) and (15) (91%) using the intermediates (12) and (13), respectively proceeded in high yield. Reductive cleavage gave the N-methyl-O-acetyl amino acids (16) (74%) and (17) (80%) as their dicyclohexylamine salts. These acetates are in suitable form for use in solution and solid phase coupling procedures but the deprotection procedure required additional examination. Hydrolysis of the acetate esters under basic conditions has been reported in relation to serine derivatives³⁵ but was unsuccessful in this study with the threonine acetate (17a). Attempted base hydrolyses of the threonine acetate (17a) always resulted in isolation of the starting material and this was attributed to the in situ formation of the tetrahedral intermediate (18) (FIG. 2) which survived the hydrolytic conditions and returned the starting material upon acidic work-up.

[image]

Conversely, aqueous acidic conditions and mild heating (Scheme 10) removed the acetate in high yield to give the alcohol (19) (88%). The same sequence of reactions works well for the serine intermediates (14), (16) and (20). Confirmation that this synthetic sequence did not reduce the optical purity was obtained by hydrogenolysis of the threonine carbamate (19) (Scheme 11). The isolated N-methyl-L-Threonine (21) had an optical rotation of [α]_{D} −14° (c=1, 6M HCl) which matched previously reported values.²⁸ Thus, N-methyl serine and threonine with and without sidechain protection, are available by a side chain O-acetyl protection strategy.

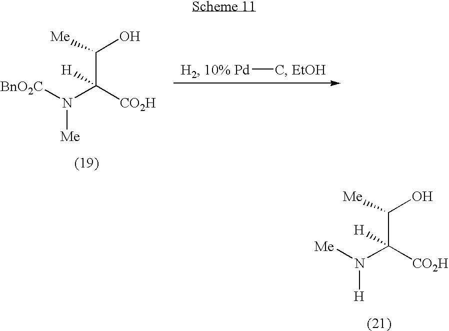

Tyrosine forms the expected oxazolidinone without sidechain protection but the yields for its formation (37%) and subsequent reductive cleavage (60%) were lower than desired. Given the success of sidechain acetylation in the serine and threonine manipulations, a similar strategy was attempted with tyrosine. Solubility problems were encountered with the Fmoc carbamate of tyrosine and its conversion to the corresponding acetate. The commercially available tyrosine benzyl ether (22) suited the oxazolidinone chemistry, and the oxazolidinone (23) was isolated in 86% yield (Scheme 12). Reductive cleavage then gave the N-methyl tyrosine (24) in 70% yield: a substantial improvement compared with the previous sequence in which the hydroxy group was unprotected.

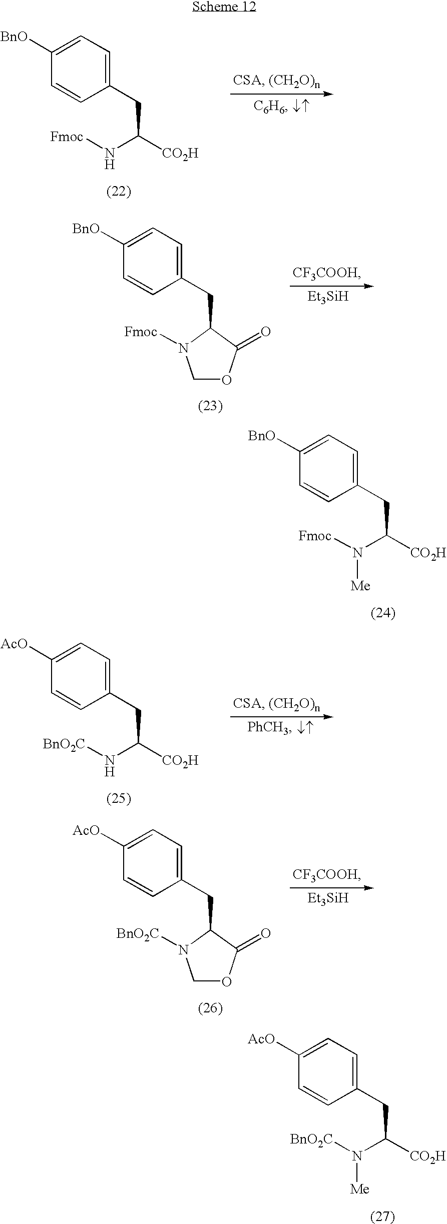

The tyrosine benzyl carbamate (25)³⁶ was also converted to the oxazolidinone (26) (89%) and reductive cleavage afforded the N-methyl tyrosine O-acetate (27) (88%). Formation of the N-methyl tyrosine (27) represents a 40% improvement compared to the tyrosine sequence in which the hydroxy group was unprotected.

### (S)-3-Benzyloxycarbonyl-4-acetoxymethyloxazolidin-5-one (14)

To a sample of the carbamate (12) (1.11 g, 4.0 mmol) in toluene (50 ml) was added camphorsulfonic acid (70 mg) and dry paraformaldehyde (1.0 g). The reaction mixture was then heated to reflux for 30-60 mins [monitored by TLC (40% ethyl acetate-hexane)]. The mixture was cooled, filtered to remove solids and diluted with ether (150 ml). The ethereal solution was washed with 2.5% aqueous sodium bicarbonate solution (4×30 ml).

The combined aqueous layers were extracted with ether (30 ml) and the combined ethereal layers were dried (MgSO₄), filtered and concentrated in vacuo to give the oxazolidinone (14) as an oil (1.01 g, 87%). A small sample was further purified by flash chromatography on silica eluting with 30% ethyl acetate-hexane. [α]_{D}²⁴ +110.7° (c 1.0, CHCl₃). ¹H NMR (300 MHz, CDCl₃) 7.33 (s, 5H), 5.51 (bs, 1H), 5.20 (d, 1H, J=3.8 Hz), 5.16 (s, 2H), 4.61-4.58 (m, 1H) 4.42-4.32 (m, 2H), 1.99 (s, 3H). ¹³C NMR (75 MHz, CDCl₃) 169.85, 152.30, 135.07, 128.57, 128.25, 78.39, 68.04, 62.19, 54.41, 20.44. IR (NaCl) ν 3090, 3065, 3034 and 3010 (CH, aromatic), 3000-2900 (CH, saturated), 1807 (C═O, oxazolidinone), 1746 (C═O, acetate), 1719 (C═O, carbamate), 1500, 1452, 1419, 1359, 1315, 1290, 1234, 1170, 1130, 1060, 1034, 969, 945, 765, 699 cm⁻¹. Anal. Calcd for C₁₄H₁₅NO₆: C, 57.34; H, 5.16; N, 4.78. Found: C, 57.54; H, 5.26; N, 4.96.

### (4S)-3-Benzyloxycarbonyl-4-[(1S)-acetoxyethyl]oxazolidin-5-one (15)

To a sample of the carbamate (13) (1.47 g, 5 mmol) in toluene (50 ml) was added camphorsulfonic acid (70 mg) and dry paraformaldehyde (1.0 g). The reaction mixture was then heated to reflux for 30-60 mins [monitored by TLC (40% ethyl acetate-hexane)). The mixture was cooled, filtered to remove solids and diluted with ether (150 ml). The ethereal solution was washed with 2.5% aqueous sodium bicarbonate solution (4×30 ml). The combined aqueous layers were extracted with ether (30 ml) and the combined ethereal layers were dried (MgSO₄), filtered and concentrated in vacuo to give the oxazolidinone (15) as an oil (1.38 g, 91%). A small sample was further purified by flash chromatography on silica eluting with 30% ethyl acetate-hexane. [α]_{D}²⁴ +120.7° (c 2.0, CHCl₃). ¹H NMR (300 MHz, CDCl₃) 7.32 (s, 5H), 5.70 (bs, 1H), 5.29-5.18 (m, 4H), 4.41 (bs, 1H), 1.97 (s, 3H), 1.34-1.31 (m, 3H). ¹³C NMR (75 MHz, CDCl₃) δ_{C}170.14, 169.19, 153.83, 134.98, 128.59, 128.37, 78.98, 70.59, 68.34, 59.11, 20.76, 16.63. IR (NaCl) ν 3092, 3066 and 3033 (CH, aromatic), 3000-2900 (CH, saturated), 1808 (C═O, oxazolidinone), 1742 (C═O, acetate), 1719 (C═O, carbamate), 1498, 1454, 1409, 1360, 1328, 1232, 1169, 1124, 1041, 955, 897, 753, 700 cm⁻¹ MS (l.s.i.m.s.) m/z 308 (M+1, 90%), 289, (50), 264 (100). HRMS calcd for C₁₅H₁₇NO₆ (M+1) 308.1134 found 308.1142. Anal. Calcd for C₁₅H₁₇NO₆: C, 58.63; H, 5.58; N, 4.56. Found: C, 58.62; H, 5.63; N, 4.71.

### N-Benzyloxycarbonyl-N-methyl-L-serine-O-acetate (16)

A sample of the oxazolidinone (14) (1.18 g, 4.0 mmol) was dissolved in chloroform (20 ml) at room temperature and triethylsilane (1.89 ml) was added followed by trifluoroacetic acid (20 ml) and the reaction mixture was left to stand for 3-4 d. The reaction mixture was concentrated under reduced pressure. To the residue was added toluene (50 ml) and the mixture was again concentrated in vacuo. This procedure was repeated with more toluene (50 ml). The residue was then diluted with ether and extracted with saturated aqueous sodium bicarbonate solution (4×30 ml). The combined aqueous extracts were washed with ether and then acidified to pH 2 with 5 M hydrochloric acid. The aqueous phase was then extracted with ether (3×50 ml). The combined ethereal extracts were dried (MgSO₄), filtered and evaporated under reduced pressure to approximately 20 ml volume. Dicyclohexylamine (DCHA) (0.8 ml) was added and any solid, which formed immediately, was filtered off. The clear filtrate was left to stand overnight during which the N-methyl serine acetate (16) precipitated as its DCHA salt. (1.40 g, 74%). Mp 135-147° C. [α]_{D}²⁵ −8° (c 2.0, CHCl₃). ¹H NMR (300 MHz, CDCl₃) 9.15 (bs, 2H), 7.33-7.24 (m, 5H), 5.21-4.99 (m, 2H), 4.84 (td, 1H, J═10.0, 4.2 Hz), 4.55 (dt, 1H, J═11.8, 3.8 Hz), 4.42-4.26 (m, 1H), 2.97-2.88 (m, 5H), 1.97-1.13 (m, 23H). ¹³C NMR (75 MHz, CDCl₃) (rotamers) δ6172.30, 172.14, 170.85, 156.97, 156.77, 137.01, 128.37, 127.81, 127.58, 127.52, 66.97, 66.89, 62.54, 62.48, 59.99, 59.75, 52.75, 31.14, 28.95, 25.04, 24.67, 20.83, 20.74. IR (KBr disk)ν 3062, 3034 and 3005 (CH, aromatic), 3000-2800 (CH, saturated), 2476 and 2417 (NH₂⁺), 1738 (C═O, acetate), 1693 (C═O, carbamate), 1641 (CO₂⁻), 1566, 1441, 1392, 1370, 1345, 1312, 1286, 1250, 1149, 1075, 696 cm⁻¹. Anal. Calcd for C₂₆H₄₀N₂O₆: C, 65.52; H, 8.46; N, 5.88. Found: C, 65.52; H, 8.65; N, 5.86.

### N-Benzyloxycarbonyl-N-methyl-L-threonine-O-acetate (17)

A sample of the oxazolidinone (15) (1.22 g, 4.0 mmol) was dissolved in chloroform (20 ml) at room temperature and triethylsilane (1.89 ml) was added followed by trifluoroacetic acid (20 ml) and the reaction mixture was left to stand for 3-4 d. The reaction mixture was concentrated under reduced pressure. To the residue was added toluene (50 ml) and the mixture was again concentrated in vacuo. This procedure was repeated with more toluene (50 ml). The residue was then diluted with ether and extracted with saturated aqueous sodium bicarbonate solution (4×30 ml). The combined aqueous extracts were washed with ether and then acidified to pH 2 with 5 M hydrochloric acid. The aqueous phase was then extracted with ether (3×50 ml). The combined ethereal extracts were dried (MgSO₄), filtered and evaporated under reduced pressure to approximately 20 ml volume. Dicyclohexylamine (DCHA) (0.8 ml) was added and any solid, which formed immediately, was filtered off. The filtrate solution was left to stand overnight during which the N-methyl threonine acetate (17) precipitated as its DCHA salt (1.57 g, 80%).

### N-Benzyloxycarbonyl-N-methyl-L-serine (20)

A sample of the serine DCHA salt (16) (970 mg, 2.0 mmol) was suspended in a mixture of dioxane and 2M hydrochloric acid (20 ml, 1:1) with stirring. The mixture was then heated to 60° C. for ca. 30 h (TLC). The reaction mixture was then diluted with water (300 ml) and extracted with ether (3×100 ml). The combined organic phases were dried (MgSO₄), filtered and evaporated at reduced pressure to give the N-methyl serine (19) as an oil (480 mg, 95%), which was identical in all respects with previously reported material.²⁸

### N-Benzyloxycarbonyl-N-methyl-L-threonine (19)

A sample of the threonine DCHA salt (17) (1.04 g, 2.0 mmol) was suspended in a mixture of dioxane and 2M hydrochloric acid (20 ml, 1:1) with stirring. The mixture was then heated to 60° C. for ca. 30 h (TLC). The reaction mixture was then diluted with water (300 ml) and extracted with ether (3×100 ml). The combined organic phases were dried (MgSO₄), filtered and evaporated at reduced pressure to give the N-methyl threonine (19) as an oil (520 mg, 97%), which was identical in all respects with previously reported material.²⁸

### N-Methyl-L-threonine (21)

A small sample of the carbamate (19) was hydrogenolysed over 10% palladium on charcoal catalyst.²⁸ The material isolated had [α]_{D}²⁵ −14° (c, 0.5 in 6 M HCl) which was identical with authentic material.²⁸

### (S)-3-(Carbonyl-9H-fluoren-9-ylmethoxy)-4-(4-benzyloxybenzyl)-oxazolidin-5-one (23)

To a sample of the carbamate (22) (470 mg, 0.9 mmol) in toluene (150 ml) was added camphorsulfonic acid (66 mg). The reaction mixture was then heated to reflux for 4 h during which dry paraformaldehyde (500 mg) was added in small portions down the condenser. The mixture was then cooled, filtered to remove solids and the filtrate was evaporated under reduced pressure. The residue was taken up in ethyl acetate and washed with saturated aqueous sodium bicarbonate solution (3×30 ml). The organic layer was dried (MgSO₄), filtered and concentrated in vacuo. The residue was purified by flash chromatography on silica eluting with 25% ethyl acetate-hexane to give the oxazolidinone (23) as a foam (405 mg, 86%). [α]_{D}²² +132.5° (c 1.0, Et₂O). ¹H NMR (300 MHz, CDCl₃) (rotamers) 7.77-6.60 (m, 17H), 5.11 (brs, 1H), 4.99-4.95 (m, 1H), 4.73-4.64 (m, 1H), 4.47 (m, 0.5H), 4.27-4.19 (m, 1H), 4.10 (m, 1H), 3.94 (m, 0.5H), 3.32-2.35 (m, 2H). ¹³C NMR (75 MHz, CDCl₃) (rotamers) δ171.54, 157.84, 151.84, 143.09, 141.12, 136.50, 126.19, 130.31, 128.25, 127.67, 127.17, 126.93, 124.20, 119.87, 119.80, 114.76, 77.49, 69.56, 67.10, 66.31, 56.05, 46.93, 34.20. IR (KBr disk) ν 3034 (CH, aromatic), 3000-2800 (CH, saturated), 1800 (C═O, oxazolidinone), 1717 (C═O, carbamate), 1610, 1511, 1451, 1422, 1357, 1300, 1242, 1177, 1159, 1129, 1052, 1024, 830, 759, 741, 696 cm⁻¹. HRMS calcd for C₃₂H₂₇NO₅ (M⁺) 505.1968, found 505.1891.

### (S)-3-Benzyloxycarbonyl-4-(4-acetoxybenzyl)-oxazolidin-5-one (26)

To a sample of the carbamate (25) (2.90 g, 7.9 mmol) in toluene (50 ml) was added camphorsulfonic acid (200 mg). To the reaction mixture was added dry paraformaldehyde (3.0 g) and the mixture was heated to reflux for 1 h. The mixture was then cooled, filtered to remove solids and the filtrate was evaporated under reduced pressure. The residue was taken up in ether (100 ml). The ether layer was washed with 5% sodium carbonate solution (3×50 ml) followed by water and then brine. The 30 organic layer was dried (MgSO₄), filtered and concentrated in vacuo. The residue (2.80 g) was purified by flash chromatography on silica eluting with 20% ethyl acetate-hexane to give the oxazolidinone (26) as a clear colourless oil (2.61 g, 89%). [α]_{D}²⁴ +172.3° (c 1.0, CHCl₃). ¹H NMR (300 MHz, CDCl₃) 7.35-7.29 and 7.21-6.91 (2m, 9H), 5.28-5.14 (m, 3H), 4.49 (brs, 1H), 4.32 (d, 1H, J=3.9 Hz), 3.42-3.08 (m, 2H), 2.23 (s, 3H). ¹³C NMR (75 MHz, CDCl₃) δ 171.51, 168.95, 152.06, 149.93, 135.37, 131.91, 130.42, 128.51, 128.25, 121.70, 77.78, 67.62, 56.11, 35.33, 34.31, 20.85. IR (NaCl)ν 3100, 3062 and 3034 (CH, aromatic), 3000-2800 (CH, saturated), 1800 (C═O, oxazolidinone), 1760 (C═O, acetate), 1716 (C═O, carbamate), 1604, 1506, 1416, 1361, 1310, 1202, 1126, 1049, 1013, 912, 843, 754 cm⁻¹. Anal. Calcd for C₂₀H₁₉NO₆: C, 65.03; H, 5.18; N, 3.79. Found: C, 64.89; H, 5.35; N, 3.87.

### N-(Carbonyl-9H-fluoren-9-ylmethoxy)-N-methyl-L-tyrosine-O-benzyl ether (24)

A sample of the oxazolidinone (23) (95 mg, 0.2 mmol) was dissolved in dichloromethane (5 ml) at room temperature and triethylsilane (270 μl, 1.7 mmol) was added followed by trifluoroacetic acid (1.2 ml, 10.5 mmol) and the reaction mixture was left to stand for 2 d. The reaction mixture was concentrated under reduced pressure. To the residue was added dichloromethane (5 ml) and the mixture was again concentrated in vacuo. This procedure was repeated with toluene (5 ml) until traces of trifluoroacetic acid were removed. The residue was then diluted with ethyl acetate and extracted with saturated aqueous sodium bicarbonate solution (3×30 ml). The combined aqueous extracts were washed with ether and then acidified to pH 2 with 2 M hydrochloric acid. The aqueous phase was then extracted with ethyl acetate (3×50 ml). The combined organic extracts were dried (MgSO₄), filtered and evaporated under reduced pressure. The residue was purified by flash chromatography eluting with 95:4:1 dichloromethane/methanol/acetic acid to yield the N-methyl acid (24) as a white foam (67 mg, 70%). [α]_{D}²⁴ −1.6° (c 0.5, Et₂O). R_{f} 0.3 (95:4:1 dichloromethane/methanol/acetic acid). ¹H NMR (300 MHz, CDCl₃) (rotamers) 7.74-6.46 (m, 17H), 4.90-4.07 (m, 6H), 3.27 and 3.08-2.96 and 2.66-2.63 (dd and 2m, 1H and 2H, J=4.8, 14.4 Hz), 2.78 and 2.74 (2s, 3H). ¹C NMR (75 MHz, CDCl₃) (rotamers) δ 174.21, 156.70, 155.85, 154.28, 143.41, 143.31, 140.93, 129.57, 128.24, 127.55, 126.73, 124.64, 124.31, 119.62, 115.20, 67.62, 67.10, 60.59, 60.26, 46.81, 46.69, 33.61, 33.46, 31.92.

### N-Benzyloxycarbonyl-N-methyl-L-tyrosine-O-acetate (27)

A sample of the oxazolidinone (26) (1.50 g, 4.1 mmol) was dissolved in chloroform (20 ml) at room temperature and triethylsilane (1.9 ml) was added followed by trifluoroacetic acid (20 ml) and the reaction mixture was left to stand for 4 d. The reaction mixture was diluted with toluene and concentrated under reduced pressure. This procedure was repeated with a further aliquot of toluene (50 ml). The residue was then diluted with ether and extracted with 5% sodium carbonate solution (4×20 ml). The combined aqueous extracts were washed with ether and then acidified to pH 2 with 5 M hydrochloric acid. The aqueous phase was then extracted with dichloromethane (3×50 ml). The combined extracts were dried (MgSO₄), filtered and evaporated under reduced pressure to afford a clear oil (1.33 g, 88%). A sample of the oil was converted to the t-butylamine salt by dissolution in ether and addition of t-butylamine (1.1 equiv.) followed by hexane until the solution turned slightly cloudy. The turbid solution was then left to stand at room temperature for 4 h and then at 0° C. overnight during which the N-methyl tyrosine acetate (27) precipitated as its t-butylammonium salt. Mp 54-60° C. [α]_{D}²⁴ −34.3° (c 1.0, CHCl₃). ¹H NMR (300 MHz, CDCl₃) (rotamers) 7.74 (brs, 3H), 7.29-6.88 (m, 9H), 5.01-4.64 (m, 3H), 3.38-3.28 and 3.00-2.77 (2m, 5H), 2.26 (s, 3H), 1.23 (s, 9H). ¹³C NMR (75 MHz, CDCl₃) (rotamers) δ175.95, 169.51, 156.89, 156.50, 148.99, 136.78, 136.66, 136.60, 136.50, 129.68, 128.39, 127.76, 127.45, 121.32, 66.97, 66.86, 62.64, 62.34, 51.23, 35.51, 35.05, 31.90, 31.08, 27.51, 21.10. IR (KBr disk). ν3121, 3064 and 3033 (CH, aromatic), 3000-2800 (CH, saturated), 2622 and 2529, ⁺NH₃, 1760 (C═O, acetate), 1674 (C═O, carbamate), 1592 (CO₂⁻), 1507, 1448, 1377, 1314, 1209, 1194, 1134, 750, 695, 639 cm⁻¹. Anal. Calcd for C₂₄H₃₂N₂O₆: C, 64.85; H, 7.26; N, 6.30. Found: C, 64.71; H, 7.39; N, 6.41.

### Example 2

### Cysteine and Cystine

The synthesis of the sulfur bearing N-methyl amino acids gave mixed results using the 5-oxazolidinone route (FIG. 3).²⁸ The cysteine carbamate (28a) gave the oxazolidinone (29a) in only 3% yield. Methionine, on the other hand, gave the oxazolidinone (29b) in 91%. The reductive cleavage of the oxazolidinone (29a) gave the thiazolidine (30) exclusively indicating the requisite iminium ion had been formed and was then intercepted intramolecularly by the thiol. The methionine intermediate (29b) gave a mixture of products.

[image]

To lessen the nucleophilicity of the thiol, the S-acetyl cysteine derivative (31a)^{37,38} was prepared and this underwent oxazolidination in moderate yield (51%) (Scheme 13). However, attempted reductive cleavage of the oxazolidinone (32) gave no N-methyl products upon workup. Thus the S-benzyl cysteine (31c)³⁹ was converted to the oxazolidinone (33) in high yield (89%) and subsequent reductive cleavage with trifluoroacetic acid and triethylsilane gave the expected N-methyl amino acid (34) (70%). Removal of the S-benzyl group in any subsequent sequence may present problems given the preferred method for debenzylation involves treatment with HF.³⁸ Thus protection using a (S-PMB (p-methoxybenzyl) acid (31d) was proposed, as the ultimate removal of the PMB ether can be effected with refluxing trifluoroacetic acid.⁴⁰ The ether was as prepared but attempts to convert it to the corresponding oxazolidinone resulted in decomposition.

However, the formation of N-methyl cysteine is performed efficiently by the related method of Yamashiro et al³⁹ which involves the reaction of cysteine with paraformaldehyde to give a thiazolidine carboxylic acid. A dissolving metal reductive cleavage of the thiazolidine ring generates N-methyl cysteine, which can then be converted in many ways to a range of synthetically useful intermediates including the S-benzyl carbamate (34).

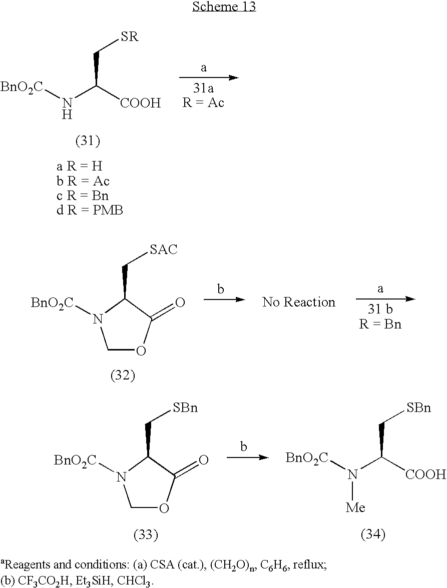

During the studies to solve the cysteine manipulation problems, the use of the cystine carbamate (35) (Scheme 14) was also trialed. Oxazolidinone formation gave the dimeric structure (36) as a solid (33%). However, the reductive cleavage resulted in isolation of the thiazolidine (30). Evidently, the disulfide bridge is cleaved initially giving the cysteine oxazolidinone (31a) in situ. This was then transformed into the expected iminium ion, which reacts with the thiol, as before, to give the thiazolidine (30).

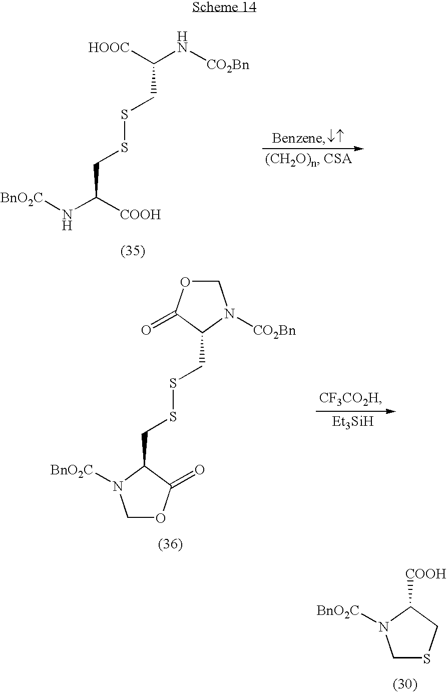

It was previously reported²⁷ that oxazolidination of cysteine led to the formation of the dimeric structure (37). In reality this dimeric structure is a proton sharing aggregate of two thiazolidines (30) that forms in the ESMS. The proposal of the structure (37) was based on the observance in the electrospray mass spectrum of m/z 535. However, further analysis of the cysteine product revealed the appearance of the m/z 535 peak was concentration dependent. Furthermore, while the ESMS. of the putative aggregate also exhibited peaks at m/z 557 and 268 corresponding to M+Na and M+2/2, that same spectrum did not show a peak at m/z 279 for M+Na+H/2. The m/z 268 peak is revealed as the M+H ion for the thiazolidine (30). Thus, the dimer (37) is not formed in the cysteine oxazolidination; only the thiazolidine (30) is formed in that reaction.

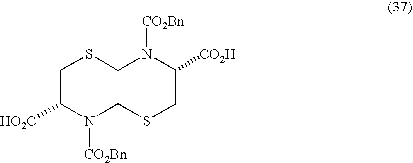

### (R)-3-Benzyloxycarbonyl-4-(acetylthiomethyl)oxazolidin-5-one (32)

In a round-bottomed flask fitted with a Dean-Stark apparatus, a mixture of the S-acetyl cysteine (31b) (1.0 g, 3.4 mmol), paraformaldehyde (450 mg) and camphorsulfonic acid (40 mg) was suspended in benzene (30 ml). The mixture was heated to reflux for 3 h (monitored by TLC). The reaction mixture was then concentrated at reduced pressure. The residue was taken up in ethyl acetate and the organic layer was washed with saturated aqueous sodium bicarbonate solution to remove acidic material. The organic layer was dried (MgSO₄), filtered and evaporated in vacuo. The residue was purified by column chromatography, eluting with 50% ethyl acetate-hexane to afford the oxazolidinone (32) as an oil (540 mg, 51%). [α]_{D}²⁵ +101.0° (c 0.9, CHCl₃). ¹H NMR (300 MHz, CDCl₃) 7.35-7.30 (m, 5H), 5.44 (bs, 1H), 5.22-5.14 (m, 3H), 4.52 (bs, 1H), 3.65 (dd, 1H, J=4.7, 14.2 Hz), 3.41-3.30 (m, 1H), 2.29 (s, 3H). ¹³C NMR (75 MHz, CDCl₃) δ 193.03, 170.28, 152.39, 135.17, 128.51, 128.45, 128.24, 127.82, 78.39, 68.03, 54.60, 30.36, 29.36. IR (NaCl) ν 3110, 3090, 3065 and 3034 (CH, aromatic), 3000-2800 (CH, saturated), 1804 (C═O, oxazolidinone), 1714 (C═O, carbamate, acetate), 1500, 1412, 1357, 1290, 1215, 1168, 1129, 1051, 1020, 966, 884, 764, 699, 620 cm⁻¹. Anal. Calcd for C₁₄H₁₅NO₅S: C, 54.36; H, 4.89; N, 4.53; S, 10.37. Found: C, 54.47; H, 4.94; N, 4.32; S, 10.29.

### (R)-3-Benzyloxycarbonyl-4-(phenylmethylthiomethyl)oxazolidin-5-one (33)

In a round-bottomed flask fitted with a Dean-Stark apparatus, a mixture of the S-benzyl cysteine (31b) (1.0 g, 2.9 mmol), paraformaldehyde (450 mg) and camphorsulfonic acid (50 mg) was suspended in benzene (30 ml). The mixture was heated to reflux (monitored by TLC for disappearance of starting material). The reaction mixture was then concentrated at reduced pressure. The residue was taken up in ethyl acetate and the organic layer was washed with saturated aqueous sodium bicarbonate solution to remove acidic material. The organic layer was dried (MgSO₄), filtered and evaporated in vacuo. The pale yellow syrupy residue was purified by column chromatography, eluting with 20% ether-hexane then 20-50% ethyl acetate-hexane to afford the oxazolidinone (33) as a clear colourless oil (920 mg, 89%). [α]_{D}²⁴ +102.3° (c 0.6, CHCl₃). ¹H NMR (300 MHz, CDCl₃) 7.34-7.20 (m, 10H), 5.50 (bs, 1H), 5.35 (d, 1H, J=4.1 Hz), 5.16 (s, 2H), 4.50 (bs, 1H), 3.69 (d, 1H, J_{AB}=13.3 Hz), 3.65 (d, 1H, J_{AB}=13.3 Hz), 3.37-2.89 (m, 2H). ¹³C NMR (75 MHz, CDCl₃) □ 171.28, 152.38, 137.45, 135.23, 128.95, 128.69, 128.55, 128.32, 127.28, 78.77, 68.01, 56.05, 37.24, 31.90, 31.40. IR (NaCl)ν 3086, 3062, 3030 and 3006 (CH, aromatic), 3000-2800 (CH, saturated), 1801 (C═O, oxazolidinone), 1717 (C═O, carbamate), 1495, 1452, 1413, 1357, 1290, 1257, 1212, 1165, 1127, 1052, 1019, 961, 764, 699 cm⁻¹. Anal. Calcd for C₁₉H₁₉NO₄S: C, 63.85; H, 5.36; N, 3.92. Found: C, 63.59; H, 5.62; N, 4.07.

### N-Benzyloxycarbonyl-N-methyl-S-phenylmethyl-L-cysteine (34)⁵⁶

The oxazolidinone (33) (850 mg, 2.4 mmol) was taken up in chloroform (20 ml). Triethylsilane (1.5 ml) was added followed by trifluoroacetic acid (20 ml) and the resulting mixture was left to stand for 2 d. The reaction mixture was concentrated under reduced pressure. The residue was diluted with excess saturated aqueous sodium bicarbonate solution. The aqueous phase was washed with ether and then acidified to pH 2 with 2 M hydrochloric acid. The acidic layer was then extracted with ether. The ethereal extracts were dried (MgSO₄) and then treated with dicyclohexylamine (2.4 mmol) and the solution was stored overnight at 0° C. The crystalline precipitate that formed was filtered off at the pump and dried to give the N-methyl-S-benzyl cysteine (34) as its DCHA salt (900 mg, 70%). Mp 105-107° C. [α]_{D}²⁶ −56.0° (c 1.0, CHCl₃). ¹H NMR (300 MHz, CDCl₃) (rotamers) 7.35-7.17 (m, 10H), 5.25-5.03 (m, 2H), 4.76 (dd, 1H, J=4.9, 10.6 Hz), 4.61 (dd, 1H, J=4.9, 10.5 Hz), 3.73-3.61 (m, 2H), 3.12-3.05 (m, 1H), 2.89-2.83 (m, 5H), 2.71-2.65 (m, 1H), 1.91-1.03 (m, 20H). ¹³C NMR (75 MHz, CDCl₃) (rotamers) δ 173.99, 173.66, 157.07 156.78, 138.49, 137.04, 136.85, 128.92, 128.73, 128.27, 127.70, 127.46, 126.69, 67.04, 66.83, 60.31, 59.79, 52.37, 36.17, 35.76, 32.21, 31.75, 30.62 30.29, 28.93, 28.81, 25.11, 24.67. IR (KBr disk)ν 3059 and 3029 (CH, aromatic), 3000-2800 (CH, saturated), 2525 and 2466 (H₂N⁺), 1692 (C═O, carbamate), 1624 (CO₂⁻), 1563, 1496, 1476, 1453, 1382, 1311, 1293, 1169, 1128, 1024, 760, 700 cm⁻¹. Anal. Calcd for C₃₁H₄₄N₂O₄S: C, 68.85; H, 8.20; N, 5.18; S, 5.93. Found: C, 68.91; H, 8.39; N, 5.05; S, 5.85.

### (4R,4′R)-3,3′-Bis-benzyloxycarbonyl-4,4′-[dithiobis(methylene)]bis-oxazolidin-5-one (36)

A mixture of the cystine carbamate (35) (3.0 g, 5.9 mmol), camphorsulfonic acid (40 mg), paraformaldehyde (2.0 g) and toluene (100 ml) was heated to reflux (ca. 1.5 h, TLC). The reaction mixture was then concentrated under reduced pressure and the residue was filtered through a short column or plug of silica gel eluting with dichloromethane. The filtrate was concentrated in vacuo and the residual syrup was refrigerated at −5° C. overnight to initiate crystallisation. The mixture of syrup and most of the solid was taken up in hot ether solution (small amounts of ethyl acetate can be added to facilitate dissolution). The solution was concentrated by boiling to ca. 15 ml and then hexane (10 ml) was added. The solution was left to stand overnight at 0° C. The precipitate that formed was filtered off at the pump and dried to give the oxazolidinone (36) as a crystalline solid (1.05 g, 33%). Mp 86-88° C. [α]_{D}²³ +99.4° (c 1.0, CHCl₃). ¹H NMR (300 MHz, CDCl₃) 7.34-7.29 (m, 10H), 5.49-5.46 (m, 2H), 5.30 (bs, 2H), 5.15-5.12 (m, 4H), 4.51 (brs, 2H), 3.47-3.12 (m, 4H). ¹³C NMR (75 MHz, CDCl₃) δ 170.62, 152.15, 135.19, 128.66, 128.42, 78.43, 68.04, 55.07, 38.86, 37.85. IR (KBr disk)ν 3090, 3065, and 3033 (CH, aromatic), 3000-2800 (CH, saturated), 1796 (C═O, oxazolidinone), 1704 (C═O, carbamate), 1500, 1453, 1431, 1362, 1296, 1268, 1213, 1175, 1159, 1125, 1055, 761, 700 cm⁻¹. Anal. Calcd for C₂₄H₂₄N₂O₈S₂: C, 54.12; H, 4.54; N, 5.26; S, 12.04. Found: C, 54.11; H, 4.46; N, 5.17; S, 11.96.

### Attempted Reductive Cleavage of the Cystine Oxazolidinone (36)

The cystine oxazolidinone (36) (300 mg, 0.6 mmol) was taken up in chloroform (5 ml). Triethylsilane (750 μl) was added followed by trifluoroacetic acid (5 ml) and the reaction mixture was left to stand for 2 d. Work-up of the reaction mixture as described for the N-methyl cysteine (33) afforded the thiazolidine (30) as an oil (241 mg, 80%) identical in all respects to material previously reported.^{28, 56}

### Example 3

### Methionine

The methionine carbamate reacts well to form the oxazolidinone (29b) (FIG. 3), but the reductive cleavage was not successful and gave a mixture of products. This was attributed to the sidechain thioether acting as a cation scavenger (FIG. 4); a phenomenon, which is known in peptide chemistry through the use of dimethyl sulfide.⁴¹

[image]

As with cysteine, the nucleophilicity of the thiomethyl group needed to be ameliorated to prevent its participation in the reductive cleavage. The corresponding sulfoxide (38)⁴² (98%) was easily prepared (Scheme 15) by reaction of the oxazolidinone (29b) with m-chloroperoxybenzoic acid (mCPBA). Initial attempts to convert the methionine carbamate (28b) to its sulfoxide⁴³ were successful but the subsequent oxazolidination was compromised by its poor solubility. The sulfoxide (38) was then reductively cleaved in high yield (92%) to give the N-methyl amino acid (39). It was evident this procedure caused a small amount of deoxygenation of the sulfoxides (38) or (39) and so the procedure included treatment with hydrogen peroxide to reoxidize the thioether (40). The N-methyl methionine (40) was formed in 81% yield in a one-pot procedure, which included the ammonium iodide/dimethyl sulfide treatment.

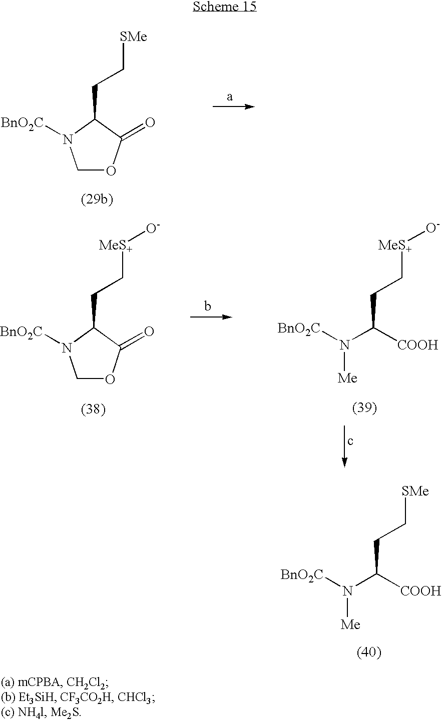

### (S)-3-Carbonylbenzyloxy-4-(2-methanesulfinylethyl)-oxazolidin-5-one (38)⁴²

To a solution of the methionine oxazolidinone (29b) (3.0 g, 10.2 mmol) in dichloromethane (135 ml) was slowly added m-CPBA (1.74 g) and the reaction mixture was stirred at room temperature for 15 min. The solution was washed with sodium carbonate solution (3×40 ml, 10% w/v). The aqueous washings were extracted with dichloromethane (2×50 ml) and the combined organic layers were dried (MgSO₄), filtered and concentrated in vacuo to give the sulfoxide (35) as a clear colourless gum. ¹H NMR (300 MHz, CDCl₃) 7.32 (s, 5H), 5.48-5.47 (m, 1H), 5.22-5.08 (m, 3H), 4.38 (t, 1H, J=6.0 Hz), 2.75 (brs, 2H), 2.47 (s, 3H), 2.38-2.27 (m, 2H). ¹³C NMR (75 MHz, CDCl₃) δ 171.09, 152.89, 152.83, 135.00, 128.61, 128.33, 77.74, 68.13, 53.79, 53.68, 49.28, 38.54, 38.47, 24.39, 24.08. IR (NaCl)ν 3038 (CH, aromatic), 3000-2900 (CH, saturated), 1796 (C═O, oxazolidinone), 1714 (C═O, carbamate), 1502, 1413, 1356, 1317, 1247, 1132, 1049, 753 cm⁻¹. HRMS calcd for C₁₄H₁₇NO₅S (M⁺) 311.0827, found 311.0832.

### N-Benzyloxycarbonyl-N-methyl-L-methionine-d-sulfoxide (39a) and N-Benzyloxycarbonyl-N-methyl-L-methionine-l-sulfoxide (39b)

To a solution of the sulfoxides (38) (1.3 g, 4.2 mmol) in chloroform (22 ml) was added triethylsilane (2.0 ml) and trifluoroacetic acid (22 ml). The reaction mixture was stirred at room temperature for 2 d and it was then concentrated at reduced pressure. The residue was taken up in ethyl acetate and extracted with sodium carbonate solution (10% w/v, 4×15 ml). The combined aqueous extracts were washed with ethyl acetate and then acidified with 5 M hydrochloric acid. The aqueous layer was then extracted with dichloromethane (3×20 ml) and the combined organic extracts were dried (MgSO₄), filtered and concentrated in vacuo. The residue (1.22 g) was taken up in methanol (12 ml). To the methanolic solution was added concentrated hydrochloric acid (20 μl). 30% Hydrogen peroxide was added dropwise until TLC indicated the presence of a single compound. The reaction mixture was concentrated at reduced pressure and the residue was taken up in dichloromethane and washed with water. The dichloromethane phase was then dried (MgSO₄), filtered and evaporated in vacuo. The residue (1.22 g) was recrystallised from ethyl acetate-ether to give the sulfoxide (39a) as a solid (210 mg, 16%). Mp 145-148° C. [α]_{D}²⁵ +21.0° (c 1.0, MeOH). ¹H NMR (300 MHz, CDCl₃) (rotamers) [(D₆)DMSO) 7.39-7.28 (m, 5H), 5.10-5.02 (m, 2H), 4.60-4.53 (m, 1H), 2.82-2.70 (m, 3H), 2.67-2.58 (m, 2H), 2.51-2.47 (m, 3H), 2.22-2.20 (m, 1H), 2.06-1.97 (m, 1H) ¹³C NMR (75 MHz, CDCl₃) (rotamers) δ171.81, 156.08, 155.59, 136.72, 128.43, 128.36, 127.84, 127.38, 66.48, 58.44, 49.99, 38.11, 31.86, 31.30, 22.18, 21.50. IR (KBr). ν 3600-3200 (CO₂H), 3063 and 3031 (CH, aromatic), 3000-2800 (CH, saturated), 1721 (CO, acid), 1691 (CO, carbamate), 1629, 1492, 1456, 1407, 1366, 1303, 1222, 1146, 987 cm⁻¹. Anal. Calcd for C₁₄H₁₉NO₅S: C, 53.66; H, 6.11; N, 4.47; S, 10.23. Found, C, 53.56; H, 6.25; N, 4.39; S, 10.35. The mother liquor was concentrated at reduced pressure to afford the sulfoxide 39b as a colorless gum (1.00 g, 76%). [α]_{D}²⁵ −53.2° (c 1.0, MeOH). ¹H NMR (300 MHz, CDCl₃) (rotamers) [(D6)DMSO) 7.37-7.29 (m, 5H), 5.10-5.03 (m, 2H), 4.63-4.56 (m, 1H), 2.83-2.70 (m, 4H), 2.59-2.49 (m, 4H), 2.25-2.20 (m, 1H), 2.10-1.97 (m, 1H). ¹³C NMR (75 MHz, CDCl₃) (rotamers) δ171.87, 156.12, 155.65, 136.81, 128.44, 128.37, 127.81, 127.38, 66.50, 57.94, 57.76, 49.68, 49.48, 37.83, 31.62, 31.15, 21.54, 21.23. IR (NaCl)ν 3500-3200 (COOH), 3063 and 3023 (CH, aromatic), 3000-2800 (CH, saturated), 1700 (CO, acid), 1550, 1455, 1404, 1317, 1222, 1169, 1132, 1001, 823, 742, 693 cm⁻¹. HRMS calcd for C₁₄H₁₉NO₅S (M⁺) 314.1062 found 314.1074.

### N-Benzyloxycarbonyl-N-methyl-L-methionine (40)⁵⁷

To a solution of the sulfoxides (38) (1.3 g, 4.2 mmol) in chloroform (22 ml) was added triethylsilane (2.0 ml) and trifluoroacetic acid (22 ml). The reaction mixture was stirred at room temperature for 2 d. The solution was then cooled to 0° C. and ammonium iodide (3.02 g) and dimethylsulfide (1.53 ml) were added. The reaction mixture was stirred vigorously for 1 h at 0° C. and then it was diluted with toluene and evaporated at reduced pressure. The residue was taken up in ether and extracted with sodium carbonate solution (10% w/v, 4×15 ml). The combined aqueous extracts were washed with ether and then acidified to pH 2 with 5 M hydrochloric acid. The aqueous layer was then extracted with dichloromethane (3×20 ml) and the combined organic extracts were washed with 5% sodium thiosulfate solution, dried (MgSO₄), filtered and concentrated in vacuo to give the methionine (40) as a clear colourless oil (1.01 g, 81%). For analytical purposes this material can be taken up in ether and treated with dicyclohexylamine (1 eq.) to give the DCHA salt. Mp 97-99° C. [α]_{D}²³ −17.0° (c 1.0, CHCl₃). ¹H NMR (300 MHz, CDCl₃) (rotamers) 9.47 (brs, 2H), 7.34-7.22 (m, 5H), 5.15-4.95 (m, 2H), 4.59-4.51 (m, 1H), 2.88-2.82 (m, 5H), 2.53-2.22 (m, 3H), 2.03 and 1.99 (2×s, 3H), 1.90-1.02 (m, 21H). ¹³C NMR (75 MHz, CDCl₃) (rotamers) δ174.68, 174.60, 156.83, 136.96, 136.83, 128.18, 127.56, 127.38, 66.78, 66.65, 60.44, 60.16, 52.29, 31.56, 30.54, 30.15, 29.70, 29.66, 28.94, 28.69, 25.07, 24.61, 15.40. IR (KBr) ν 3037 (CH, aromatic), 3000-2800 (CH, saturated), 2525 and 2452 (NH₂⁺), 1702 (CO, carbamate), 1631 (CO₂⁻), 1546, 1517, 1483, 1440, 1390, 1320, 1268, 1170, 1121, 1062, 740 cm⁻¹. Anal. Calcd for C₂₆H₄₂N₂O₂S: C, 65.24; H, 8.84; N, 5.85. Found, C, 65.32; H, 8.54; N, 5.99.

### Example 4

### Asparagine

Although it has been shown that carbamoylation of the sidechain of glutamine allows its conversion to N-methyl glutamine²⁸, this protection strategy was not possible with asparagine and so an alternative was sought. Tritylation (Trt) of the asparagine amide sidechain was achieved under acidic conditions (Scheme 16).⁴⁵ Carbamoylation with N-(benzyloxycarbonyloxy)succinimide (BnOCO₂Succ) then gave the precursor (41)⁴⁵, and subsequent oxazolidination afforded (42) (83%). The solubility of the asparagine carbamate (41) was not high and a minimal amount of DMF was included in the reaction protocol to improve substrate solubility and reaction yield. Reductive cleavage of the oxazolidinone (42) gave an 86% yield of the desired N-methyl product (43). In this reaction the N-methyl group forms with concomitant removal of the trityl group under the acidic conditions. The low solubility of the N-methyl intermediate (43) necessitated workup by concentration of the reaction mixture and column chromatography of the residue rather than the normal aqueous procedure.

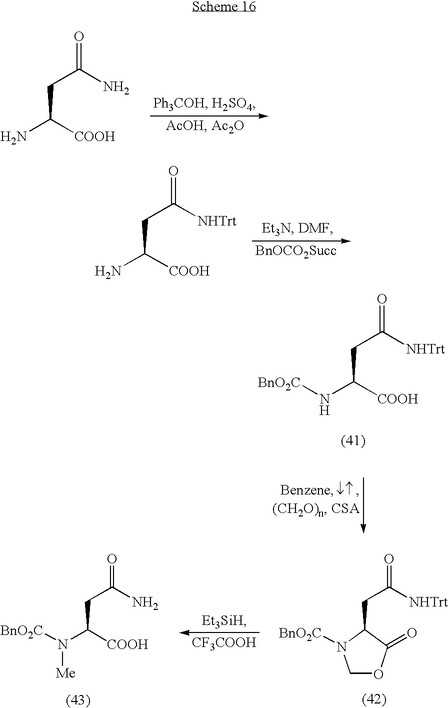

### (S)-3-Carbonylbenzyloxy-4-(triphenylmethylaminoacetoyl)-oxazolidin-5-one (42)

The carbamate (41) (2.54 g, 5.0 mmol) was dissolved in a minimum of DMF (ca. 2-3 ml). The solution was then added to toluene (120 ml), followed by camphorsulfonic acid (50 mg) and paraformaldehyde (5 g). The mixture was heated to reflux until the reaction was complete, ca. 2 h (monitored by TLC, 40% ethyl acetate-hexane). The reaction mixture was concentrated under reduced pressure and the residue was taken up in ethyl acetate and the organic layer was washed with saturated aqueous sodium bicarbonate solution to remove acidic material. The organic layer was dried (MgSO₄), filtered and evaporated in vacuo. The residue was purified by column chromatography, eluting with 40% ethyl acetate-hexane to afford the oxazolidinone (42) as a foam (2.16 g, 83%). A sample of the foam was recrystallised from hot ether-ethyl acetate to give a solid. Mp 122-123° C. [□]_{D}²³+60.3° (c 1.0, CHCl₃). ¹H NMR (300 MHz, CDCl₃) 7.36-7.04 (m, 20H), 6.77 and 6.53 (2m, 1H), 5.46-4.89 (m, 3H), 4.63-4.20 (m, 2H), 3.30-2.92 (m, 2H). ¹³C NMR (75 MHz, CDCl₃) (rotamers) 8171.68, 167.90, 152.40, 144.03, 135.33, 128.63, 128.48, 128.27, 127.95, 127.04, 77.83, 77.45, 70.95, 67.70, 37.79, 36.82. IR (KBr disk) ν3352 (CONH), 3088, 3060, 3031 and 3007 (CH, aromatic), 3000-2800 (CH, saturated), 1797 (C═O, oxazolidinone), 1710 (C═O, carbamate), 1685 (C═O, amide), 1519, 1494, 1449, 1417, 1360, 1319, 1256, 1210, 1165, 1130, 1055, 755, 721, 700 cm⁻¹. Anal. Calcd for C₃₂H₂₈N₂O₅: C, 73.83; H, 5.42; N, 5.38. Found: C, 73.94; H, 5.39; N, 5.24.

### N-Benzyloxycarbonyl-N-methyl-L-asparagine (43)⁵⁸

The oxazolidinone (42) (1.0 g, 1.9 mmol) was dissolved in chloroform (12 ml) and to this solution was added triethylsilane (1.2 ml) followed by trifluoroacetic acid (12 ml) and the reaction mixture was left to stir at room temperature for 2 d. The reaction mixture was concentrated in vacuo and the residue was chromatographed on silica eluting with 90:10:0.5 chloroform-methanol-water. The appropriate fractions were combined and concentrated under reduced pressure. The residue was triturated with ether to give the N-methyl asparagine (43) as a colourless solid (458 mg, 86%). Mp 134-136° C. [α]_{D}²³ −60.8° (c 1.0, MeOH). ¹H NMR (300 MHz, CD₃OD) 7.34-7.25 (m, 5H), 5.11 (s, 2H), 4.89-4.82 (m, 1H), 2.97-2.89 (m, 4H), 2.79-2.66 (m, 1H). ¹³C NMR (75 MHz, CD₃OD) (rotamers) δ 175.33, 175.11, 173.66, 158.09, 137.96, 137.76, 129.52, 129.05, 128.98, 128.68, 68.68, 68.45, 59.11, 58.53, 36.79, 36.29, 34.05, 33.95. IR (KBr disk)ν 3500-3200 (CO₂H), 3427 and 3219 (CONH₂), 3115, 3092, 3067, 3033 and 3009 (CH, aromatic), 3000-2800 (CH, saturated), 1714 (CO₂H), 1679 (C═O, carbamate), 1590, 1484, 1451, 1403, 1370, 1340, 1256, 1228, 1201, 1169, 1011, 773, 739 cm⁻¹. Anal. Calcd for C₁₃H₁₆N₂O₅: C, 55.71; H, 5.75; N, 9.99. Found: C, 55.65; H, 5.83; N, 9.93.

### Example 5

### Arginine and Homoarginine

The guanidine group of arginine presents several problems for the oxazolidinone chemistry. But N-methyl arginine is an attractive target given the key role arginine plays in many enzymic transformations. The lysine carbamate (44) was readily available and so the sequence in Scheme 17 leading to the N-methyl-lysine (53) was investigated as a trial for the preparation of N-methyl homoarginine. Diazotisation of the carbamate (44) and its decomposition with sodium acetate led to the formation of the acetate (45) as a mixture with the elimination product (46). These compounds were not separated prior to oxazolidination. Oxazolidination of the mixture gave the expected oxazolidinones (47) and (48), which were separated by column chromatography.

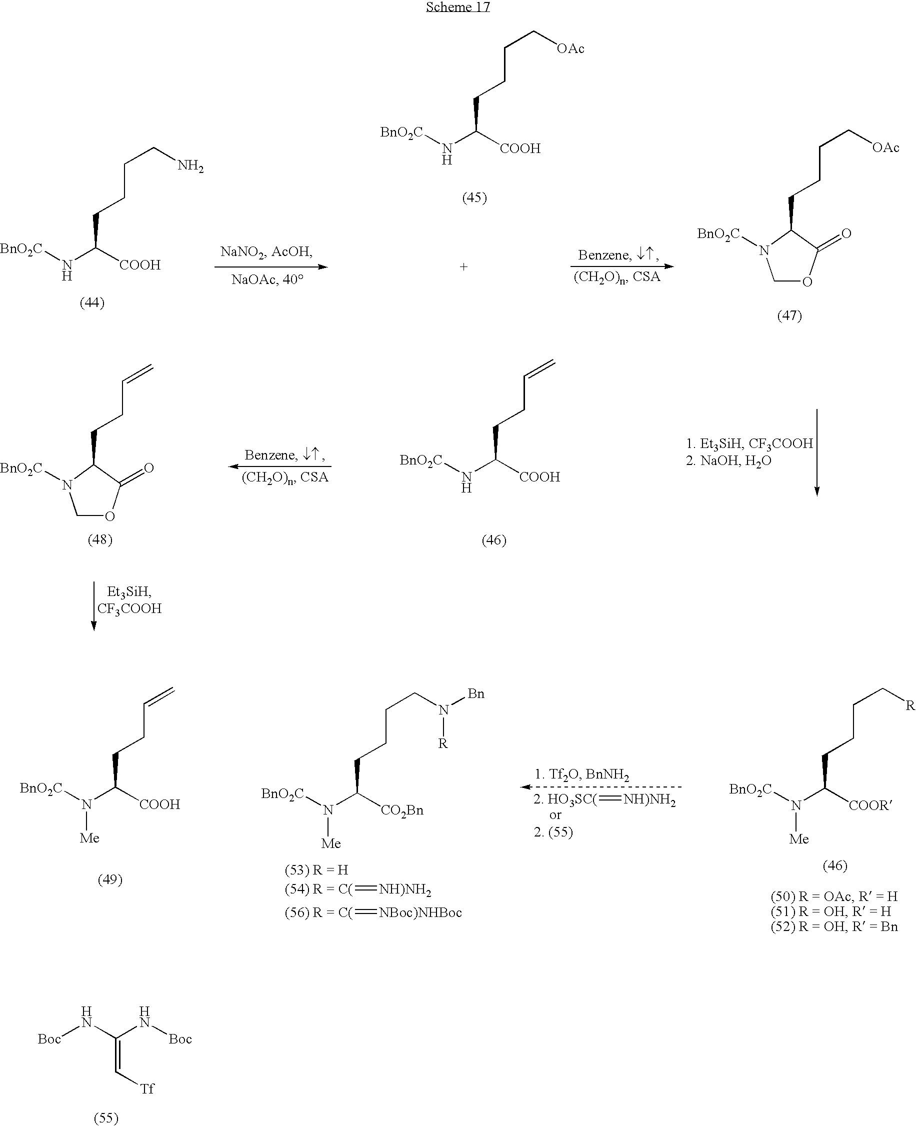

Reductive cleavage of the butenyl oxazolidinone (48) gave the expected N-methyl amino acid (49) (64%). Reductive cleavage of the oxazolidinone (47) afforded the N-methyl compound (50) (82%). Then the acetate group was hydrolysed with aqueous base to give the alcohol (51) and the carboxylic acid was esterified to give the benzyl ester (52). Treatment of the benzyl ester (52) with triflic anhydride formed the triflate ester in situ. Benzylamine was added to the triflate and displacement provided the fully protected N-methyl lysine (53). The secondary amine (53) was then treated with aminoiminomethanesulfonic acid⁴⁶ but this failed to afford the N-methyl homoarginine (54). In addition, reaction with the triflylguanidine (55)⁴⁷ also failed to give the desired homoarginine (56). It was evident the secondary amine was insufficiently nucleophilic for these guanylation reactions. A similar sequence with ornithine intermediates also failed for the same reasons.

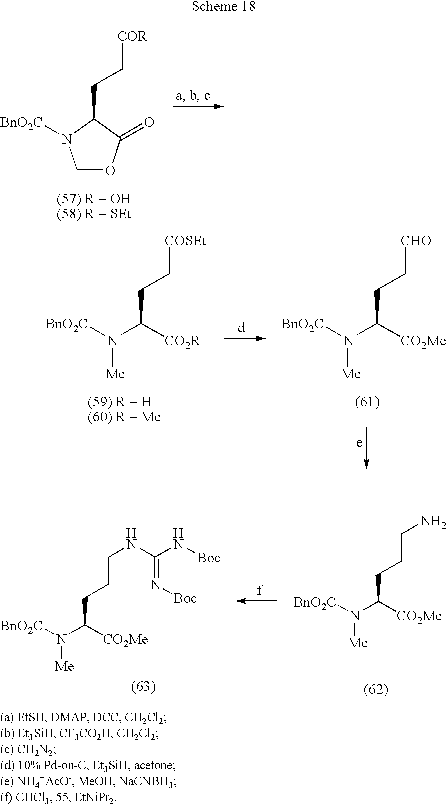

The reactions depicted in Scheme 18 were then pursued, which offered a synthesis of N-methyl arginine via direct and less demanding transformations. The glutamic oxazolidinone (57) was converted to the thioester (58) (92%) by DCC coupling with ethanethiol. Reductive cleavage then proceeded smoothly to give the N-methyl amino acid (59) (87%). The carboxylic acid was protected as the methyl ester (60) via diazomethylation⁴⁸ in quantitative yield. The resulting thioester was converted into the aldehyde (61) by treatment with palladium catalyst in the presence of triethylsilane.⁴⁹ This material was not purified but was submitted directly to the next series of reactions for generating the target arginine. Reductive amination with ammonium acetate then afforded the N-methyl ornithine (62). Reaction of this with the guanylating reagent (55) gave the desired N-methyl arginine (63) in 49% yield from the methyl ester (60).

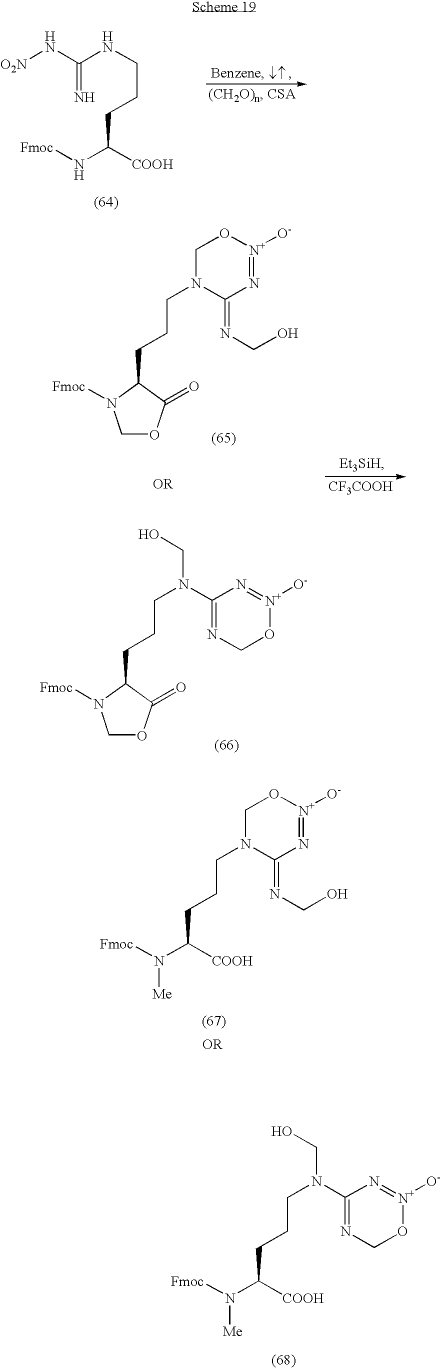

In addition, conversion of the commercial Fmoc L-nitroarginine (64) was attempted. The oxazolidination reaction did not give the expected compound. Electrospray mass spectrometry and NMR analysis indicated the product had a molecular weight of 495, which required the presence of extra methylene groups. It is proposed that the novel heterocycle (66) was prepared (Scheme 19). Similar chemistry on nitroguanidino compounds in which there is a second nucleophilic reagent, a primary amine, included in the reaction results in intermolecular condensation of the guanidine, the amine and two equivalents of the formaldehyde.⁵⁰ It is proposed in the current reaction that there is no second nucleophilic reactant and so the weakly nucleophilic nitro group is able to intercept a reactive iminium intermediate and form the isolated product.

There are potentially two possible routes that the reaction can take; either to produce initially either (65) or (66) and then (67) or (68). It was shown that the reaction proceeded via (66) from the detailed analysis of the NMR spectra and comparison with the data expected for structure (65). Initially the NMR spectra of compound (65) were run in CDCl₃; however, broad peaks in both the ¹H and the ¹³C NMR spectra were seen as a result of conformational mobility. Thus, in this solvent there were a number of missing peaks in the 2D spectra. In DMSO at 333K it was shown that the peaks were sharper and provided satisfactory 2D spectra.

An accurate assignment of all the protons and carbons in the molecule was obtained using a combination of COSY, DEPT, HSQC and the HMBC experiments. The complete assignment is presented in Table 1.

TABLE 1
¹³C and ¹H NMR data of compound (66) at 300 MHz,
333° K in DMSO.

| | Carbon | Proton | Number of | | J |
|---|---|---|---|---|---|
| Carbon | Shift | Shift | Protons | Multiplicity | coupling |
| *2 | 77.34 | 5.30, 5.22 | 2 | dd | 19.95, 4.06 |
| 4 | 53.98 | 4.05 | 1H | t | 6.3 |
| 5 | 171.95 | | | | |
| 1′ | 26.69 | 1.58-1.39 | 2H | m | |
| 2′ | 22.17 | 1.58-1.39 | 2H | m | |
| 3′ | 44.79 | 3.24-3.18 | 2H | m | |
| 5′ | 77.34 | 4.84 | 2H | s | |
| 4′ | 153.84 | | | | |
| *6′′ | 72.99 | 4.90-4.89 | 2 | d | 1.2 |
| 1′′′ (8′′′) | 126.85 | 7.63-7.61 | 2H | d | 7.13 |
| 2′′′ (7′′′) | 124.59 | 7.31 | 2H | t | 7.34 |
| 3′′′ (6′′′) | 119.73 | 7.39 | 2H | t | 7.31 |
| 4′′′ (5′′′) | 127.39 | 7.86-7.84 | 2H | d | 7.42 |
| 4a′′′ (4b′′′) | 143.41 | | | | |
| 8a′′′ (9a′′′) | 140.62 | | | | |
| 9′′′ | 46.49 | 4.28 | 1H | t | 5.6 |
| 10′′′ | 66.57 | 4.54 | 2H | m | |
| 11′′′ | 152.44 | | | | |
| OH | | 9.6 | 1 | s | |

*Overlapping signals

The HMBC experimental data were critical in differentiating between structures (65) and (66). Long-range correlations from the (C4′) at δ153.84 to the protons of (H3′) (δ3.24-3.18), (H5′) (δ4.84) and (H6″) (δ4.90-4.89) would be seen in both. The assignment as 66 was determined from the long-range correlation between the (C3′) at (δ44.79) and the (H5′) at δ4.48 of the γ-position of the propyl chain and the hydroxymethyl group, marked B in FIG. 4. This is not possible in structure 65.

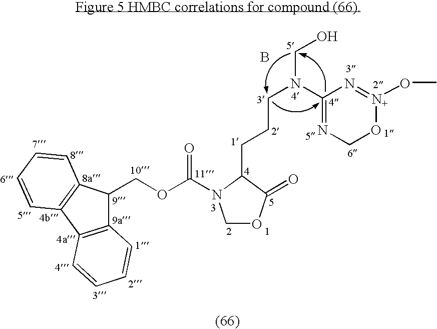

The conclusion depends on the accurate assignment of the carbons and protons for the 1′, 2′ and 3′ positions. Proton assignments for these positions were obtained from the mCOSY experiment, and HSQC and HMBC experiments were used for the carbon assignments.

The reductive cleavage produces a single product that has a molecular weight of 467 (ESMS). The ¹H and ¹³C NMR spectra clearly indicate the presence of the N-methyl group and a methylene group associated with the oxatriazine. The reduction of the oxazolidinone (66) to the acid (68) shows the disappearance of the H2 proton peaks at δ5.35 and appearance of the expected NCH₃ at δ2.72 indicating that only the oxazolidinone ring is reductively cleaved. It is apparent that the triethylsilane/trifluoroacetic acid is able to reduce the 5-oxazolidinone but not the new heterocyclic ring formed from the nitroguanidine.

### Preparation of the lysine derived oxazolidinones (47) and (48)

Deamination via diazotisation of the lysine carbamate (44) (1.01 g, 3.1 mmol) according to the method of Hutton⁵⁹ afforded the acetate (48) and the alkene (46) as a mixture (1.0 g) which, was not purified. The crude acetate (45) was taken up in benzene (30 ml) and camphorsulfonic acid (35 mg) and paraformaldehyde (3 g) were added. The mixture was heated to reflux for 2 h and then allowed to cool. The mixture was concentrated at reduced pressure and the residue was taken up in ethyl acetate and the organic layer was washed with saturated aqueous sodium bicarbonate solution to remove acidic material. The organic layer was dried (MgSO₄), filtered and evaporated in vacuo. The residue was purified by column chromatography, eluting with 30% ethyl acetate-hexane to afford firstly, the oxazolidinone (46) as a clear colourless oil (113 mg, 13%). [α]_{D}²⁵ +112.6° (c 1.0, CHCl₃). ¹H NMR (300 MHz, CDCl₃) 7.34-7.29 (m, 5H), 5.70 (brs, 1H), 5.49 (brs, 1H), 5.31-4.95 (m, 5H), 4.31 (brs, 1H), 2.12-1.59 (m, 4H). ¹³C NMR (75 MHz, CDCl₃) (rotamers) δ172.10, 152.68, 136.36, 135.35, 128.58, 128.50, 128.19, 127.69, 115.79, 77.77, 67.80, 67.67, 55.16, 54.96, 54.24, 29.60, 28.43. IR (NaCl) ν3076 and 3034 (CH, aromatic), 3000-2800 (CH, saturated), 1801 (C═O, oxazolidinone), 1716 (C═O, carbamate), 1506, 1413, 1357, 1316, 1251, 1164, 1128, 1050, 919, 754, 693 cm⁻¹. Anal. Calcd for C₁₅H₁₇NO₄: C, 65.44; H, 6.22; N, 5.09. Found: C, 65.42; H, 6.31; N, 5.07. Further elution gave the oxazolidinone 47 as a colorless oil (478 mg, 46%). [α]_{D}²⁵ +86.6° (c 1.0, CHCl₃). ¹H NMR (300 MHz, CDCl₃) 7.33 (s, 5H), 5.48 (brs, 1H), 5.23-5.09 (m, 3H), 4.27 (t, 1H, J=5.2 Hz), 3.97 (t, 2H, J=6.2 Hz), 2.04-1.76 (m, 2H), 1.98 (s, 3H), 1.43-1.32 (m, 4H). ¹³C NMR (75 MHz, CDCl₃) δ172.03, 170.80, 152.78, 135.29, 128.56, 128.49, 128.16, 77.80, 67.80, 63.71, 54.64, 30.18, 27.99, 20.79, 20.76. IR (NaCl) ν3067 and 3033 (CH, aromatic), 3000-2800 (CH, saturated), 1801 (C═O, oxazolidinone), 1724 (2×C═O), 1506, 1413, 1361, 1318, 1244, 1167, 1131, 1047, 755, 696 cm⁻¹. Anal. Calcd for C₁₇H₂₁NO₆: C, 60.89; H, 6.31; N, 4.18. Found: C, 60.80; H, 6.41; N, 4.26.

### (S)—N-Benzyloxycarbonyl-N-methyl-2-(3-butenyl)-glycine (49)

The oxazolidinone (48) (310 mg, 1.1 mmol) was taken up in chloroform (6 ml) and triethylsilane (540 μl) was added followed by trifluoroacetic acid (6 ml) and the mixture was left to stand at room temperature for 2 d. The reaction mixture was diluted with toluene and then concentrated in vacuo and the residue was taken up in ether and extracted with aqueous sodium carbonate solution (4×2 ml). The combined aqueous extracts were washed with ether and then acidified to ˜pH 2 with 5 M hydrochloric acid. The aqueous phase was then extracted with dichloromethane (3×5 ml). The organic phase was dried (MgSO₄), filtered and evaporated to give a yellow oil (230 mg). The oil was chromatographed on silica eluting with 94:5.5:0.5 chloroform-methanol-water to provide the N-methyl amino acid (49) as a clear colourless oil (200 mg, 64%). [α]_{D}²⁴ −16.6° (c 1.0, CHCl₃). ¹H NMR (300 MHz, CDCl₃) (rotamers) 10.02 (brs, 1H), 7.34-7.30 (m, 5H), 5.86-5.50 (m, 1H), 5.19-4.63 (m, 5H), 2.89-2.88 (m, 3H), 2.12-1.60 (m, 4H). ¹³C NMR (75 MHz, CDCl₃) (rotamers) δ176.66, 157.14, 156.45, 136.64, 136.47, 136.32, 136.15, 128.39, 127.97, 127.80, 127.68, 116.03, 115.85, 67.61, 58.06, 57.80, 31.02, 30.63, 30.10, 29.95, 27.96 and 27.69. IR (NaCl). ν3600-3000 (COOH), 3077 and 3038 (CH, aromatic), 3000-2800 (CH, saturated), 1705 (C═O), 1548, 1451, 1402, 1321, 1210, 1153, 1035, 916, 854, 740, 692 cm⁻¹. HRMS calcd for C₁₅H₁₉NO₄ (M+H) 278.1392 found 278.1384.

### (S)—N-Benzyloxycarbonyl-N-methyl-2-(4-acetoxybutanyl)-glycine (50)

The oxazolidinone (47) (3.26 g, 9.7 mmol) was taken up in dichloromethane (50 ml) and triethylsilane (5.0 ml) was added followed by trifluoroacetic acid (50 ml) and the mixture was left to stand at room temperature for 2 d. The reaction mixture was concentrated in vacuo and the residue was taken up in aqueous sodium bicarbonate solution, and washed with ether. The aqueous was then acidified 5 M hydrochloric acid and extracted with dichloromethane. The organic phase was dried (MgSO₄), filtered and evaporated to give a yellow oil (2.69 g, 82%) which was used directly in the next step.

### (S)—N-Benzyloxycarbonyl-N-methyl-2-(4-hydroxybutanyl)-glycine (51)

The crude acetate (50) (1.67 g, 4.9 mmol) was treated with 1 M sodium hydroxide solution (10.8 ml) at 0° C. and left to stir at that temperature for 1.5 h. The solution was then acidified with dilute hydrochloric acid and extracted with chloroform (6×30 ml). The combined extracts were dried (MgSO₄) and evaporated in vacuo. The residue was triturated with ether to afford the alcohol (51) as a colourless solid (1.2 g, 83%). Mp 122-124° C. [α]_{D}²⁴ −22.3° (c 1.0, acetone). 1H NMR [300 MHz, CD₃COCD₃] (rotamers) 7.38-7.31 (m, 5H), 5.14-5.12 (m, 2H), 4.76 (dd, 0.5H, J=4.7, 10.9 Hz), 4.65 (dd, 0.5H, J=4.7, 10.7 Hz), 3.55 (t, 2H, J=4.9 Hz), 2.89-2.87 (m, 3H), 1.95-1.35 (m, 6H). ¹³C NMR (75 MHz, CDCl₃) (rotamers) δ172.99, 157.51, 156.85, 138.06, 129.18, 128.56, 128.34, 67.49, 62.14, 59.03, 32.94, 31.05, 30.57, 29.45, 29.06, 23.37. IR (KBr disk). ν3600-3200 (CO₂H and OH), 3095 and 3030 (CH, aromatic), 3000-2800 (CH, saturated), 1738 (C═O, acid), 1650 (C═O, carbamate), 1490, 1405, 1322, 1258, 1206, 1162, 1101, 1024, 763 cm⁻¹. Anal. Calcd for C₁₅H₂₁NO₅: C, 61.00; H, 7.17; N. 4.74. Found: C, 60.87; H, 7.34; N, 4.65.

### (S)—N-Benzyloxycarbonyl-N-methyl-2-(4-hydroxybutanyl)-glycine benzyl ester (52)

The acid (51) (300 mg, 1.0 mmol) was dissolved in dimethylformamide (10 ml). Anhydrous potassium carbonate (210 mg) was added and the mixture was vigorously stirred while benzyl bromide (121 μl) was added. The resulting mixture was stirred at room temperature under a nitrogen atmosphere overnight. It was then diluted with water (150 ml) and extracted with ethyl acetate (3×20 ml) and the combined extracts were dried (MgSO₄) filtered and evaporated at reduced pressure to give the benzyl ester (52) as a clear gum (334 mg, 87%). A sample was further purified by column chromatography eluting with 30% ethyl acetate-hexane to give the pure ester (52). [α]_{D}²⁵ −23.4° (c 1.0, CHCl₃). ¹H NMR (300 MHz, CDCl₃) (rotamers) 7.32-7.24 (m, 10H), 5.18-5.08 (m, 4H), 4.85 (dd, 0.5H, J=4.9, 10.8 Hz), 4.62 (dd, 0.5H, J=4.9, 10.5 Hz), 3.59-3.51 (m, 2H), 2.86-2.83 (m, 3H), 2.04-1.30 (m, 6H). ¹³C NMR (75 MHz, CDCl₃) (rotamers) δ171.34, 171.38, 156.99, 156.23, 136.46, 136.33, 135.49, 135.38, 128.45, 128.35, 128.16, 127.94, 127.79, 127.58, 67.33, 66.67, 62.22, 58.64, 58.36, 31.85, 30.87, 30.21, 28.57, 28.18, 22.24, 22.16. IR (NaCl) ν3600-3200 (OH), 3094, 3065 and 3036 (CH, aromatic), 3000-2800 (CH, saturated), 1739 (C═O, ester), 1699 (C═O, carbamate), 1456, 1401, 1320, 1257, 1212, 1151, 1069, 909, 742, 693 cm⁻¹. Anal. Calcd for C₂₂H₂₇NO₅: C, 68.55; H, 7.06; N, 3.63. Found: C, 68.28; H, 7.24; N, 3.72.

### N^{α}-Benzyloxycarbonyl-N^{α}-methyl-N^{ε}-benzyl-L-lysine benzyl ester (53)

The alcohol (52) (740 mg, 1.9 mmol) was dissolved in dry dichloromethane (9 ml) and the solution was cooled to −50°. Triethylamine (460 μl) was added followed by trifluoromethanesulfonic anhydride (490 μl). After 15 min at −50° TLC analysis indicated complete conversion to the corresponding triflate. Benzylamine (0.82 ml) was then added in one portion at −50° and the reaction mixture was stirred at this temperature for 30 min and then at room temperature overnight. The reaction mixture was diluted with ether (100 ml) and the organic phase was washed with water (3×300 ml). The organic phase was dried (MgSO₄), filtered and concentrated at reduced pressure. The crude residue was purified by column chromatography eluting firstly, with 60% ethyl acetate-hexane and then 8% methanol-ethyl acetate to afford the lysine (53) as a clear oil (701 mg, 78%).

### (S)-3-Carbonylbenzyloxy-4-(2-ethylsulfanylcarbonyl-ethyl)-oxazolidin-5-one (58)

To a sample of the glutamic acid oxazolidinone (57) (2.0 g, 6.8 mmol) in dichoromethane (8 ml) was added ethanethiol (1.01 ml, 13.6 mmol) and DMAP (20 mg) and the solution was cooled to 0° C. DCC (1.69 g, 8.2 mmol) was added in one portion and the reaction mixture was stirred at 0° C. for 30 min. Acetic acid (0.8 ml) was then added and stirring was continued for 10 min. The mixture was diluted with ether (50 ml) and suction filtered. The filtrate was washed sequentially with 10% sodium carbonate solution (2×20 ml), water, 0.5M hydrochloric acid (20 ml), water and brine. The ethereal solution was then dried (MgSO₄), filtered and concentrated in vacuo to give the thioester (58) as an oil (2.13 g, 92%). A sample was further purified for analytical purposes by column 20 chromatography on silica eluting with 50% ether-hexane. [α]_{D}²⁵ +99.2° (c 1.0, CHCl₃). ¹H NMR (300 MHz, CDCl₃) 7.30 (s, 5H), 5.42 (brs, 1H), 5.14 (d, 1H, J=4.5 Hz), 5.12 (s, 2H), 4.27 (t, 1H, J=5.7 Hz), 2.79 (q, 2H, J=7.4 Hz, 2H), 2.65-2.49 and 2.36-2.11 (2m, 4H), 1.16 (t, 3H, J=7.4 Hz). ¹³C NMR (75 MHz, CDCl₃) δ197.34, 171.36, 152.64, 135.12, 128.39, 128.29, 128.04, 77.59, 67.72, 53.71, 38.49, 25.95, 23.10, 14.44. IR (NaCl) ν3097, 3063 and 3033 (CH, aromatic), 3000-2800 (CH, saturated), 1800 (C═O, oxazolidinone), 1718 (C═O, carbamate and thioester), 1500, 1412, 1356, 1317, 1252, 1169, 1131, 1052, 998, 840, 756, 696 cm⁻¹. Anal. Calcd for C₁₆H₁₉NO₅S: C, 56.96; H, 5.68; N, 4.15. Found: C, 56.72; H, 5.57; N, 4.30.

### (S)-2-(Benzyloxycarbonyl-methyl-amino)-4-ethylsulfanylcarbonyl-butyric acid (59)

A sample of the oxazolidinone (58) (1.0 g, 0.3 mmol) was dissolved in dichloromethane (15 ml) and triethylsilane (1.4 ml) was added followed by trifluoroacetic acid (15 ml) and the mixture was left to stand for 3 d at room temperature. The solution was then taken up in toluene (50 ml) and evaporated to dryness under reduced pressure. The residue was then taken up in ether and extracted with 10% sodium carbonate solution. The aqueous layer was washed with ether and then acidified to ˜pH 2 with 5M hydrochloric acid. The aqueous phase was then extracted with dichloromethane (4×20 ml). The combined extracts were dried (MgSO₄), filtered and evaporated in vacuo. The residual oil (920 mg) slowly crystallised. The oil was recrystallised from ether-hexane to afford the carboxylic acid (59) as a colourless solid (680 mg, 87%). Mp 94-96° C. [α]_{D}²⁴ −15.6° (c 1.0, CHCl₃). ¹H NMR (300 MHz, CDCl₃) 10.22 (brs, 1H), 7.32-7.27 (m, 5H), 5.14 (s, 2H), 4.85-4.56 (m, 1H), 2.88-2.81 (m. 5H), 2.65-2.05 (m, 4H), 1.21 (t, 3H, J=7.4 Hz). ¹³C NMR (75 MHz, CDCl₃) (rotamers) δ8198.17, 175.29, 157.11, 156.27, 136.36, 128.50, 128.08, 127.83, 67.84, 58.46, 40.31, 31.47, 24.49, 23.37, 14.57. IR (KBr disk). ν3700-3200 (CO₂H), 3134, 3097, 3069 and 3033 (CH, aromatic), 3000-2800 (CH, saturated), 1736, 1687 and 1648 (3×C═O), 1492, 1455, 1411, 1374, 1325, 1254, 1222, 1174, 1096, 1069, 1017, 989, 767, 739 cm⁻¹. Anal. Calcd for C₁₆H₂₁NO₅S: C, 56.62; H, 6.24; N, 4.13. Found: C, 56.75; H, 6.30; N, 4.29.

### (S)-2-(Benzyloxycarbonyl-methyl-amino)-4-ethylsulfanylcarbonyl-butyric acid methyl ester (60)

The title compound (60) was prepared by diazomethylation of the carboxylic acid (59) by the standard method.⁴⁸ The methyl ester (60) was isolated in 100% yield. [α]_{D}²⁴ −21.8° (c 2.0, CHCl₃). ¹H NMR (300 MHz, CDCl₃) (rotamers) 7.26-7.17 (m, 5H), 5.06-5.04 (m, 2H), 4.67 and 4.49 (2dd, 1H, J=5.0, 10.5 Hz), 3.59-3.52 (m, 3H), 2.77-2.71 (m, 5H), 2.58-2.40 and 2.31-1.91 (2m, 4H), 1.12 (t, 3H, J=7.4 Hz). ¹³C NMR (75 MHz, CDCl₃) (rotamers) δ197.68, 170.71, 170.59, 156.42, 155.65, 136.18, 136.03, 128.08, 127.63, 127.49, 127.34, 67.09, 57.90, 51.79, 39.96, 39.68, 31.32, 30.57, 24.29, 23.99, 22.92, 14.34. IR (NaCl). ν3095, 3063 and 3029 (CH, aromatic), 3000-2800 (CH, saturated), 1743 and 1700 (3×C═O), 1448, 1403, 1316, 1219, 1180, 1141, 1057, 1007, 907, 742, 695. Anal. Calcd for C₁₇H₂₃NO₅S: C, 57.77; H, 6.56; N, 3.96. Found: C, 58.05; H, 6.74; N, 4.15.

### (S)-2-(Benzyloxycarbonyl-methyl-amino)-5-[tert-butoxycarbonylamino-(tert-butoxycarbonylimino)methyl)-pentanoic acid methyl ester (63)

To a sample of the thioester (60) (200 mg, 0.56 mmol) in acetone (1.0 ml) was added triethylsilane (300 μl) followed by 10% Palladium-on-charcoal catalyst (50 mg) and the reaction mixture was stirred vigorously for 1 h. The mixture was filtered through celite and the filtrate was concentrated under reduced pressure. The residue aldehyde (61) was purified by chromatography on a short silica column eluting with 20% ethyl acetate-hexane to remove the triethylsilane. The fractions collected were concentrated in vacuo and the residue was taken up in methanol (4 ml) and ammonium acetate (222 mg) was added followed by sodium cyanoborohydride (71 mg) and the mixture was stirred at room temperature for 30 min. The solution was concentrated at reduced pressure to about 1 ml and it was then diluted with saturated aqueous sodium bicarbonate solution (10 ml). The aqueous phase was then extracted with dichloromethane (3×5 ml). The combined extracts were dried (MgSO₄), filtered and concentrated in vacuo to give the primary amine (62). The amine (62) was taken up in chloroform (filtered through neutral alumina, 2 ml) and di-boc-triflylguanidine (221 mg, 0.56 mmol) was added followed by diisopropylethylamine (0.15 ml, 0.85 mmol) and the mixture was stirred at room temperature for 2 h. The solution was concentrated under reduced pressure and the residue was purified by chromatography on silica eluting with chloroform. The material isolated was further purified by chromatography eluting with 50% ether-hexane to give the protected N-methyl arginine (63) as a clear colourless oil (149 mg, 49%). [α]_{D}¹⁹ −13.0° (c 0.5, CHCl₃). ¹H NMR (300 MHz, CDCl₃) (rotamers) 11.45 (brs, 1H), 8.28 (brs, 1H), 7.31-7.23 (m, 5H), 5.12 (d, 1H, J_{AB}=12.3 Hz), 5.07 (d, 1H, J_{AB}=12.3 Hz), 4.76 and 4.57 (2dd, 1H, J=4.8, 10.5 Hz), 3.65-3.58 (m, 3H), 3.43-3.33 (m, 2H), 2.82 (s, 3H), 2.02-1.37 (m, 22H). ¹³C NMR (75 MHz, CDCl₃) (rotamers) δ171.53, 171.35, 163.30, 156.81, 156.00, 153.12, 136.41, 136.31, 128.84, 128.32, 127.84, 127.56, 82.98, 79.07, 67.34, 58.37, 58.19, 51.99, 40.17, 39.97, 31.07, 30.26, 28.12, 27.89, 26.12, 25.84, 25.73. IR (NaCl). ν3335 and 3290(sh), 2×NH, 3133, 3104, 3076 and 3033 (CH, aromatic), 3000-2800 (CH, saturated), 1712 and 1633 (4×C═O), 1574, 1446, 1411, 1364, 1327, 1238, 1229, 1141, 1053, 866, 809, 762, 746 cm⁻¹. Anal. Calcd for C₂₆H₄₀N₄O₈: C, 58.19; H, 7.51; N, 10.44. Found: C, 58.32; H, 7.56; N, 10.22.

### 4S-}4-(4-[4-Hydroxymethylimino-2-oxy-4H-(1,2,3,5)-oxatriazin-5-yl]-propyl}-oxazolidin-5-one-3-carboxylic acid 9H-fluoren-9-ylmethyl ester (66)

The nitroarginine carbamate (64) (1.0 g, 2.3 mmol) was dissolved in toluene (50 ml) in a round-bottomed flask fitted for reflux. To the solution was added camphorsulfonic acid (10 mg) and paraformaldehyde (1.5 g) and the mixture was heated to reflux for 1.5 h. The reaction mixture was cooled and the solvent was decanted from residual solid material. The solvent was concentrated in vacuo and the residue was purified by column chromatography eluting with 80% ethyl acetate-dichloromethane to afford the oxazolidinone (66) as a colourless foam (750 mg, 67%). [α]_{D}³³ +117.8° (c 1.0, CH₂Cl₂). 1H NMR [300 MHz, (D6)DMSO, 298K] 9.76 (s, 1H), 7.93-7.34 (m, 8H), 5.35 (s, 2H), 4.94-4.93 (m, 4H), 4.52 (brs, 2H), 4.32 (t, 1H, J=5.4 Hz), 3.57-3.44 (m, 2H), 3.27 (s, 1H), 1.90-1.25 (brs, 4H). ¹H NMR [300 MHz, (D₆)DMSO, 333K] 9.60 (s, 1H), 7.86-7.31 (m, 8H), 5.26 (dd, 2H, J=20.0, 4.1 Hz), 4.90 (d, 2H, J=1.2 Hz), 4.84 (s, 2H), 4.54 (m, 2H), 4.28 (t, 1H, J=5.6 Hz), 4.04 (t, J=6.4 Hz), 3.24-3.18 (m, 2H), 1.58-1.39 (m, 4H). ¹³C NMR [75 MHz, (D₆)DMSO, 298K] δ172.45, 153.74, 152.75, 143.67, 143.59, 140.87, 127.73, 127.21, 124.98, 120.14, 77.75, 77.55, 73.26, 66.82, 54.33, 46.61, 44.98, 26.87, 22.38. ¹³C NMR [75 MHz, (D₆)DMSO, 333K] δ171.95, 153.84, 152.44, 143.41 and 143.32, 140.62, 127.39, 126.85, 124.59, 124.56, 119.73, 77.34, 72.99, 66.57, 53.98, 46.49, 44.79, 26.69, 22.17. IR (KBr disk) ν3289 (OH), 3066, 3041, 3015 and 3007 (CH, aromatic), 3000-2800 (CH, saturated), 1798 (C═O, oxazolidinone), 1713 (C═O, carbamate), 1588, 1557, 1412, 1346, 1196, 1136, 1048, 940, 742, 709 cm⁻¹l. HRMS calcd for C₂₄H₂₆N₅O₇ (M+H) 498.1842 found 496.1816.

### 2S-2-[(9H-Fluoren-9-ylmethylmethoxycarbonyl)-methyl-amino]-5-[hydroxymethyl-(2-oxy-6H-[1,2,3,5]oxatriazin-4-yl-amino-pentanoic acid (68)

The oxazolidinone (66) (100 mg, 0.2 mmol) was dissolved in dichloromethane (4 ml) and triethylsilane (0.3 ml) was added followed by trifluoroacetic acid (4 ml) and the reaction mixture was stirred under a nitrogen atmosphere overnight. The mixture was concentrated at reduced pressure. The residue was purified by column chromatography eluting with 10% methanol-dichloromethane to afford the N-methyl compound (68) as a colourless foam (60 mg, 60%). [α]_{D}²² −12.9° (c 1.0, CH₂Cl₂). ¹H MMR [300 MHz, (D₆)DMSO, 300K] 9.60 (s, 1H), 7.84-7.26 (m, 8H), 4.90 (s, 2H), 4.88 (s, 2H), 4.35-3.97 (m, 4H), 3.30 (s, 2H), 2.72 (s, 3H), 1.70-1.40 (m, 4H). ¹³C NMR [75 MHz, (D₆)DMSO, 300K] δ171.97, 155.63, 153.90, 143.64 and 143.59, 140.54, 127.30, 126.76, 124.65, 119.70, 77.41, 73.04, 66.56, 57.94, 46.62, 44.95, 30.16, 25.10, 24.05. IR (KBr disk) ν 3700-2700 (COOH), 3300-3200 (═NH), 3064, 3039, 3018 and 3009 (CH, aromatic), 1739 (C═O), 1696 (C═O, carbamate), 1589, 1555, 1451, 1409, 1315, 1263, 1195, 1158, 1131, 1028, 992, 760, 741 cm⁻¹. HRMS calcd for C₂₄H₂₈N₅O₇ (M+H) 498.1989 found 498.1969.

### (S)-3-Carbonylbenzyloxy-4-(1-formyl-1H-indol-3-ylmethyl)-oxazolidin-5-one (72)

A mixture of the tryptophan carbamate (71) (3.0 g, 8.2 mmol), benzene (200 ml), camphorsulfonic acid (100 mg) and paraformaldehyde (5 g) was heated to reflux for 1.5 h. The reaction mixture was concentrated under reduced pressure and the residue was taken up in ether. The ethereal layer was washed with saturated aqueous sodium bicarbonate solution, dried (MgSO₄), filtered and concentrated in vacuo to give an oil. The oil was further purified by column chromatography, eluting with 60% ether-hexane to give the oxazolidinone (72) as a colourless foam (2.67 g, 86%). [α]_{D}²⁵ +154.0° (c 1.0, CHCl₃). ¹H NMR (300 MHz, CDCl₃) 9.31, 8.89 and 8.33-8.31 (2×brs and m, 2H), 7.58-7.04 (m, 9H), 5.21 (brs, 3H), 4.59-4.46 (m, 2H), 3.57-3.22 (m, 2H). ¹³C NMR (75 MHz, CDCl₃) δ171.73, 159.13, 155.53, 152.36, 135.25, 134.07, 130.74, 128.65, 125.40, 124.67, 124.27, 120.91, 119.68, 118.70, 116.69, 115.91, 109.49, 77.81, 67.86, 55.64, 26.11, 25.06. IR (KBr disk). ν 3100 and 3063 (CH, aromatic), 3000-2800 (CH, saturated), 1801 (C═O, oxazolidinone), 1712 (C═O, carbamate), 1604, 1459, 1417, 1370, 1241, 1198, 1163, 1127, 1047, 1001, 753, 696 cm⁻¹. Anal. Calcd for C₂₁H₁₈N₂O₅: C, 66.66; H, 4.79; N, 7.40. Found: C, 66.87; H, 5.06; N, 7.50.

### Example 6

### Tryptophan

Attempted oxazolidination of the carbamate of tryptophan results in decomposition. This is presumably due to side reactions of the indole nitrogen. An electron-withdrawing protecting group was anticipated to solve this problem and accordingly, the N-formyl tryptophan (70)⁵¹ (Scheme 20) was prepared in quantitative yield from L-tryptophan (69). Carbamoylation then gave the precursor (71) for oxazolidination. The oxazolidination proceeded in good yield (86%) and the oxazolidinone (72) was isolated as an oil. The following reductive cleavage did not proceed as planned. In all cases two products were isolated. The minor product was the expected N-methyl tryptophan (73). The major product was the β-carboline (74). The β-carboline arises by reaction of the intermediate iminium ion with the indole in an intramolecular electrophilic aromatic substitution. The resulting carboxylic acid (74) was isolated as its tert-butylammonium salt (75).

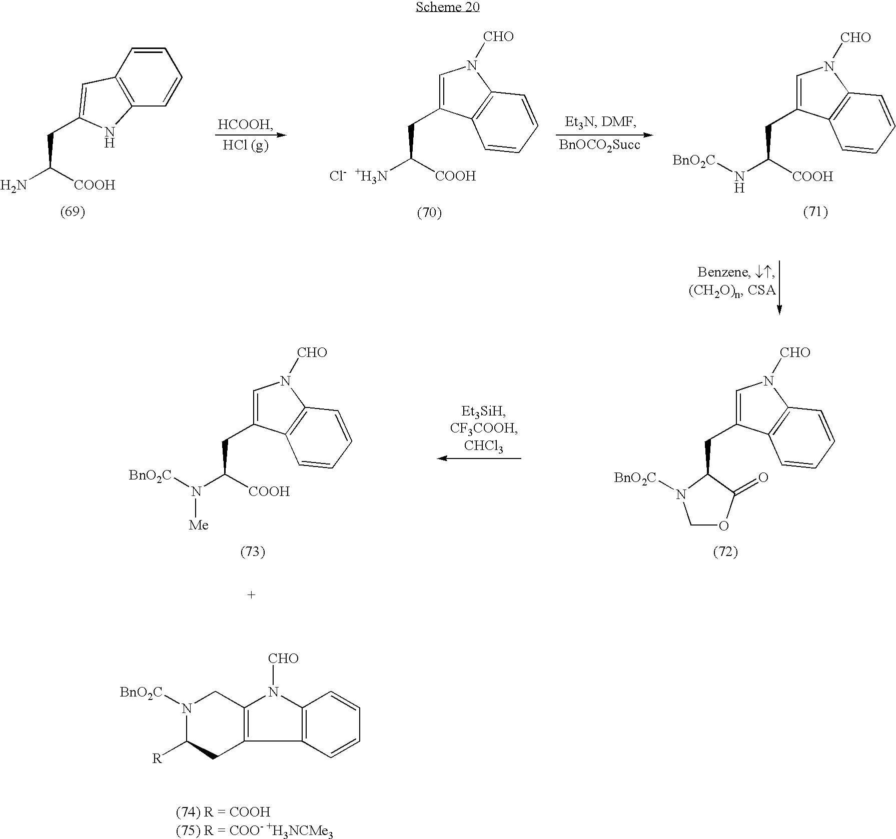

To further substantiate the role of the indole, the electrophilic aromatic substitution can be eliminated by reducing the pyrrole ring double bond. Accordingly, tryptophan (69) was converted to dihydrotryptophan (Scheme 21).⁵² This material underwent bis-carbamoylation to give the precursor (76). Oxazolidination proceeded smoothly to afford the mixture of diastereoisomers (77). The key reductive cleavage proceeded as expected to afford the N-methyl dihydrotryptophan (78) in 83% yield.

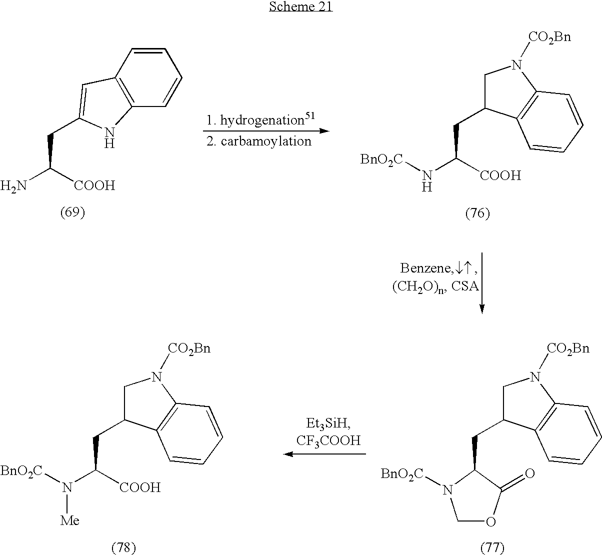

### (S)—N-Carbonylbenzyloxy-N-methyl-N′-formyl-L-tryptophan (73) and (S)-2-Carbonylbenzyloxy-9-formyl-1,3,4,9-tetrahydro-β-carboline-3-carboxylic acid (74)

To a mixture of the oxazolidinone (72) (500 mg, 1.3 mmol), chloroform (8 ml) and triethylsilane (0.6 ml) was added trifluoroacetic acid (8 ml) and the whole was left to stand at room temperature for 2 d. The mixture was then concentrated at reduced pressure and the residue was taken up in ether. The ethereal solution was extracted with saturated aqueous sodium bicarbonate solution (3×10 ml). The combined aqueous extracts were acidified with dilute hydrochloric acid and extracted with dichloromethane (3×20 ml). The extracts were dried (MgSO₄), filtered and evaporated at reduced pressure. The residue was purified by column chromatography eluting with 95:5:0.5:0.2 chloroform:methanol:water:acetic acid to give firstly, the β-carboline (74) as an oil (340 mg, 69%). The β-carboline can be converted to the tert-butylammonium salt (75) by taking it up in ether and adding an equivalent′of tert-butylamine. The precipitated tert-butylammonium salt (75) can be recrystallised from hot methanol. Mp 162-165° C. [α]_{D}²⁴ +41.3° (c 1.0, MeOH). ¹H NMR [300 MHz, (D₆)DMSO] 9.68, 9.32 and 8.21-7.93 (2×brs and m, 4H), 7.48-7.23 (m, 9H), 5.17-4.71 (m, 5H), 3.44-3.39 (m, 1H), 2.78-2.72 (m, 1H), 1.06 (s, 9H). ¹³C NMR (75 MHz, CDCl₃) (rotamers) δ172.56, 159.08, 155.94, 155.77, 152.97, 137.14, 136.06, 135.27, 130.34, 128.33, 127.70, 127.62, 127.42, 127.18, 123.75, 118.41, 114.77, 110.76, 66.07, 54.19, 50.06, 42.12, 27.19, 23.44, 23.21. IR (KBr disk). ν 3000-2800 (CH, saturated), 2743, 2636 and 2554 (NH₃⁺), 1711 (C═O, carbamate), 1637 (CO₂⁻), 1568, 1422, 1386, 1358, 1301, 1222, 1102, 1066, 748, 697 cm⁻¹. Anal. Calcd for C₂₅H₂₉N₃O₅: C, 66.50; H, 6.47; N, 9.31. Found: C, 66.67; H, 6.54; N, 9.20. Further elution afforded the N-methyl tryptophan 73 as a solid (110 mg, 22%)., Mp 129-130° C. [α]_{D}²⁵ −49.6° (c 0.5, CHCl₃). ¹H NMR (300 MHz, CDCl₃) (rotamers) 9.35, 8.83 and 8.38-8.36 (2×brs and m, 2H), 7.63-6.94 (m, 9H), 5.14-5.01 (m, 3H), 3.50-3.09 (m, 2H), 2.89-2.83 (m, 3H). ¹³C NMR (75 MHz, CDCl₃) (rotamers) δ175.25, 159.41, 156.88, 155.94, 136.29, 135.92, 134.33, 130.96, 128.52, 128.19, 127.79, 125.55, 124.89, 124.67, 124.21, 122.75, 119.75, 118.58, 116.26, 109.71, 67.83, 67.71, 58.66, 58.39, 31.97, 31.81, 24.68, 24.16. IR (KBr disk). ν 3600-3200 (CO₂H), 3091 and 3056 (CH, aromatic), 3000-2800 (CH, saturated), 1749 (C═O, acid), 1675 (CO, carbamate), 1605, 1459, 1392, 1319, 1251, 1191, 1135, 983, 795, 755, 699 cm⁻¹. Anal. Calcd for C₂₁H₂₀N₂O₅: C, 66.31; H, 5.30; N, 7.36. Found: C, 66.20; H, 5.39; N, 7.16.

### (S)-3-Carbonylbenzyloxy-4-[1-carbonylbenzyloxy-2,3-dihydroindol-3(R,S)-ylmethyl]-oxazolidin-5-one (77)

The dihydrotryptophan (76)²⁹ (2.0 g, 4.2 mmol) was dissolved in toluene (100 ml) and the solution was treated with camphorsulfonic acid (60 mg) and paraformaldehyde (5 g) and heated at reflux for 1 h. The clear solution was concentrated in vacuo and the residue was taken up in ethyl acetate and washed with saturated aqueous sodium bicarbonate solution. The organic layer was dried (MgSO₄), filtered and evaporated at reduced pressure to give a tan coloured oil (1.56 g). The oil was purified by column chromatography eluting with 20% ethyl acetate-hexane to give the oxazolidinone (77) as a colourless oil (1.38 g, 68%). ¹H NMR (300 MHz, CDCl₃) 7.89-6.93 (m, 14H), 5.53 (brs, 1H), 5.26-5.09 (m, 5H), 4.22-4.18 and 3.78-3.35 (2×m, 4H), 2.31-2.12 (m, 2H). ¹³C NMR (75 MHz, CDCl₃) 8171.71, 153.18, 152.78, 142.01, 136.16, 135.01, 132.87, 128.65, 128.49, 128.32, 128.12, 127.98, 123.86, 122.70, 114.84, 77.63, 68.21, 68.11, 66.92, 53.56, 53.16, 36.76, 36.03, 35.66. IR (NaCl). ν3000-2800 (CH, saturated), 1798 (C═O, oxazolidinone), 1712 (CO, carbamate), 1599, 1457, 1412, 1347, 1261, 1140, 1032, 752 cm⁻¹. Anal. Calcd for C₂₈H₂₆N₂O₆: C, 69.12; H, 5.39; N, 5.76. Found: C, 69.37; H, 5.67; N, 5.57.

### N,N′-bis-Carbonylbenzyloxy-3 (R,S)-3-[2(S)-2-carboxy-2-methylamino-ethyl]-N-methyl-2,3-dihydroindole (78)

To a solution of the dihydrotryptophan oxazolidinone (77) (1.2 g, 2.5 mmol) in chloroform (13 ml) was added triethylsilane (1.2 ml) and trifluoroacetic acid (13 ml). The mixture was left to stand for 2 d and it was then diluted with toluene and concentrated under reduced pressure. The greenish residue was chromatographed on a short silica gel column eluting with chloroform-methanol-water 93:6.5:0.5. The appropriate fractions were collected and the solvent was removed in vacuo. The residue was further purified by chromatography eluting with the same solvent system to give the N-methyl dihydrotryptophan (78) as a clear pale yellow oil (1.0 g, 83%). ¹H NMR (300 MHz, CDCl₃) 7.70-6.94 (m, 14H), 5.26-5.11 (m, 4H), 5.00-4.90 and 4.77-4.69 (2×m, 1H), 4.18-3.96 and 3.79-3.22 (2×m, 3H), 2.95-2.88 (m, 3H), 2.42-1.95 (m, 2H). ¹³C NMR (75 MHz, CDCl₃) δ174.75, 174.48, 157.03, 156.21, 152.87, 141.84, 136.16, 135.87, 133.36, 128.49, 128.43, 128.14, 128.03, 127.67, 124.38, 123.52, 122.78, 114.96, 67.77, 67.02, 57.03, 56.74, 53.21, 36.28, 34.54, 34.06, 31.07. IR (NaCl). ν3500-3200 (CO₂H), 3064 and 3038 (CH, aromatic), 3000-2800 (CH, saturated), 1703 (C═O), 1600, 1487, 1456, 1411, 1321, 1214, 1146, 1089, 1035, 971, 911, 856, 746, 697 cm⁻¹. HRMS calcd for C₂₈H₂₈N₂O₆ (M⁺) 488.1947 found 488.1944.

### Example 7

### Histidine

Again the basic and highly nucleophilic nature of the histidine sidechain caused problems in the initial attempts to form N-methyl histidine. Selective formation of the α-amino carbamate is difficult too. So the following sequence (Scheme 22) was adopted. Histidine methyl ester hydrochloride salt (79) was carbamoylated with two equivalents of (benzyloxycarbonyloxy)succinimide to give the bis-carbamate (60). Treatment of the bis-carbamate with propylamine effects removal of the imidazole carbamate. The reaction mixture was then evaporated under reduced pressure and the residue in acetonitrile was treated with 2,4-dinitrofluorobenzene, which undergoes a nucleophilic aromatic substitution to afford the dinitrophenyl (DNP) imidazole (81). Treatment of this compound with a mixture of acetic acid and 2M hydrochloric acid resulted in hydrolysis of the methyl ester to afford the acid as a hydrochloride salt (82).

This acid (82) is the precursor for the formation of the oxazolidinone, but standard conditions for its formation could not be used due to the insolubility of (82). This was overcome by dissolving the hydrochloride (82) in acetic acid and acetic anhydride in the presence of camphorsulfonic acid catalyst. Treatment of this mixture with paraformaldehyde afforded the required oxazolidinone (83) in high yield (>75%). Reductive cleavage then gave the N-methyl histidine carbamate (84) with the sidechain imidazole still protected with the dinitrophenyl group.

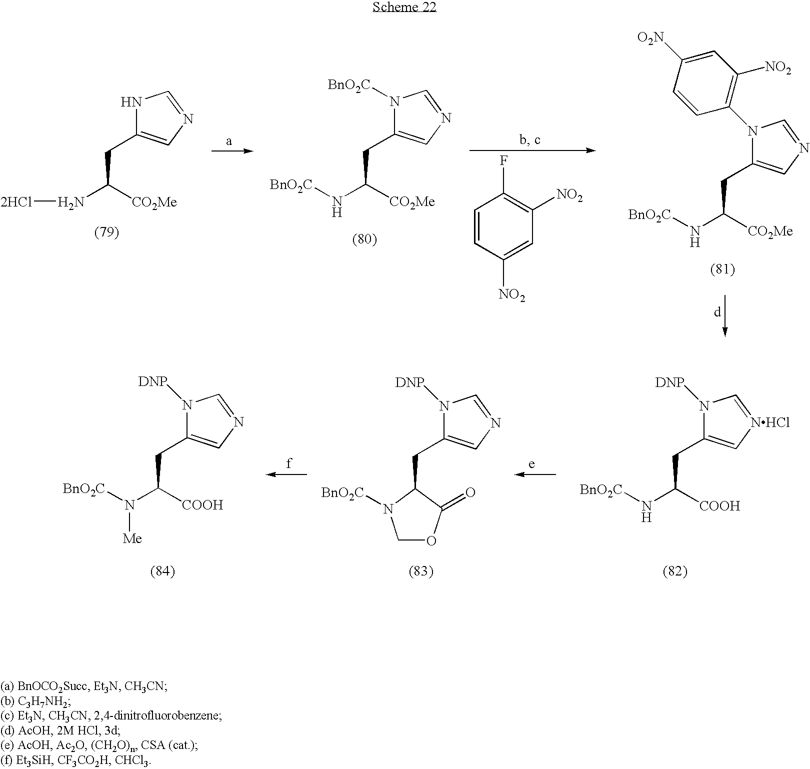

### N,N^{imid}-Biscarbonylbenzyloxy-L-histidine methyl ester (80)

A sample of the methyl ester (79) (1.0 g, 4.1 mmol) in acetonitrile (25 ml) was cooled to 0° with vigorous stirring and triethylamine (2.3 ml) was added followed by BnOCO₂-Succ (2.16 g, 8.7 mmol). The reaction mixture was stirred at 0° C. for 30 min and then at room temperature overnight. The solution was concentrated at reduced pressure and the residue was taken up in ethyl acetate and washed with water (3×25 ml). The organic phase was dried (MgSO₄), filtered and evaporated in vacuo to give a pale yellow oil (1.57 g). The oil was purified by column chromatography on silica eluting with 40% ethyl acetate-hexane to give the carbamate (80) as a colourless oil which slowly crystallised on standing (1.3 g, 72%). Mp 63-65° C. [α]_{D}²² +28.0° (c 1.0, CHCl₃). ¹H NMR (300 MHz, CDCl₃) 8.01 (s, 1H), 7.41-7.24 (m, 10 H), 7.17 (s, 1H), 6.10 (d, 1H, J=8.1 Hz), 5.35 (s, 2H), 5.07 (s, 2H), 4.67-4.61 (m, 1H), 3.68 (s, 3H), 3.12-2.99 (m, 2H). ¹³C NMR (75 MHz, CDCl₃) δ171.68, 155.83, 148.13, 138.76, 136.25, 133.78, 136.82, 129.04, 128.71, 128.58, 128.29, 127.89, 114.51, 69.74, 66.71, 53.36, 52.25, 29.80. IR (KBr disk). ν 3175, 3139, 3108 and 3040 (CH, aromatic), 3000-2800 (CH, saturated), 1755 (C═O, ester), 1695 (C═O, carbamate), 1531, 1447, 1409, 1257, 1015, 871, 736, 697 cm−₁. Anal. Calcd for C₂₃H₂₃N₃O₆: C, 63.15; H, 5.30; N, 9.61. Found: C, 63.35; H, 5.26; N, 9.78.

### N-Carbonylbenzyloxy-N^{imid}-(2,4-dinitrophenyl)-L-histidine methyl ester (81)

The bis-carbamate (80) (1.0 g, 2.3 mmol) was dissolved in propylamine (30 ml) and the solution was left to stir at room temperature for 1 h. The solvent was removed by evaporation at reduced pressure. The residue was taken up in ethyl acetate (100 ml) and the solution was again concentrated under reduced pressure. The residue was taken up in acetonitrile (20 ml) and triethylamine (0.64 ml) was added in one portion followed by 1-fluoro-2,4-dinitrobenzene (336 μl) and the solution was left to stir in the dark overnight. The solution was concentrated in vacuo and the residue was taken up in ethyl acetate and washed with water (3×50 ml). The organic layer was dried (MgSO₄), filtered and concentrated to provide a crude yellow oil (1.5 g). The oil was chromatographed on silica eluting with 88:10:2 dichloromethane-acetone-methanol to give the methyl ester (81) as a yellow gum (0.9 g, 84%). [α]_{D}²³ +23.7° (c 1.0, CHCl₃). ¹H NMR (300 MHz, CDCl₃) 8.80 (d, 1H, J=2.4 Hz), 8.53 (dd, 1H, J=2.4, 8.7 Hz), 7.76 (s, 1H), 7.67 (d, 1H, J=8.7 Hz), 7.31-7.24 (m, 5H), 6.84 (s, 1H), 6.12 (d, 1H, J=8.1 Hz), 5.07 (s, 2H), 4.69-4.66 (m, 1H), 3.71 (s, 3H), 3.15 (d, 2H, J=3.4 Hz). ¹³C NMR (75 MHz, CDCl₃) δ 171.71, 155.97, 146.98, 144.32, 139.15, 134.68, 136.46, 129.35, 128.43, 128.30, 128.05, 121.28, 117.44, 66.89, 53.48, 52.54, 29.85. IR (NaCl) ν3109, 3067 and 3021 (CH, aromatic), 3000-2800 (CH, saturated), 1717 (C═O), 1609, 1536 and 1346 (NO₂), 1449, 1255, 1214, 1055, 911, 835, 743 cm ⁻¹.

### N-Carbonylbenzyloxy-N^{imid}-(2,4-dinitrophenyl)-L-histidine hydrochloride salt (82)⁶¹

The methyl ester (81) (900 mg, 1.9 mmol) was dissolved in a mixture of glacial acetic acid (10 ml) and 2 M hydrochloric acid (10 ml) and the solution was left in the dark at room temperature for 3 d. The mixture was then concentrated at reduced pressure. The residue crystallised on standing and was purified by recrystallisation from methanol-ether to afford the hydrochloride salt (82) as a pale yellow solid (870 mg, 92%). Mp 169-171° C. [α]_{D}³⁰ −8.1° (c 1.0, MeOH). ¹H NMR [300 MHz, (D₆)DMSO] 9.48 (s, 1H), 9.00 (s, 1H), 8.81 (d, 1H, J=8.7 Hz), 8.15 (d, 1H, J=8.7 Hz), 7.81-7.78 (m, 2H), 7.38-7.27 (m, 5H), 5.03 (d, 1H, J_{AB}=12.5 Hz), 4.99 (d, 1H, J_{AB}=12.6 Hz), 4.46-4.39 (m, 1H), 3.28-3.05 (m, 2H). ¹³C NMR [75 MHz, (D₆)DMSO] δ172.20, 156.06, 148.11, 143.87, 136.83, 132.99, 131.86, 136.75, 131.52, 129.45, 128.34, 127.87, 127.69, 121.53, 120.56, 65.61, 52.90, 26.51. IR (KBr disk) ν3200-2500, (CO₂H), 3112 and 3064 (CH, aromatic), 3000-2800 (CH, saturated), 2604, (⁺NH Cl⁻), 1705 (C═O), 1614, 1542 and 1345 (NO₂), 1447, 1389, 1241, 1056, 911, 843, 745, 697, 633 cm⁻¹. Anal. Calcd for C₂₀H₁₈ClN₅O₈: C, 48.84; H, 3.69; N, 14.24. Found: C, 48.87; H, 3.83; N, 14.23.

### (S)-3-Carbonylbenzyloxy-4-[3H-3-(2,4-dinitrophenyl)-imidazol-4-ylmethyl]-oxazolidin-5-one (83)

To a solution of the carbamate (82) (200 mg, 0.4 mmol) in glacial acetic acid (5 ml) was added camphorsulfonic acid (10 mg), acetic anhydride (50 μl) and paraformaldehyde (50 mg). The mixture was heated with stirring at 85° C. for 2.5 h under a nitrogen atmosphere. The mixture was cooled to room temperature and then concentrated at reduced pressure. The residue was taken up in ethyl acetate and washed with aqueous sodium carbonate solution (10% w/v, 3×20 ml). The ethyl acetate phase was dried (MgSO₄), filtered and evaporated to dryness. The residual material was further purified by column chromatography eluting with ethyl acetate to give the oxazolidinone (83) as a yellow foam (133 mg, 66%). [α]_{D}²⁵ +148.3° (c 1.0, CHCl3). 1H NMR (300 MHz, CDCl3) 8.81 (d, 1H, J=2.5 Hz), 8.54 (dd, 1H, J=2.5, 8.7 Hz), 7.66 (d, 1H, J=8.7 Hz), 7.62 (s, 1H), 7.32-7.24 (m, 5H), 6.84-6.64 (m, 1H), 5.37 (brs, 1H), 5.26-5.10 (m, 2H), 4.89 (d, 1H, J=3.7 Hz), 4.51-4.49 (m, 1H), 3.49-3.37 (m, 1H), 3.18 (dd, 1H, J=2.4, 14.9 Hz.). 13C NMR (75 MHz, CDCl₃) δ 172.03, 152.31, 146.95, 144.35, 138.27, 135.64, 134.88, 136.69, 129.36, 128.53, 128.39, 128.29, 121.24, 117.95, 78.10, 67.69, 54.61, 28.57, 27.77. IR (KBr disk) ν 3110 (CH, aromatic), 3000-2800 (CH, saturated), 1800 (C=O, oxazolidinone), 1714 (C=O, carbamate), 1611, 1540 and 1352 (NO₂), 1503, 1419, 1253, 1132, 1051, 748, 699 cm⁻¹. Anal. Calcd for C₂₁H₁₇N₅O₈: C, 53.96; H, 3.67. Found: C, 53.82; H, 3.72.

### (S)—N-Carbonylbenzyloxy-N-methyl-N′-(2,4-dinitrophenyl)-L-histidine (84)

To a solution of the oxazolidinone (83) (460 mg, 1.0 mmol) in chloroform (5 ml) was added triethylsilane (470 μl) and trifluoroacetic acid (5 ml) and the reaction mixture was left to stand for 2 d. The solution was then concentrated under reduced pressure. The residue was taken up in a minimum of 95% chloroform-methanol and the precipitate, which formed, was filtered off at the pump to give the N-methyl amino acid (84) (225 mg). The filtrate was concentrated in vacuo and the residue was purified by column chromatography eluting with 92:7.5:0.5 chloroform-methanol-water to afford the N-methyl amino acid (84) (150 mg). The combined solids were recrystallised from methanol-ether to give the title compound (84) as a solid (375 mg, 81%). Mp 165-167°C. [α]_{D}²⁵ −24.7° (c 1.0, MeOH). ¹H NMR [300 MHz, (D₆)DMSO] (rotamers) 8.92-8.91 (m, 1H), 8.65-8.62 (m, 1H), 7.98 (brs, 1H), 7.92-7.88 (m, 1H), 7.28-7.19 (m, 6H), 5.04-4.95 (m, 2H), 4.88-4.79 (m, 1H), 3.13-2.99 (m, 2H), 2.82-2.79 (m, 3H). ¹³C NMR (75 MHz, CDCl₃) (rotamers) δ 172.14, 155.80, 155.47, 146.22, 143.52, 139.72, 136.85, 134.63, 137.03, 129.36, 128.69, 128.32, 127.67, 127.21, 127.11, 121.32, 117.06, 116.94, 66.29, 66.18, 58.93, 58.79, 31.83, 31.64, 27.61, 27.14. IR (KBr disk) ν 3600-3200 (CO₂H), 3185, 3130 and 3041 (CH, aromatic), 3000-2800 (CH, saturated), 1734 (C═O, acid), 1680 (C═O, carbamate), 1618, 1545 and 1347 (CNO₂), 1492, 1460, 1402, 1308, 1187, 1143, 1087, 842 cm⁻¹. Anal. Calcd for C₂₁H₁₉N₅O₈: C, 53.73; H, 4.08; N, 14.92. Found: C, 53.55; H, 4.07; N, 14.65.

### Example 8

### Proline

Due to the tertiary substitution of the α-amino nitrogen in N-methyl proline there was limited interest in its synthesis as it can not be readily incorporated in peptide sequences except at the N-terminus. The formation of the proline oxazolidinone (88) has been reported⁵³ though its synthesis is not high yielding. The isolation of the oxazolidinone (85) can be avoided by the simple expedient of combining aqueous formaldehyde and proline (86) in methanol (Scheme 23). This mixture was then subjected to hydrogenating conditions to afford the N-methyl proline (87) in near quantitative yield. This approach was employed by Lin et al.⁵⁴ to prepare an N-methyl proline ester from a proline ester.

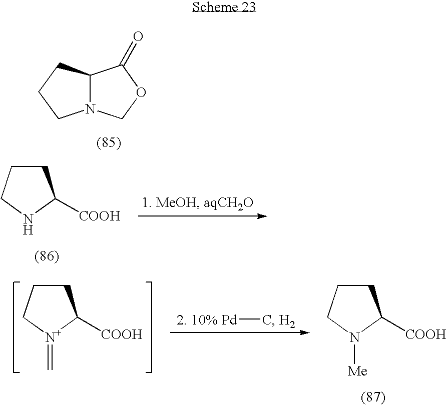

### N-methyl-L-proline (87)

L-Proline (86) (2.0 g, 17.4 mmol) was dissolved in methanol (20 ml) and to this solution was added 40% aqueous formaldehyde solution (1.4 ml, 19.1 mmol). This was followed by the addition of 10% palladium-on-charcoal catalyst (500 mg) and the resulting slurry was stirred in a hydrogen atmosphere overnight. The slurry was then filtered through a Celite pad to remove the catalyst. The pad was washed with methanol and the combined filtrates were concentrated under reduced pressure. The residue was taken up in ethanol-benzene (1:1, 100 ml) and concentrated a second time to provide a solid, which was recrystallised from methanol-diethyl ether. In this way N-methyl proline (87) was isolated as fine needles (2.2 g, 98%). Mp 142-145° C. [α]_{D}²³ −78.0° (c 2.0, MeOH). ¹H NMR (300 MHz, D₂O) 3.71-3.65 and 3.55-3.51 (2m, 1H), 3.00-2.91 (m, 1H), 2.74 (s, 3H), 2.34-2.28 (m, 1H), 1.99-1.78 (m, 3H). ¹³C NMR (75 MHz, CDCl₃) δ 173.06, 70.18, 55.83, 40.26, 28.34, 22.37. IR (KBr disk) ν 3000-2800 (CH, saturated), 2675 and 2605 (ammonium ion), 1669 (CO₂H), 1612 (CO₂⁻), 1468, 1401, 1354, 1327, 1234, 1183, 1112, 1056, 1025, 808, 775 cm⁻¹. HRMS calcd for C₆H₁₁NO₂ (M⁺) 129.0790 found 129.0784.

### Summary of Examples 1 to 8

5-oxazolidinone chemistry has been applied to the 20 common α-amino acids (and some others) in the formation of N-methyl derivatives, it is possible to classify the compounds according to their ease of manipulation. In the first group are those α-amino acids with sidechains that do not interfere with the oxazolidination and subsequent reductive cleavage. Into this group fits glycine, alanine, valine, leucine, isoleucine, phenylalanine, aspartic acid, glutamic acid, proline, tyrosine and phenylglycine. Historically, it is these-amino acids that have been concentrated on by other workers.⁵³ Methionine gives one of the highest yields of the corresponding 5-oxazolidinone but does not react well in the reductive cleavage. The second category includes those α-amino acids for which a simple sidechain protection reaction that is also compatible with standard solid phase deprotection conditions allows their participation in the oxazolidinone chemistry. These amino acids are serine, threonine, cysteine, tyrosine, lysine, asparagine, glutamine and ornithine. Tyrosine has been included in both categories because while the N-methylation sequence works in moderate to low yield without the phenolic hydroxyl protected, sidechain benzylation substantially improves the yield. The third category is those amino acids that require devoted synthetic schemes and more exotic functional group protection. This group currently consists of the problematic α-amino acids arginine, homoarginine, histidine, tryptophan and methionine.

### Example 9

### Dipeptides

A number of dipeptides that would be suitable for peptide incorporation have been prepared. They are not the most ideal examples as solid phase peptide synthesis methods most frequently use Fmoc and Boc protection of the N-terminus. The N-methyl amino acid chemistry is compatible with these groups, but the early development work has been entirely with Cbz protection on the nitrogen. This Cbz group works well in synthesis for standard solution phase approaches.

Preparation of N-Carbonylbenzyloxy-L-Leu-L-N-Methyl-Val-tert-Butyl Ester

R²=isopropyl

R=isobutyl

R″=benzyl

The N-methyl valine ester (500 mg, 1.3 mmol) was dissolved in dry dichloromethane (4 ml) and stirred under a nitrogen atmosphere at 0° C. The leucine acid (1.2 eq, 1.6 mmol, 421 mg) was added followed by PyBroP (1.2 eq, 1.6 mmol 746 mg) and DIPEA (4.0 eq, 5.3 mmol, 927 μl). The mixture was stirred for 3 h at 0° C. while being monitored by TLC. The mixture was then passed through a celite pad and the cake was washed with methanol. The filtrate was concentrated at reduced pressure and the residue was passed through silica gel, eluting with 20% ethyl acetate-hexane to give the title dipeptide as a slightly opaque, pungent smelling residue (576 mg, 91%); [α]²⁵_{D}=−61.1 (c, 0.99 in CH₂Cl₂); vₘₐₓ (NaCl)/cm⁻¹ 3299 (NH), 2963 (Aryl CH), 2874 (saturated CH), 1728, 1648 (C═O), 1528, 1463, 1368, 1252, 1161, 1047, 740, 698; ¹H NMR (300 MHz, CDCl₃) (rotamers) δ 7.34-7.27 (5 H, m, ArH), 5.60-5.28 (1 H, m, NH), 5.08 (2 H, s, PhCH₂), 4.79-4.69 (2 H, m, 2×α-H), 3.03-2.90 (3 H, m, NCH₃), 2.36-2.08 [1 H, m, CH(CH₃)₂], 1.86-1.61 [1 H, m, (CH₃)₂CH], 1.44 [9 H, s, (CH₃)₃], 1.07-0.82 (12 H, m, CH₃×4); ¹³C NMR (75 MHz, CDCl₃) (rotamers) δ 173.79, 169.79 and 156.14 (3×C═O), 136.48 (Aryl C), 128.39, 127.95, 127.90, 127.81, 127.40 and 126.84 (Aryl CH), 81.37 [C(CH₃)₃], 65.76 (PhCH₂), 60.29 (α-C_{Leu}), 49.47 (α-C_{Val}), 42.22 (NHCH₂), 31.03 (NCH₃), 27.92 [C(CH₃)₃], 27.06 (NHCH₂CH), 24.51 (NCH₃CHCH), 23.32, 21.79, 19.78 and 18.83 (4×CH₃); E.S.M.S. m/z 457 (M+Na, 32 ), 435 [M+1, 100%], 391 (6), 379 (29), 361 (6), 262 (4), 214 (3).

### Preparation of N-Carbonylbenzyloxy-L-Phe-L-N-Methyl-Phe-tert-Butyl Ester

R²=benzyl

R=benzyl

R″=benzyl

The N-methyl phenylalanine ester (350 mg, 0.8 mmol) was dissolved in dry dichloromethane (4 ml) and stirred under a nitrogen atmosphere at 0° C. The phenylalanine acid (1.2 eq, 295 mg, 1.0 mmol) was added along with PyBroP (1.2 eq, 460 mg, 1.0 mmol) and DIPEA (4.0 eq, 573 μl, 3.3 mmol). The reaction was stirred for 2 h at 0° C. The mixture was then passed through a celite pad and the cake was washed with methanol. The solution was concentrated in vacuo and the residue was subjected to flash column chromatography, elutin with 30% ethyl acetate-hexane to give the dipeptide as a lightly opaque residue (235 mg, 55%); [α]²⁵_{D}=−49.6 (c, 0.45 in CH₂Cl₂); vₘₐₓ (NaCl)/cm⁻¹ 3308 (NH), 2977 (Aryl CH), 2933 (saturated CH), 1727 and 1647 (2×C═O), 1497, 1249, 1155, 1048, 740, 698; ¹H NMR (300 MHz, CDCl₃) (rotamers) δ 7.34-7.09 (15 H, m, ArH), 5.41-4.60 (5 H, m, NH, PhCH₂ and 2×α-H), 3.32-2.30 (7 H, m, NHCHCH₂, NCH₃ and NCH₃CHCH₂), 1.41 [9 H, s, C(CH₃)₃]; ¹³C NMR (75 MHz, CDCl₃) (rotamers) δ 171.51, 169.34 and 155.40 (3×C═O), 136.95, 136.18, 136.00 (Aryl C), 129.45, 129.30, 129.10, 128.82, 128.38, 128.33, 128.14, 127.97, 127.80, 127.67, 127.07, 126.76, 126.53 (Aryl CH), 81.81 [C(CH₃)₃], 66.48 (PhCH₂), 62.14 (α-C_{MePhe}), 51.83 (α-C_{Phe}), 38.67 (NCH₃CHCH₂), 34.70 (NHCHCH₂), 32.73 (NCH₃), 27.88 [C(CH₃)₃]; E.S.M.S. m/z 539 (M+Na, 8%), 517 [M+1, 100%], 461 (22), 443 (6).

### Preparation of N-9-Fluorenylmethoxycarbonyl-L-Val-L-N -Methyl-Thr(O-benzyl)-tert-Butyl Ester

R²=CH(OBn)CH₃

R=isopropyl

R″=9-fluorenylmethyl

The N-Fmoc-N-methyl threonine ester (520 mg, 1.03 mmol) was dissolved in 33% diethylamine/DMF (6 ml) at room temperature for 1 h. The reaction mixture was concentrated in vacuo to a residue. The residue was dissolved in dry dichloromethane (6 ml) and stirred under a nitrogen atmosphere at 0° C. The valine acid (1.2 eq, 457 mg) was added along with PyBroP (627 mg, 1.3 eq) and DIPEA (3.0 eq, 0.541 ml). The reaction was left to stir at room temperature overnight. The mixture was diluted with ether (30 ml) and was then washed sequentially with dilute hydrochloric acid, saturated sodium bicarbonate solution, brine, dried (MgSO₄), filtered and evaporated to dryness at reduced pressure. The residue was subjected to flash column chromatography, eluting with 20% ethyl acetate-hexane to give the dipeptide as a clear colourless oil (510 mg, 82%). A small portion was chromatographed for a second time for analytical purposes; [α]²⁵_{D}=+49.6 (c, 1.0 in CHCl₃) ; vₘₐₓ (NaCl)/cm⁻¹ 3302 (NH), 3089 3066, 3038 (Aryl CH), 3000-2800 (saturated CH), 1727 and 1643 (2×C═O), 1525, 1506, 1499, 1479, 1451, 1369, 1299, 1160, 1110, 1088, 1030, 758, 738, 698; ¹H NMR (300 MHz, CDCl₃) δ 7.75-7.24 (13 H, m, ArH), 5.81, (1H, d, J=9.2 Hz, NH), 5.43 (1 H, d, J=4.2 Hz, β-CH-Thr), 4.72-4.20 (7 H, m, NCHCO+2, CHCH₂, PhCH₂), 3.30 (3 H, s, NCH₃), 1.44-1.39 [9 H, m, C(CH₃)₃]1, 1.17 (3 H, d, J=6.2 Hz, CH₃), 1.08 (3 H, d, J=6.6 Hz, CH₃), 1.01 (3 H, d, J=6.6 Hz, CH₃); ¹³C NMR (75 MHz, CDCl₃) (rotamers) δ 173.57, 168.16 (2×C═O), 156.18 (C═O), 143.70, 143.60, 141.01, 138.14 (Aryl C), 127.97, 127.39, 127.17, 126.95, 126.79, 124.89, 124.08, 119.67 (Aryl CH), 81.51 [C(CH₃)₃), 75.02 (CHO), 71.45 (CH2), 66.69 (PhCH₂), 60.90, 55.39 (2×Cα), 46.94 (CH), 33.92 (NCH₃), 30.99 (NHCHCH), 27.78, 27.56 (tBu), 19.24, 17.41, 15.67 (CH₃×3).

### Preparation of N-9-Fluorenylmethoxycarbonyl-L-Leu-L-N-Methyl-Gly-tert-Butyl Ester

R²=H
R=isobutyl
R″=9-fluorenylmethyl

Sarcosine tert-butyl ester (500 mg, 3.7 mmol), triethylamine (0.3 ml) and N-Fmoc-leucine (1.4 g, 4 mmol) and PyBrop (1.75 g, 3.7 mmol) were added to dichloromethane (16 ml) in a 50 mL round bottomed flask with a magnetic stirring. The reaction mixture was left to stir at room temperature for 2.5 hours under an atmosphere of nitrogen. The solution was washed with dilute citric acid solution, sodium bicarbonate solution, brine, dried (MgSO₄), filtered and concentrated under reduced pressure. The crude residue was purified by column chromatography on silica eluting with 30% ethyl acetate-hexane to produce the dipeptide as a white solid (440 mg, 40%). ¹H NMR (300 MHz, CDCl₃) δ7.67-7.19 (8H, m, ArH), 5.82-5.79 (1H, m, NH), 4.70-4.50 (3H, m, NCH₂ and α-CH), 4.07-3.58 (3H, m, CHCH₂), 3.06-2.91 (3H, m, NCH₃), 1.90-0.84 [18H, m, CH₂CH(CH₃)₂ and tBu]. ¹³C NMR (75 MHz, CDCl₃) δ173.77, 172.03, 156.09, 143.57, 143.37, 140.90, 127.33, 126.71, 124.85, 119.59, 66.72, 49.51, 48.96, 46.78, 41.29, 36.17, 24.20, 23.00, 21.27.

### Preparation of N-9-Fluorenylmethoxycarbonyl-L-Phe-L-N-Methyl-Gly-tert-Butyl Ester

R²=H
R=isobutyl
R″=9-fluorenylmethyl

Sarcosine tert-butyl ester (350 mg, 2.7 mmol), triethylamine (0.25 ml), N-Fmoc phenylalanine (1.0 g, 2.6 mmol) and PyBrop (1.1 g, 2.5 mmol) were added to dichloromethane (10 ml) in a 50 mL round bottomed flask with a magnetic stirring. The reaction mixture was left to stir at room temperature for 1 hour under an atmosphere of nitrogen. The reaction was monitored by tlc. The solution was washed with dilute citric acid solution, sodium bicarbonate solution, brine, dried (MgSO₄), filtered and concentrated at reduced pressure. The crude residue was purified by column chromatography on silica eluting with 30% ethyl acetate-hexane to produce the dipeptide as a white solid (600 mg, 61%). ¹H NMR (300 MHz, CDCl₃) δ7.76-7.20 (13H, m, ArH), 5.81-5.78 (1H, m, NH), 5.02-5.00 (1H, m, α-CH Phe), 4.37-4.03 (7H, m, CHCH₂, NCH₂, 2×αCH), 3.11-2.93 (5H, m, NCH₃, CHCH₂), 1.53-1.45 (9H, m, tBu). ¹³C NMR (75 MHz, CDCl₃) δ171.49, 167.41, 155.3, 143.58, 140.93, 136.01, 135.73, 129.28, 129.12, 128.06, 127.32, 126.71, 126.61, 124.88, 119.59, 81.63, 66.68, 50.06, 38.85, 35.89, 27.73, 27.65.

### Preparation of N-9-Fluorenylmethoxycarbonyl-L-Pro-L-N-Methyl Ala-tert-Butyl Ester

R²=methyl
R=CH₂CH₂CH₂ (proline ring)
R″=9-fluorenylmethyl

N-Methyl alanine tert-butyl ester (360 mg, 2.4 mmol), triethylamine (0.42 ml), N-Fmoc proline (810 mg, 2.4 mmol) and PyBrop (1.2 g, 1.2 eq.) were added to dichloromethane (10 ml) in a 50 mL round bottomed flask with magnetic stirring. The reaction mixture was left to stir at room temperature for 12 h under an atmosphere of nitrogen. The reaction was monitored by tic. The reaction mixture was washed with dilute citric acid solution, sodium bicarbonate solution, brine, dried (MgSO₄), filtered and concentrated at reduced pressure. The crude residue was purified by column chromatography on silica with 30% ethyl acetate-hexane to produce the didpeptide as a white solid (50 mg, 4.4%). ¹H NMR (300 MHz, CDCl₃) δ7.76-7.28 (8H, m, ArH), 4.96-4.25 (7H, m, 2×αH, NCH₂, CHCH₂), 3.11 and 2.93 (3H, 2s, NCH₃), 1.61-0.48 (16H, m, tBu, CHCH₃, CH₂CH₂).

### Example 10

### Preparation of 3-Benzyloxycarbonyl-4,4-dimethyl-oxazolidin-5-one (Z-AIB Oxazolidinone)

Z-AIB—OH (1.3 g, 5.5 mmol) was suspended in toluene (50 mL) in a 100 mL round bottomed flask fitted with a condenser. Paraformaldehyde (1.0 g) and a catalytic amount of camphorsulfonic acid were added, and the mixture was refluxed for 1.5 hours. The cooled solution was washed with 10% sodium bicarbonate, and the organic phase was concentrated at reduced pressure. The residue was purified by chromatography on silica gel, eluting with 30% ethyl acetate-hexane, producing the oxazolidinone as a clear oil (1.01 g, 80%). ¹H NMR (300 MHz, CDCl₃) 7.39-7.34 (m, 5H), 5.27 (s, 2H), 5.16 (s, 2H), 1.57 (s, 6H). ¹³C NMR (75 MHz, CDCl₃) 175.47, 135.51, 128.62, 128.44, 128.12, 76.127, 67.29, 56.97, 22.54. Anal. Calcd for C₁₂H₁₅O₄N C, 62.64; H, 6.07; N, 5.62. Found C, 62.74; H, 6.07, N, 5.67%.

### Example 11

### Preparation of N-Benzyloxycarbonyl-N-methyl α-Amino Isobutyric acid

Z-AIB—OH (1.17 g, 4.7 mmol) was dissolved in chloroform* (11.2 mL) and trifluoroacetic acid (11.2 mL) and the mixture was heated to 50° C. with stirring. To this mixture was added triethylsilane (1.65 mL, 15 mmol) and stirred at 50° C. for 16 hours. The solution was concentrated at reduced pressure, diluted with chloroform* (25 mL) and then concentrated again at reduced pressure. Purification of the residue was achieved by chromatography on silica, eluting with 5% methanol-chloroform, producing a white solid (800 mg, 68%). *Chloroform was washed with water and dried over magnesium sulphate to remove the ethanol stabiliser. ¹H NMR (300 MHz, CDCl₃), (s, 1H), (m, 5H), (s, 2H), (s, 3H), (s, 6H). ¹³C NMR (75 MHz, CDCl₃) 197.94, 197.54, 156.13, 136.10, 128.36, 127.93, 127.87, 67.55, 60.74, 29.63, 23.62.

### Example 12

### Preparation of (S)-3-Benzyloxycarbonyl-4-(3-hydroxypropyl)-oxazolidin-5-one

Z-glutamic acid (500 mg) was dissolved in dry THF (5 mL), under a nitrogen atmosphere with stirring. To the solution was added 1M THF.BH₃ complex (3.4 mL), and the mixture was left to stir overnight. The solution was concentrated in vacuo and the residue was left to stand (the boric acid precipitates), dissolved in dichloromethane, filtered and further purified by chromatography on silica, eluting with 40% ethyl acetate-hexane to produce a clear oil (150 mg, 31%). ¹H NMR (300 MHz, CDCl₃) 7.58-7.37 (m, 5H), 5.50 (br s, 1H), 5.20-5.07 (m, 2H), 2.46 (br s, 2H), 2.33-2.12 (m, 2H). ¹³C NMR (75 MHz, CDCl₃) 171.74, 152.26, 135.00, 128.24, 128.06, 127.95, 77.48, 67.56, 63.67, 32.92, 18.44.

### Example 13

### Preparation of (S)-3-Benzyloxycarbonyl-4-vinyl-oxazolidin-5-one

Z-Allylglycine-OH (500 mg) in toluene (20 mL), in a 100 mL round bottomed flask, was fitted with a water condenser. To this mixture was added paraformaldehyde (0.2 g) and a catalytic amount of camphorsulfonic acid and the mixture was refluxed for 2 hours. The solution was concentrated in vacuo, and purified on silica gel, eluting with 30% ethyl acetate-hexane to produce a clear oil (410 mg). ¹H NMR (300 MHz, CDCl₃) 7.40-7.36 (m, 5H), 5.24-5.09 (m, 3H), 4.43 (br s, 1H), 2.60 (br s, 2H). ¹³C (75 MHz, CDCl₃) 173.36, 152.15, 135.15, 128.25, 128.06, 127.50, 120.62, 77.79, 67.49, 54.63.

### REFERENCES

1. Fischer, E., and Lipschitz, W., Ber. Dtsch. Chem. Ges., 1915, 48, 360.
2. Coggins, J. R., and Benoiton, N. L., Can., J. Chem., 1971, 49, 1968; McDermott, J. R., and Benoiton, L. N., Can. J. Chem., 1973, 51, 1915, 2555, 2562; Benoiton, L. N., Kuroda, K., Cheung, S. T., and Chen, F. M. F., Can. J. Biochem., 1979, 57, 776.
3. Hlavácek, J., Poduska, K., Sorm. F., and Slama, K., Collect. Czech. Chemm. Commun., 1976, 41, 2079; Hlavácek, J., Fric, I., Budesinsky, M., and Blaha, K., Colelct. Czech. Chem. Commun., 1988, 53, 2473.
4. Olsen, R. K., J. Org. Chem., 1970, 35, 1912.
5. Okamoto, K., Abe, H., Kuromizu, K., and Izumiya, N., Mem. Fac. Sci. Kyushu Univ. Ser. C, 1974, 9, 131.
6. Ohfune, Y., Kurokawa, N., Higuchi, N., Saito, M., Hashimoto, M., and Tanaka, T., Chem. Lett., 1984, 441.
7. Ramanjulu, J. M. and Joullié, M. M., Synth. Commun., 1996, 26, 1379.
8. Chruma, J. J., Sames, D., and Polt, R., Tetrahedron Lett., 1997, 38, 5085.
9. Quitt, P., Hellerbach, J., and Vogler, K., Helv, Chim. Acta, 1963, 46, 327; Ebata, M., Takahashi, Y., and Otsuka, H., Bull. Chem. Soc. Jpn, 1966, 39, 2535.
10. Brockmann, H., and Lackner, H., Chem. Ber, 1967, 100, 353.
11. Peter, H., Brugger, M., Schreiber, J., and Eschenmoser, A., Helv. Chim. Acta, 1963, 46, 577.
12. O'Donnell, M. J., Bruder, W. A., Daugherty, B. W., Liu, D., and Wojciechowski, K., Tetrahedron Lett., 1984, 25, 3651; O'Donnell, M. J., and Polt, R. L., J. Org. Chem., 1982, 47, 2663.
13. Auerbach, J., Zamore, M., and Weinreb, S. M., J. Org. Chem., 1976, 41, 725.
14. Dorow, R. L., and Gingrich, d. E., J. Org. Chem., 1995, 60, 4986.
15. Wisniewski, K., and Kolodziejczyk, A. S., Tetrahedron Lett., 1997, 38, 483.
16. Coulton, S., Moore, G. A., and Ramage, R., Tetrahedron Lett., 1976, 4005.
17. Luke, R. W. A., Boyce, P. G. T., and Dorling, E. K., Tetrahedron Lett., 1996, 37, 263.
18. Spengler, J., and Burger, K., Synthesis, 1998, 67.
19. Freidinger, R. M., Hinkle, J. S., Perlow, D. S., and Arison, B. H., J. Org. Chem., 1983, 48, 77.
20. D. Ben-Ishai, J. Am. Chem. Soc., 1957, 79, 5736.
21. Itoh, M., Chem. Pharm. Bull., 1969, 17, 1679.
22. Reddy, G. V., Rao, G. V., and Iyengar, D. S., Tetrahedron Lett., 1998, 39, 1985.
23. Williams, R. M., and Yuan, C., J. Org. Chem., 1994, 59, 6190.
24. Grieco, P. A., and Bahsas, A., J. Org. Chem., 1987, 52, 5746.
25. Effenberger, F., Burkard, U., and Willfahrt, J., Liebigs Ann. Chem., 1986, 314.
26. Oppolzer, W., Cintas-Moreno, P., Tamura, O., and Carbinaux, F., Helv, Chim. Acta, 1993, 76, 187.
27. Aurelio, L., Brownlee, R. T. C., Hughes, A. B., and Sleebs, B. E., Aust. J. Chem., 2000, 53, 425.
28. Freidinger, R. M., Hinkle, J. S., Perlow, D. S., and Arison, B. H., J. Org. Chem., 1983, 48, 77.
29. Perrin, D. D., and Armarego, W. L. F., Purification of Laboratory Chemicals, 3^{rd} Edn (Pergamon: Oxford 1988).
30. Reddy, G. V., Rao, G. V., and Iyengar, D. S., Tetrahedron Lett., 1998, 39, 1985.
31. Mizoguchi, T., Levin, G., Woolley, D. W., and Stewart, J. M., J. Org. Chem., 1968, 33, 903.
32. Chen, S.-T., Wu, S.-H., and Wang, K.-T., Synth. Commun., 1989, 19, 3589.
33. Wang, J., Okada, Y., Li, W., Yokio, T., and Zhu, J., J. Chem. Soc., Perkin Trans 1, 1997, 621.
34. Wilchek, M., and Patchornik, A., J. Org. Chem., 1964, 29, 1629.
35. Ondetti, M. A., J. Med. Chem., 1963, 6, 10.
36. Dawson, J. R., Darg, Y. L., Mellor, J. M., and McAleer, J. F., Tetrahedron, 1996, 52, 1361; Davies, J. S., Hassall, C. H., and Hopkins, K. H., J. Chem. Soc., Perkin Trans 1, 1973, 2614.
37. Zervas, L., Photaki, I. and Ghelis, N., J. Am. Chem. Soc., 1963, 85, 1337.
38. Clark, D. G. and Cordes, E. H., J. Org. Chem., 1973, 38, 270.
39. Yamashiro, D., Aanning, H. L., Branda, L. A., Cash, W. D., Murti, V. V. S., and De Vigneaud, V., J. Am. Chem. Soc., 1968, 90, 4141.
40. Greene, T. W. and Wuts, P. G. M., In “Protective Groups in Organic Synthesis”, 2^{and} Edition, Wiley-Interscience, New York, 1991.
41. Tam, J. P., Heath, W. F., and Merrifield, R. B., J. Am. Chem. Soc., 1983, 105, 6442; Ogawa, H., Sasaki, T., Irie, H., and Yajima, H., Chem. Pharm. Bull., 1978, 26, 3144; Yajima, H., Takeyama, M., Kanaki, J., Nishimura, O., and Fujino, M., Chem. Pharm. Bull., 1978, 26, 3752; Yajima, H., Futaki, S., Otaka, A., Yamashita, T., Funakoshi, S., Bessho, K., Fujii, N., and Kenichi, A., Chem. Pharm. Bull., 1986, 34, 4356.
42. Mzengeza, S., and Whitney, R. A., J. Org. Chem., 1988, 53, 4074.
43. Fujii, N., Sasaki, T., Funakoshi, S., Irie, H., and Yajima, H., Chem. Pharm. Bull., 1978, 26, 650; Holland, H. L., Andreana, P. R., and Brown, F. M., Tetrahedron Asymm., 1999, 10, 2833; Strazzolini, P., Scuccato, M., and Giumanini, A. G., Tetrahedron, 2000, 56, 3625.
44. Nicolás, E., Vilaseca, M., and Giralt, E., Tetrahedron, 1995, 51, 5701.
45. Sieber, P. and Riniker, B., Tetrahedron Lett., 1991, 32, 739.
46. Kim, K., Lin, Y.-T., and Mosher, H. S., Tetrahedron Lett., 1988, 29, 3183.
47. Feichtinger, K., Sings, H. L., Baker, T. J., Matthews, K., and Goodman, M., J. Org. Chem., 1998, 63, 8432.
48. De Boer, T. J., and Backer, H. J., Org. Synth. Coll. Vol., 1963, 4, 250.
49. Fukuyama, T., Liu, G., Linton, S. D., Lin, S. C., and Nishino, H., Tetrahedron Lett., 1993, 34, 2577; Fukuyama, T., Lin, S. C., and Li. L., J. Am. Chem. Soc., 1990, 112, 7050.
50. Maienfisch, P., Huerlimann, H., and Haettenschwiler, J., Tetrahedron Lett., 2000, 41, 7187.
51. Previero, A., Coletti-Previero, M. A., and Cavadore, J.-C., Biochim. Biophys. Acta, 1967, 147, 453.
52. Daly, J. W., Mauger, A. B., Yonemitsu, O., Antonov, V. K., Takase, K., and Witkop, B., Biochemistry, 1967, 6, 648.
53. Joucla, M., and Mortier, J., Bull. Soc. Chim. France, 1988, 579.
54. Lin, N.-H., He, Y., Elliott, R. L., Chorghade, M. S., Wittenberger, S. J., Bunnelle, W. H., Narayanan, B. A., Singam, P. R., Esch, K. J., Beer, D. O., Witzig, C. C., Herzig, T. C. and Rao, A. V. R., PCT Int. Appl. 1995, WO9507277, Chem. Abs., 123, 9432.
55. See references 1-27 in Ref 27.
56. Yamashiro, D., Aanning, H. L., Branda, L. A., Cash, W. D., Murti, V. V. S., and Du Vigneaud, V., J. Amer. Chem. Soc., 1968, 90, 4141; see also Ratner, S., and Clarke, H. T., J. Am. Chem. Soc., 1937, 59, 200.
57. Reddy, G. V., and Iyengar, D. S., Chem. Lett., 1999, 299.
58. Sokolov, V. V., Kozhushkov, S. I., Nikolskaya, S., Belov, V. N., Es-Sayed, M., and De Meijere, A., Eur. J. Org. Chem., 1998, 777.
59. Hutton, G. E., PCT Int. Appl. 1996, WO9611181, Chem. Abs., 125, 115135; Adger, B., Dyer, U., Hutton, G., and Woods, M., Tetrahedron Lett., 1996, 37, 6399.
60. Walter, M. W., Adlington, R. M., Baldwin, J. E., and Schofield, C. J., J. Org. Chem., 1998, 63, 5179.
61. Siepmann, E., and Zahn, H., Biochim. Biophys. Acta, 1964, 82, 412.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A compound of formula I or II: 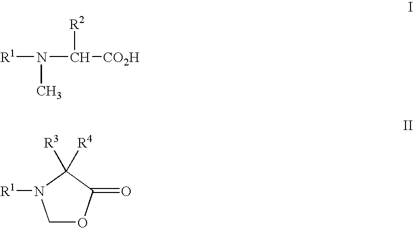
in which
R¹ is an N-protecting group or a peptide;
R² is CHCH₃OAc or CHR⁵R⁶ in which R⁵ is hydrogen and R⁶ is OAc, CONH₂, SBn, 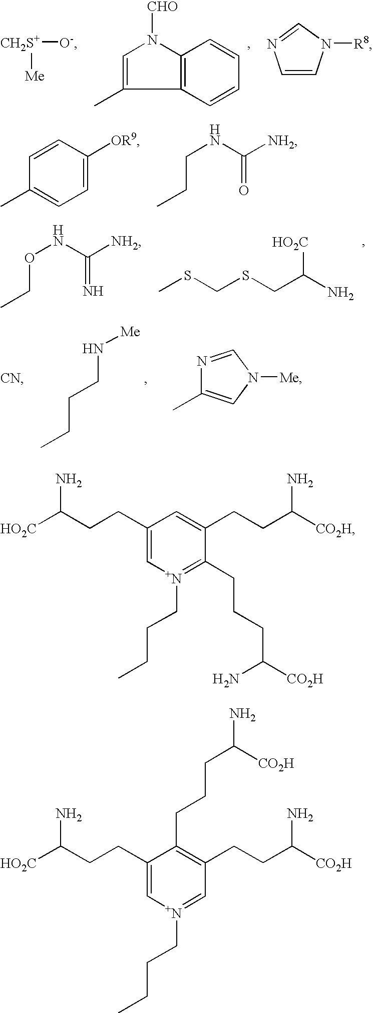
CO2R7 or CH2CO2R7 in which R7 is a carboxyl protecting group; and
R3 is CHCH3OAc, 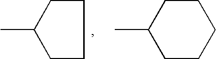
or CHR5R6 in which R5 is as defined above and R6 is OAc, SBn, CONHTrt, 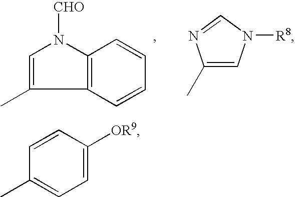
CO₂R⁷, CHCO₂R⁷, CH₂CH₃ or CH═CH₂ in which R⁷ is as defined above, R⁸ is a histidine protecting group and R⁹ is a phenol protecting group;
R⁴ is hydrogen or R⁴ is methyl when R³ is OAc;
R³ together with R⁴ forms cyclopentyl; or
R² and R³ independently represent optionally protected amino acid side chains selected from: 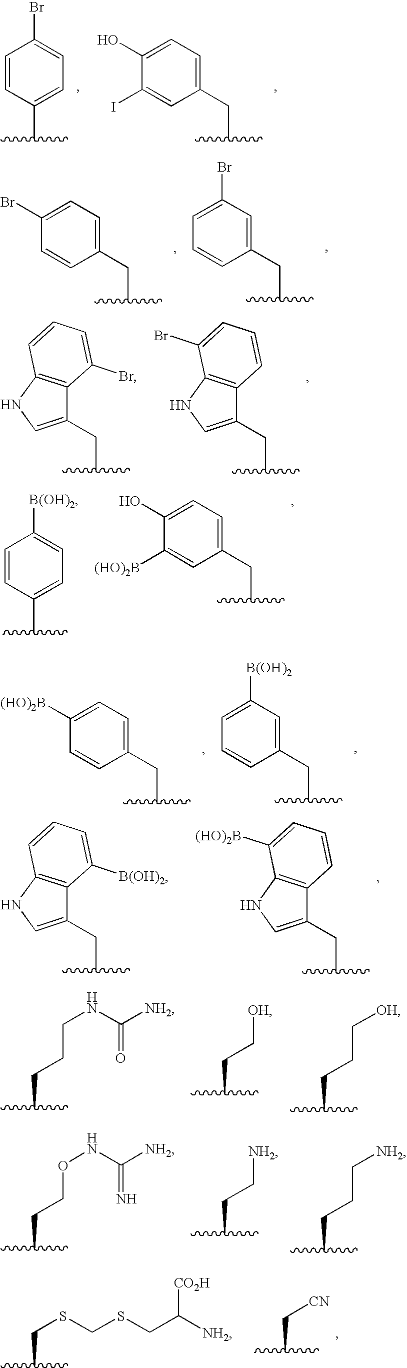 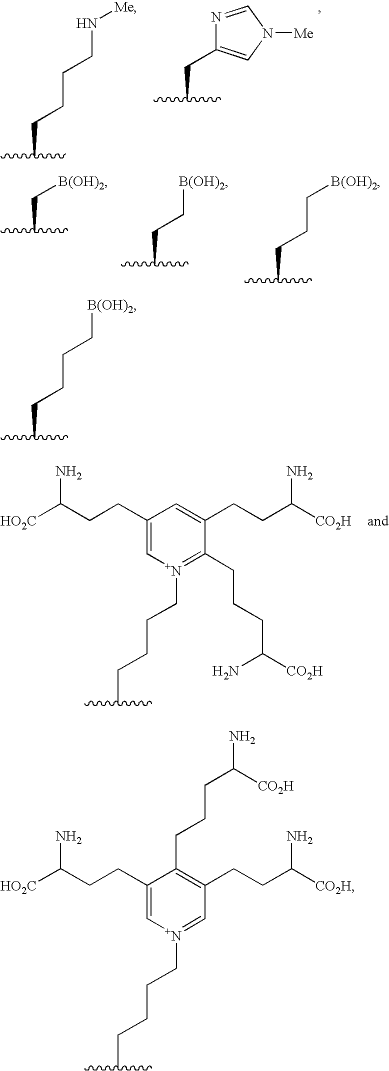
salts, hydrates, solvates, derivatives, tautomers and/or isomers thereof.

2. A compound according to claim 1, which is selected from: 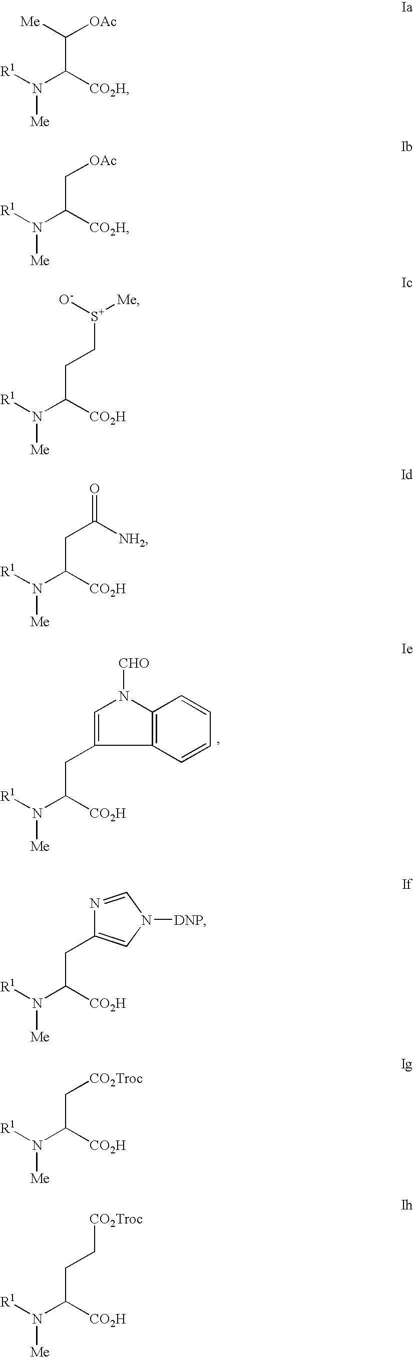 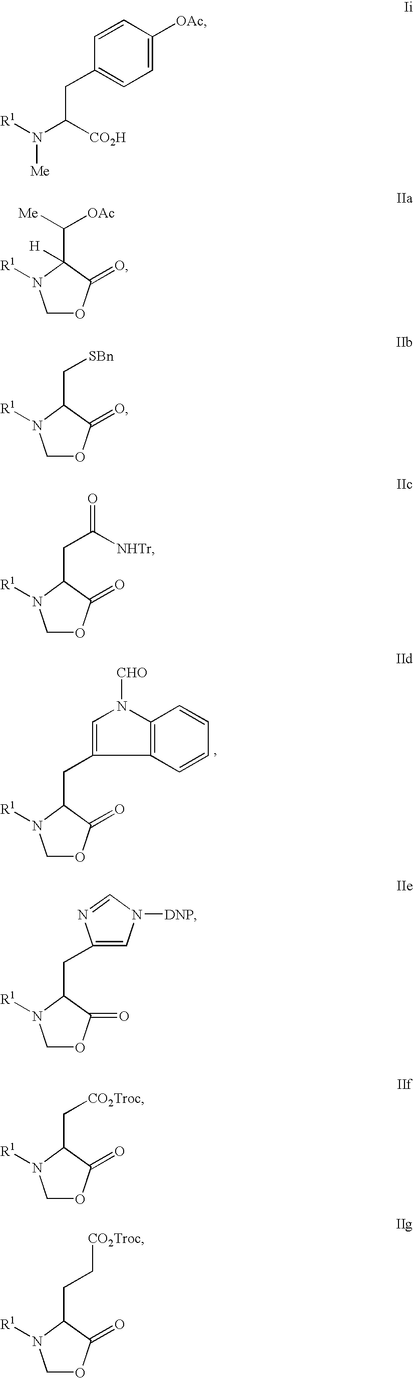 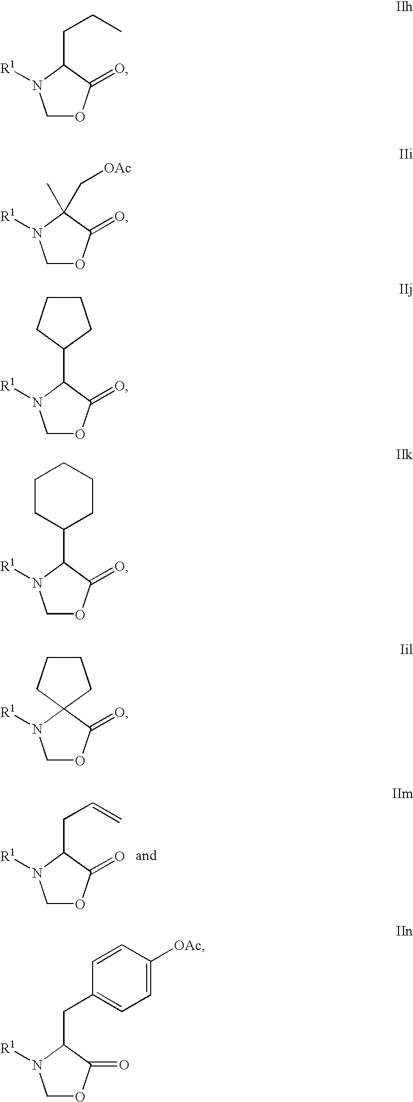
in which R¹ is as defined in claim 1.

3. A process for preparing the compound of formula I as defined in claim 1 which comprises reductive cleavage of the compound of formula II as defined in claim 1.

4. A process according to claim 3 in which the reductive cleavage employs trifluoroacetic acid (TFA) as the acid and triethylsilane (Et₃SiH) as the reductant.

5. A process for preparing the compound of formula I or II as defined in claim 1 when
R¹ is an N-protecting group or a peptide;
R² is CHCH₃OAc or CHR⁵R⁶ in which R⁵ is hydrogen and R⁶ is OAc, CONH₂, SBn, 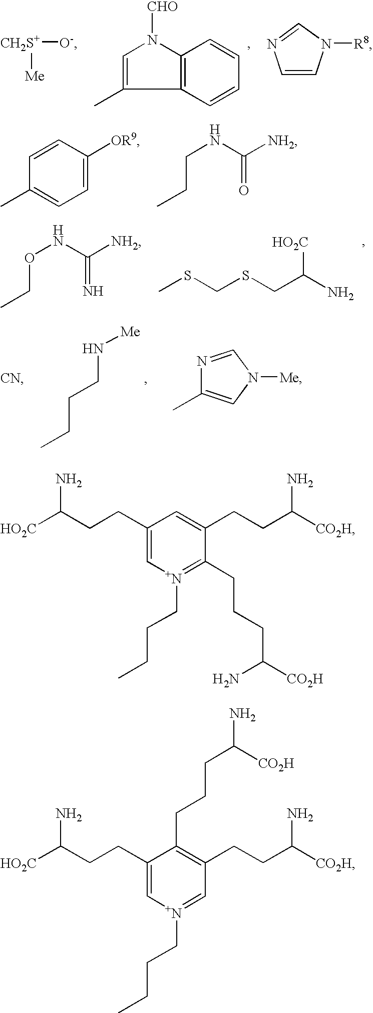
CO₂R⁷ or CH₂CO₂R⁷ in which R⁷ is a carboxyl protecting group; and
R³ is CHCH₃OAc, 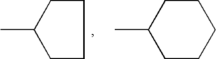
or CHR⁵R⁶ in which R⁵ is as defined above and R⁶ is OAc, SBn, CONHTrt, 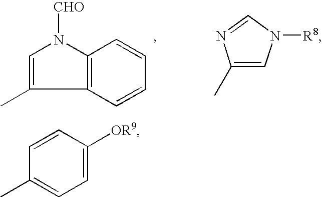
CO₂R⁷ CHCO₂R⁷, CH₂CH₃ or CH═CH₂ in which R⁷ is as defined above, R⁸ is a histidine protecting group and R⁹ is a phenol protecting group;
R⁴ is hydrogen or R⁴ is methyl when R³ is OAc;
R³ together with R⁴ forms cyclopentyl; which comprises the steps of:
(a) converting a compound of formula III 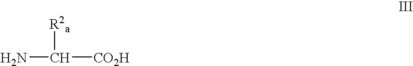
in which
R²ₐ is CHOHMe or CHR⁵R⁶ₐ in which R is as defined above and R⁶ₐ is OH, SH, CONH₂, 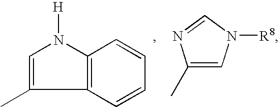
in which R⁸ is as defined above, 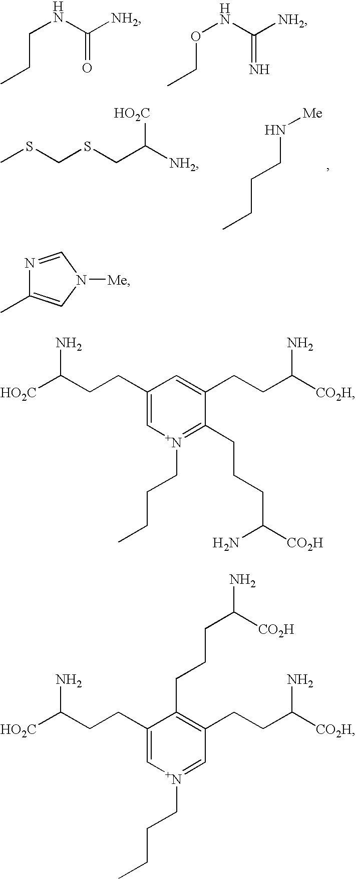
CO₂H or CH₂CONH₂ or salts thereof into a compound of formula IV 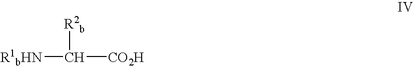
in which
R¹_{b} is an N-protecting group;
R²_{b} is CHOAcMe or CHR⁵R⁶_{b} in which R⁵ is as defined above and R⁶_{b} is OAc, SBn, SMe, CONHR¹_{b} in which R¹_{b} is as defined above, 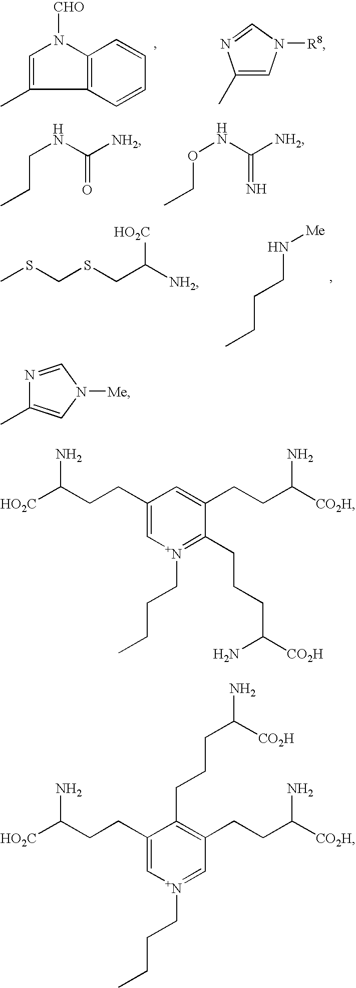
CO²H or CH₂CO₂H;
(b) oxazolidination of the compound of formula IV to form the compound of formula II; and
(c) reductive cleavage of the compound of formula II to form the compound of formula I.

6. A process according to claim 5, in which the conversion step (a) results in the protection of the amino group on the compound of formula III to produce the compound of formula IV.

7. A process according to claim 5, in which the oxazolidination step (b) uses a formaldehyde source in an organic solvent.

8. A process according to claim 7, in which the formaldehyde source is paraformaldehyde and paratoluenesulphonic acid (TsOH).

9. A process according to claim 7, in which the organic solvent is benzene or toluene.

10. Use of the compound of formula I or II defined in claim 1 in the synthesis of peptides.

11. A peptide which includes the compound of formula I or II as defined in claim 1.

12. A peptide according to claim 11, which is a dipeptide.

13. A peptide according to claim 12, in which the dipeptide is of the formula V 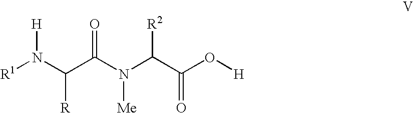
in which
R¹ and R² are as defined in claim 1 or claim 2, R′ is an optionally protected amino acid side chain and R is H or a carboxyl-protecting group.

14. A kit for use in synthesising peptides which comprises
(a) at least one compound of formula I or formula II as defined in claim 1; and
(b) optionally at least one other N-methyl amino acid, its precursor oxazolidinones, an optionally substituted amino acid, or protected forms thereof,
said compounds, N-methyl amino acids, oxazolidinones and/or amino acids being held separately.

15. A kit for use in synthesising peptides which comprises
(a) peptide as defined in claim 11; and
(b) optionally at least one other N-methyl amino acid, its precursor oxazolidinones, an optionally substituted amino acid, or protected forms thereof,
said compounds, N-methyl amino acids, oxazolidinones and/or amino acids being held separately.
